(19) United States
(12) Patent Application Publication
(10) Pub. No.: US 2015/0030593 A1
(43) Pub. Date: Jan. 29, 2015

(54) COMPOSITIONS OF PENETRATION-ENHANCED TARGETING PROTEINS AND METHODS OF USE

(71) Applicant: PERMEON BIOLOGICS, INC., CAMBRIDGE, MA (US)

(72) Inventors: Katherine S. Bowdish, Boston, MA (US); James S. Huston, Newton Lower Falls, MA (US); Erik M. Vogan, Medford, MA (US)

(73) Assignee: PERMEON BIOLOGICS, INC., CAMBRIDGE, MA (US)

(21) Appl. No.: 14/214,463

(22) Filed: Mar. 14, 2014

Related U.S. Application Data
(60) Provisional application No. 61/800,295, filed on Mar. 15, 2013.
(60) Provisional application No. 61/800,162, filed on Mar. 15, 2013.

(51) Int. Cl. C07K 16/32; C07K 14/435; A61K 31/5365; A61K 39/395; A61K 38/14

(52) U.S. Cl. CPC C07K 16/32; A61K 39/3955; A61K 38/14; A61K 31/5365; C07K 14/43595; C07K 2317/54; A61K 2039/505
USPC 424/134.1; 530/387.3; 536/23.4; 435/320.1; 435/252.33; 435/69.7; 435/375; 435/254.2; 435/328

(57) ABSTRACT

The disclosure relates to penetration-enhanced targeted proteins and their uses for therapeutics delivery.

### RELATED APPLICATIONS

This application claims the benefit of priority from U.S. provisional application Ser. No. 61/800,295, filed Mar. 15, 2013 and 61/800,162, filed Mar. 15, 2013. The disclosures of each of the foregoing applications are hereby incorporated by reference in their entirety.

### BACKGROUND OF THE DISCLOSURE

The effectiveness of an agent intended for use as a therapeutic, diagnostic, or in other applications is often highly dependent on its ability to reach a cell or tissue type of interest and further penetrate the cellular membranes or tissues of those cell or tissue types of interest to induce a desired change in biological activity. Although many therapeutic drugs, diagnostic or other product candidates, whether protein, nucleic acid, small organic molecule, or small inorganic molecule, show promising biological activity in vitro, many fail to reach or penetrate the appropriate target cells to achieve the desired effect, often due to physiochemical properties that result in inadequate targeted biodistribution in vivo.

### SUMMARY OF THE DISCLOSURE

The disclosure provides penetration-enhanced targeted proteins (PETPs). PETPs are protein entities that comprise at least two regions (the PETP core): a target binding region that binds a cell surface target and a charged protein moiety (CPM) that promotes internalization in to cells. By combining the features of these two regions, the disclosure provides a protein entity with cell targeting ability and also cell penetration capability (e.g., the protein entity penetrates cells). This provides a platform for preferentially enhancing penetration of molecules into cells. Ancillary agents, including proteins, peptides, nucleic acid molecules, and small molecules (e.g., therapeutic or cytotoxic drugs) can be connected, directly or indirectly, to this PETP core to enhance penetration of those ancillary agents, thereby delivering them across cellular membranes and into cells. Moreover, ancillary agents, such as small molecule drugs, may be co-administered with a PETP protein entity and, though not physically linked, the PETP protein entity can increase penetration and/or availability of the ancillary agent in the cytoplasm or nucleus of the cell. These features of PETP protein entities make them suitable for a range of in vitro and in vivo applications.

The disclosure provides penetration-enhanced targeted proteins (PETPs). PETPs are protein entities that comprise at least two regions (the PETP core): a target binding region that binds a cell surface target at the cell surface and a charged protein moiety (CPM) that promotes internalization in to cells. By combining the features of these two regions, the disclosure provides a protein entity with cell targeting ability and also cell penetration capability (e.g., the protein entity penetrates cells). This provides a platform for enhancing penetration of molecules into cells preferentially. In this way, both the target binding region and the CPM effect penetration. Ancillary agents, including proteins, peptides, nucleic acid molecules, and small molecules (e.g., therapeutic or cytotoxic drugs) can be connected, directly or indirectly, to this PETP core to enhance penetration of those ancillary agents, thereby delivering them across cellular membranes and into cells. Moreover, ancillary agents, such as small molecule drugs, may be co-administered with a PETP protein entity and, though not physically linked, the PETP protein entity can increase penetration and/or availability of the ancillary agent in the cytoplasm or nucleus of the cell. These features of PETP protein entities make them suitable for a range of in vitro and in vivo applications.

In one aspect, the present disclosure provides a protein entity comprising: a target binding region that binds a cell surface target with a dissociation constant (K_{D}) of greater than 0.01 nM or with an avidity of greater than 0.001 nM, and a charged protein moiety (CPM) that enhances penetration into cells; wherein the CPM has tertiary structure and a molecular weight of at least 4 kDa, wherein the CPM has surface positive charge and a net theoretical charge of less than +20; wherein the cell surface target is distinct from that bound by the CPM; and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone. In certain embodiments, effective penetration refers to the preferential enhancement of cell penetration of the protein entity as a function of expression of the cell surface target.

In a related aspect, the present disclosure provides a protein entity comprising: a target binding region that binds a cell surface target with a dissociation constant (K_{D}) of less than 1 μM or with an avidity of less than 1 μM, and a charged protein moiety (CPM) that enhances penetration into cells; wherein the CPM has tertiary structure and a molecular weight of at least 4 kDa, wherein the CPM has surface positive charge and a net theoretical charge of less than +20; wherein the cell surface target is distinct from that bound by the CPM; and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone. In certain embodiments, effective penetration refers to the preferential enhancement of cell penetration of the protein entity as a function of expression of the cell surface target.

An additional aspect of the disclosure provides a protein entity comprising: a target binding region that binds a cell surface target with a dissociation constant (K_{D}) of greater than 0.01 nM or with an avidity of greater than 0.001 nM, and a charged protein moiety (CPM) that enhances penetration into cells; wherein the CPM has tertiary structure and a molecular weight of at least 4 kDa, wherein the CPM has surface positive charge, a net positive charge of at least +5, and a charge per molecular weight ration of less than 0.75; wherein the cell surface target is distinct from that bound by the CPM; and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone. In certain embodiments, effective penetration refers to the preferential enhancement of cell penetration of the protein entity as a function of expression of the cell surface target.

A further aspect of the present disclosure provides a protein entity comprising: a target binding region that binds a cell surface target with a dissociation constant (K_{D}) of less than 1 μM or with an avidity of less than 1 μM, and a charged protein moiety (CPM) that enhances penetration into cells; wherein the CPM has tertiary structure and a molecular weight of at least 4 kDa, wherein the CPM has surface positive charge, a net positive charge of at least +5, and a charge per molecular weight ration of less than 0.75; wherein the cell surface target is distinct from that bound by the CPM; and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone. In certain embodiments, effective penetration refers to the preferential enhancement of cell penetration of the protein entity as a function of expression of the cell surface target.

In certain embodiments of any of the foregoing aspects, a primary spacer region (SR) interconnects the target binding region and the CPM. In some embodiments, a primary spacer region (SR) forms a fusion protein with at least one unit of the target binding region and at least one unit of the CPM. The protein entity may further comprise an additional protein component connected to the CPM, the primary SR, or the target binding region. Optionally, the protein entity further comprises a cargo region connected to at least one of the CPM, the primary SR, or the target binding region. In some embodiments, the cargo region is selected from a peptide, a protein, or a small molecule. The protein entity may further comprise an additional spacer region (SR) interposed between the CPM and the adjacent additional protein component or cargo region, and optionally followed by additional SR-protein component units, each additional SR having the same or a distinct sequence from the primary SR.

In certain embodiments, the primary SR comprises all or a portion of an immunoglobulin (Ig) comprising at least one of a C_{H}1 domain, a hinge region, a C_{H}2 domain, and a C_{H}3 domain. Further, the primary SR may comprise an immunoglobulin (Ig) C_{H}1 domain that is genetically fused to a hinge region. Optionally, the primary SR further comprises a C_{H}2 domain of an immunoglobulin to interconnect a target binding region to a C-terminal C_{H}3 dimerization domain of an immunoglobulin. In certain embodiments, the SR does not comprises all or a portion of an Ig heavy chain. In certain embodiments, the SR comprises only one domain of an Ig, alone or as a pair of domains. In certain embodiments, the SR does not comprise a C_{H}2 domain.

In some embodiments, the CPM comprises a C_{H}3 domain of an immunoglobulin (Ig). The C_{H}3 domain may be a charge-engineered variant comprising least 3, at least 4, at least 5, at least 6, at least 7, or at least 8 amino acid substitutions to increase surface positive charge, theoretical net charge, and/or charge per molecular weight ratio. In certain embodiments, the CPM does not comprises a C_{H}3 domain

In some embodiments, the CPM comprises a C_{H}1 domain of an immunoglobulin. The C_{H}1 domain may be a charge-engineered variant comprising least 3, at least 4, at least 5, at least 6, at least 7, or at least 8 amino acid substitutions to increase surface positive charge, theoretical net charge, and/or charge per molecular weight ratio.

In some embodiments, the CPM comprises a C_{H}2 domain of an immunoglobulin. The C_{H}2 domain may be a charge-engineered variant comprising at least 3, at least 4, at least 5, at least 6, at least 7, or at least 8 amino acid substitutions to increase surface positive charge, theoretical net charge, and/or charge per molecular weight ratio.

In certain embodiments, the Ig is an IgG selected from the group consisting of IgG1, IgG2, IgG3, and IgG4. Optionally, the IgG is a human IgG.

In some embodiments, the target binding region is a target-specific Fv region, comprising a light chain variable (V_{L}) domain mated with a heavy chain variable (V_{H}) domain, together forming an antibody binding site that binds the cell surface target with suitable specificity and affinity. Optionally, the target binding region is a target-specific single chain Fv (scFv), comprising a light chain variable (V_{L}) domain fused via a linker of at least 12 residues with a heavy chain variable (V_{H}) domain, together forming an antibody binding site with suitable specificity and affinity. The V_{L} and V_{H} domain sequences may be human.

In some embodiments, the CPM comprises a portion of an immunoglobulin comprising two heavy chains, and wherein a distinct SR is used to connect each heavy chain to an additional protein module. Optionally, one or both of the V_{H} and V_{L} domains are human, humanized, murine, or CDR grafted, and wherein at least one of the V_{H} or V_{L} domains are optionally deimmunized.

In some embodiments, the protein entity comprises an immunoglobulin (Ig) C_{H}3 domain which has been altered to increase its surface positive charge and/or net positive charge to enhance penetration into cells. Further, the protein entity may comprise a pair of human C_{H}3 domains, of which the amino acid sequence of at least one domain has been altered to increase surface positive charge and/or net positive charge to enhance penetration into cells. Optionally, the amino acid sequences of both C_{H}3 domains are independently altered to increase surface positive charge and/or net positive charge to enhance penetration into cells.

In certain embodiments, the C_{H}3 domains are from human IgG and their charge engineering does not interfere with normal neonatal Fc receptor binding and cellular recycling. The C_{H}3 domains may be from human IgG and their charge-engineering modulates normal neonatal Fc receptor binding and cellular recycling in a manner that improves therapeutic efficacy of the protein entity.

In some embodiments, the CPM comprises an immunoglobulin (Ig) C_{H}3 domain which has been altered to increase its surface positive charge and/or net positive charge to enhance penetration into cells. Optionally, the CPM comprises a pair of human C_{H}3 domains, of which the amino acid sequence of at least one domain has been altered to increase surface positive charge and/or net positive charge to enhance penetration into cells. Further, the amino acid sequences of both C_{H}3 domains may be independently altered to increase surface positive charge and/or net positive charge to enhance penetration into cells. Altering of the amino acid sequence can comprise introducing at least 3, at least 4, at least 5, at least 6, at least 7, or at least 8 amino acid substitutions, independently, into one or, if present, both C_{H}3 domains to increase surface positive charge, net positive charge, and/or charge per molecular weight ratio of the CPM.

In some embodiments, the C_{H}3 domains are from human IgG and their charge engineering does not interfere with normal neonatal Fc receptor binding and cellular recycling. The C_{H}3 domains may be from human IgG and their charge-engineering modulates normal neonatal Fc receptor binding and cellular recycling in a manner that improves therapeutic efficacy of the protein entity.

Optionally, the target binding region comprises an antibody or an antibody fragment. The antibody fragment may be a single-chain antibody (scFv), an F(ab′)2 fragment, an Fab fragment, or an Fd fragment. In some embodiments, the protein entity comprises two distinct target binding regions so that the protein entity comprises a bispecific antibody.

In some embodiments, the target binding region comprises an antibody-mimic comprising a protein scaffold. Optionally, the Fv region is extended to have a second Fv region and spacer regions fused in sequence onto the L and H to create bispecificity on each chain. Alternatively, the target binding region comprises a DARPin polypeptide, an Adnectin polypeptide or an Anticalin polypeptide. In some embodiments, the target binding region comprises: a target binding scaffold from Src homology domains (e.g. SH2 or SH3 domains), PDZ domains, beta-lactamase, high affinity protease inhibitors, an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, or a C2 domain.

In some embodiments, the CPM binds to proteoglycans and promotes proteoglycan-mediated penetration into cells expressing the cell surface target. Optionally, the protein entity binds the cell surface target with at least approximately the same K_{D} or avidity as that of the target binding region alone. The protein entity may bind the cell surface target with at least 2-fold lower K_{D} or avidity as that of the target binding region alone. In some embodiments, the protein entity binds the cell surface target with a K_{D} or avidity less than or similar to that of the target binding region alone.

Optionally, the penetration of the protein entity into cells that express the cell surface target is increased relative to that of the target binding region alone. The targeting specificity of the protein entity may be increased relative to that of the CPM alone.

In some embodiments, the CPM has a net theoretical charge of from about +2 to about +15, such as from at about +3 to about +12. Optionally, the CPM has a charge per molecular weight ratio of less than 0.75, such as from about 0.2 to about 0.6. Further, the CPM may have a charge per molecular weight ratio of from greater than 0 to about 0.25.

The CPM may be a naturally occurring protein, such as a naturally occurring human protein. Alternatively, the CPM may be a domain of a naturally occurring protein. In certain embodiments, the naturally occurring protein is not the heavy chain of an Ig or is not a C_{H}3 domain of an Ig. In certain embodiments, the CPM is a naturally occurring human protein with an immunoglobulin domain, but which is not a portion of the Fc of an immunoglobulin.

In some embodiments, the CPM is a variant having at least two amino acid substitutions, additions, or deletions relative to a starting protein, and wherein the CPM has a greater net theoretical charge than the starting protein by at least +2 (e.g., is charge engineered). The starting protein may be a naturally occurring human protein. Optionally, the CPM is a variant having at least three, at least four, at least five, at least six, at least seven, at least 8, at least 9, or at least 10 amino acid substitutions relative to a starting protein. The CPM may be a variant having from 2-10 amino acid substitutions relative to a starting protein.

In some embodiments, the CPM has a greater net theoretical charge than the starting protein by at least +3, at least +4, at least +5, at least +6, at least +7, at least +8, at least +9, at least +10, at least +12, at least +14, at least +16, or at least +18. Optionally, the CPM has a greater net theoretical charge than the starting protein by from +3 to +15.

Optionally, the primary SR comprises a flexible peptide or polypeptide linker. The flexible peptide or polypeptide linker may comprise a plurality of glycine and serine residues. In some embodiments, the protein entity comprises a fusion protein comprising the target binding protein region interconnected to the CPM.

In certain embodiments, the cell surface target is not a sulfated proteoglycan. Optionally, the CPM exhibits binding for the cell surface that is blocked by soluble heparin sulfate or heparin sulfate proteoglycan (HSPG). The penetration of the protein entity into cells that express the cell surface target may be increased by at least 2-fold relative to that of the CPM alone.

In some embodiments, the protein entity further comprises a cargo region for delivery into a cell that expresses the cell surface target. The cargo region may be a polypeptide, a peptide, or a small molecule. Optionally, the cargo region comprises a small molecule, and wherein the small molecule is released as an active therapeutic agent after the protein entity is internalized into the target cell. The small molecule can be released by any of the following mechanisms: endogenous proteolytic enzymes, pH-induced cleavage in the endosome, or other intracellular mechanisms.

In some embodiments, the primary SR comprises a flexible linker comprising one or more sites for drug conjugation. For example, the one or more sites for drug conjugation may comprise more than one cysteine residues interposed between at least three or more non-reactive amino acid residues. Optionally, the SR comprises: (S₄G)₂-[Cys-(S₄G)]₄-(S₄G)₂

In some embodiments, the target binding region comprises a V_{H} and/or V_{L} of an Fab, and the CPM comprises a C_{H}1 domain and/or C_{L} domain of an immunoglobulin. Optionally, the target binding region comprises the V_{H} and/or V_{L} of an Fab, and the CPM comprises a C_{H}3 domain of an immunoglobulin. Further, the CPM may comprise a charge engineered variant of the CH1 and/or C_{H}L domains, or of the C_{H}3 domain.

In some embodiments, the CPM does not comprise all or a region of an immunoglobulin.

In some embodiments, the protein entity comprises a fusion protein. The fusion protein may be a single polypeptide chain. Optionally, the fusion protein is conjugated with one or more small molecules.

In another aspect, the disclosure provides a fusion protein comprising:

a target binding portion that binds a cell surface target with a dissociation constant (K_{D}) of greater than 0.01 nM or with an avidity of greater than 0.001 nM, and

a CPM that enhances penetration into cells;

wherein the CPM is a polypeptide having tertiary structure and a molecular weight of at least 4 kDa, wherein the CPM has surface positive charge and a net theoretical charge of less than +20;

wherein the cell surface target is distinct from that bound by the CPM;

and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone. In certain embodiments, effective penetration refers to the preferential enhancement of cell penetration of the protein entity as a function of expression of the cell surface target.

In another aspect, the disclosure provides a fusion protein comprising:

a target binding portion that binds a cell surface target with a dissociation constant (K_{D}) of greater than 0.01 nM or with an avidity of greater than 0.001 nM, and

a CPM that enhances penetration into cells;

wherein the CPM is a polypeptide having tertiary structure, a molecular weight of at least 4 kDa and a theoretical net charge of at least +5, wherein the CPM has surface positive charge and a charge per molecular weight ratio of less than 0.75;

wherein the cell surface target is distinct from that bound by the CPM;

and wherein the protein entity binds the cell surface target with sufficient affinity or avidity to effect penetration of the protein entity into cells that express the cell surface target, wherein penetration of the protein into the cells entity is increased relative to that of at least one of the target binding region alone or the CPM alone. In certain embodiments, effective penetration refers to the preferential enhancement of cell penetration of the protein entity as a function of expression of the cell surface target.

In another aspect, the disclosure provides a fusion protein comprising:

a first polypeptide portion comprising a target binding region that binds a cell surface target with a dissociation constant (K_{D}) of less than 1 μM or with an avidity of less than 1 μM, and

a second polypeptide portion comprising a CPM that enhances penetration into cells;

wherein the CPM is a polypeptide having tertiary structure and a molecular weight of at least 4 kDa, wherein the CPM has surface positive charge and a net theoretical charge of less than +20;

wherein the cell surface target is distinct from that bound by the CPM;

and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone. In certain embodiments, effective penetration refers to the preferential enhancement of cell penetration of the protein entity as a function of expression of the cell surface target.

An additional aspect of the present disclosure provides a fusion protein comprising: a first polypeptide portion comprising a target binding region that binds a cell surface target with a dissociation constant (K_{D}) of less than 1 μM or with an avidity of less than 1 μM, and a second polypeptide portion comprising a CPM that enhances penetration into cells; wherein the CPM is a polypeptide having tertiary structure and a molecular weight of at least 4 kDa and a theoretical net charge of at least +5, wherein the CPM has surface positive charge and a charge per molecular weight ratio of less than 0.75; wherein the cell surface target is distinct from that bound by the CPM; and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone. In certain embodiments, effective penetration refers to the preferential enhancement of cell penetration of the protein entity as a function of expression of the cell surface target.

In some embodiments, the CPM has a charge per molecular weight ratio of less than 0.75. Optionally, the CPM has a theoretical net charge less than +20.

The fusion protein may further comprise a third polypeptide region comprising a primary SR interconnecting the target binding region and the CPM. Optionally, an additional polypeptide region is connected to the CPM, the primary SR, or the target binding region.

In some embodiments, the fusion protein is further conjugated to a cargo region, wherein the cargo region is connected to at least one of the CPM, the primary SR, or the target binding region.

In some embodiments, the additional polypeptide region comprises an additional spacer region (SR) interposed between the CPM and the adjacent additional polypeptide region or the cargo region, and optionally followed by additional SR-polypeptide units, each additional SR having the same or a distinct sequence from the primary SR. Optionally, the primary SR comprises an immunoglobulin (Ig) region in a specific class of Ig heavy chain (H) that are genetically fused between the Fv region and C-terminal dimerization domains of each H chain. The Ig region may be an IgG, such as a human IgG.

In some embodiments, the fusion protein comprises a C-terminal dimerization domain of an immunoglobulin (Ig), and wherein the amino acid sequence of the C-terminal dimerization domain has been altered to increase surface positive charge and/or net positive charge to enhance penetration into cells. Optionally, the immunoglobulin is an IgG, preferably a human IgG, and the C-terminal dimerization domain comprises a pair of human C_{H}3 domains, of which the amino acid sequence of at least one domain has been altered to increase surface positive charge and/or net positive charge to enhance penetration into cells.

In some embodiments, the target binding region is a target-specific Fv region, comprising a light chain variable (V_{L}) domain mated with a heavy chain variable (V_{H}) domain. Optionally, the V_{H} and V_{L} domains are human, humanized, murine, chimeric, and wherein one or both of the V_{H} and V_{L} domains are optionally deimmunized.

In some embodiments, the CPM is N-terminal to the target binding region. Alternatively, the CPM may be C-terminal to the target binding region.

In a further aspect, the disclosure nucleic acid comprising a nucleotide sequence encoding the any of the fusion proteins described above.

In a related aspect, the disclosure provides a vector comprising any of the nucleic acid molecules described above.

In an additional aspect, the disclosure provides a host cell comprising any of the vectors described above.

A further aspect of the disclosure provides a method of making a fusion protein, comprising (i) providing any of the above host cells in culture media and culturing the host cell under suitable condition for expression of protein therefrom; and (ii) expressing the fusion protein.

In another aspect, the disclosure provides, a method of delivery into a cell, comprising providing any of the above protein entities or fusion proteins and contacting cells with the protein entity or the fusion protein. Optionally, the method comprises delivering a cargo region to a cell that expresses the cell surface target.

In an additional aspect, the disclosure provides a method of delivering a target binding region into cells, comprising providing any of the above protein entities or fusion proteins and administering said protein entity or said fusion protein to a subject in need thereof.

In a further aspect, the disclosure provides a method of delivering a cargo region into cells, comprising providing any of the above protein entities or fusion proteins, wherein said protein entity comprises the cargo region and administering said protein entity or said fusion protein to a subject in need thereof to deliver the protein entity into cells to deliver the cargo region.

In another aspect, the disclosure provides a method of enhancing penetration of a target binding region into cells, comprising providing any of the above protein entities or fusion proteins and contacting cells with said protein entity or said fusion protein or administering said protein entity or said fusion protein to a subject.

In a further aspect, the disclosure provides a method of enhancing penetration of a cargo region into cells, comprising providing any of the above protein entities or fusion proteins and administering said protein entity or said fusion protein to a subject in need thereof.

In certain embodiments of the foregoing aspects, the cargo region is a polypeptide, a peptide, or a small organic molecule. Optionally, the cargo region is an enzyme or a tumor suppressor protein. The cargo region may be a cytotoxic agent, such as auristatin, calicheamicin, maytansinoid, anthracycline, Pseudomonas exotoxin, Ricin toxin, or diphtheria toxin.

In a another aspect, the disclosure provides a method of enhancing penetration of a co-administered agents into cells, comprising providing any of the above protein entities or fusion proteins, administering said protein entity or said fusion protein to a subject in need thereof, and administering said agent to said subject, wherein the agent is administered at the same time, or, within the half-life of the protein entity or the agents, prior to or following administration of the protein entity or fusion protein.

In certain embodiments of the foregoing aspect, the agent is a polypeptide, a peptide, or a small organic molecule. Optionally, the agent is an enzyme or a tumor suppressor protein. The agent may be a cytotoxic agent, such as auristatin, calicheamicin, maytansinoid, anthracycline, Pseudomonas exotoxin, Ricin toxin, or diphtheria toxin.

In certain embodiments of any of the foregoing protein entity or fusion protein aspects, the cell surface target is expressed on cells of the immune system, such as B-cells.

In certain embodiments of any of the foregoing protein entity or fusion protein aspects, the cell surface target is expressed on cancer cells. Optionally, the cancer is selected from breast, kidney, colon, liver, lung, and ovarian. In some embodiments, the cell surface target is selected from a growth factor receptor, a GPCR, a lectin/sugar binding protein, a GPI-anchored protein, an integrin or a subunit thereof, a B cell receptor, a T cell receptor or a protein having an overexpressed extracellular domain present on the cell surface. The cell surface target may be selected from CD30, Her2, CD22, ENPP3, EGFR, CD20, CD52, CD 11a or alpha-integrin.

In some embodiments, the target binding region is selected from brentuximab, trastuzumab, inotuzumab, cetuximab, rituximab, alemtuzumab, efalizumab, or natalizumab, or an antigen binding fragment of any of the foregoing. Optionally, the target binding region is a scFv and the CPM is selected from Table [3].

The disclosure contemplates all combinations of any of the foregoing aspects and embodiments with each other, as well as combinations with any of the embodiments set forth in the detailed description and examples.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts design of Green Fluorescent Protein (GFP) charge series from five GFP charge variants. Each of the designed proteins is a variant of GFP with a particular theoretical net charge and a charge distribution, as depicted in the figure. These provide examples of charged protein moieties (CPMs).

FIG. 2 depicts Ni purification of +9GFP; the results of which were evaluated using Instant Blue coomassie staining.

FIG. 3 depicts Ni purification of +12GFPa-C6.5; the results of which were evaluated using Instant Blue coomassie staining. +12GFPa-C6.5 is an example of a protein entity of the present disclosure, and this protein entity comprises a target binding region that binds a cell surface target (in this case the target binding region is C6.5, a human single-chain Fv antibody (scFv) that binds to the Her2 extracellular domain) and a CPM (in this case +12GFPa).

FIG. 4 depicts cation exchange chromatography of +9GFP.

FIG. 5 depicts cation exchange chromatography of a +12GFPa-C6.5 fusion protein.

FIG. 6 depicts a gel analysis of the final product for +12GFPa-C6.5. This fusion protein was purified to at least 90% purity.

FIG. 7 depicts the results of serum stability evaluation for +15GFP-(S₄G)₆-C6.5-His₆ and C6.5-(S₄G)₆-+15GFP-His₆. Although presented in differing orientations, in each protein entity (in this case, fusion proteins), the target binding region is C6.5 and the CPM is +15GFP. In addition, each fusion protein includes a spacer region (in these cases, spacer region comprising serine and glycine residues) interconnecting the target binding region and the CPM, as well as an epitope tag (in this case, His₆ at the C-terminus).

FIG. 8 depicts flow cytometry analysis of Her2 levels on MDA-MB-468 and AU565 cells. The Her2 levels were measured by flow cytometry using an anti-Her2 antibody conjugated to allophycocynin (APC).

FIGS. 9A and 9B depict flow cytometry analysis for detecting GFP species in AU565 cells and in MDA-MD468 cells following 2 hour incubation of cells with the indicated fusion proteins.

FIG. 10A summarizes results from experiments using Her2^{high} AU565 cells indicating that charge can enhance penetration into cells in a manner that does not abrogate the binding specificity of a target-binding region to a cell surface receptor. Median fluorescence of flow cytometry data minus background fluorescence of untreated cells is depicted. For each charged series, the results for the GFP region alone (in the absence of fusion to a target binding region) are shown to the left.

FIG. 10B summarizes results from experiments using Her2^{low} MDA-MB-468 cells indicating that the charge of the CPM can enhance penetration in a manner that does not abrogate the binding specificity of a target-binding region to a cell surface receptor. The binding affinity of the target-binding region for its receptor affects the level of charge needed for internalization. Median fluorescence of flow cytometry data minus background fluorescence of untreated cells is depicted. For each charged series, the results for the GFP region alone (in the absence of fusion to a target binding region) are shown to the left.

FIG. 11A shows images of SKOV-3 cells (Her2^{high}) following treatment with 1 μM of protein for 1 hour. These images were taken to assess cellular uptake of these GFP-containing proteins by fluorescence microscopy. The images shown are an overlay of phase contrast and GFP fluorescence images.

FIG. 11B shows images of AU565 (Her2^{high}) and MDA-MB-468 cells (Her2^{Low}) following treatment with 1 μM of protein for 2 hours in serum-free media. These images were taken to assess cellular uptake of these GFP-containing proteins by fluorescence microscopy. The images shown are an overlay of phase contrast and GFP fluorescence images. The image of the control sfGFP-C6.5, which is not positively charged, was taken at 3× exposure over the others.

FIGS. 12A-12D depict a flow cytometry analysis of cellular uptake of the tested proteins. The Y-axis represents the level of Her2 expression, and the X-axis represents the level of GFP protein internalized in the cells. The median GFP fluorescence level of the two cell populations, AU565 (Her2^{high}) and MDA-MB-468 (Her2^{Low}), were quantified and compared in Tables 4 and 5.

FIGS. 13A-13J depict the median fluorescence value minus background-fluorescence of untreated cells (background adjusted fluorescence) (Y-axis) as a function of concentration (X-axis) for each of the tested proteins. Cellular uptake of the proteins was measured by GFP fluorescence. Her2 expression level was measured by using a Her2 antibody conjugated with allophycocyanin (APC). Gating was applied to the flow cytometry data to identify Her2^{low} versus Her2^{high} populations. The two concentration profiles represent the background adjusted fluorescence for the two cell populations present in the wells, i.e., the Her2^{high} cells (AU565) and the Her2^{Low} cells (MDA-MB-468). The Her2^{low} profiles (diamond) are indicative of the profile of charged GFP alone. The Her2^{high} profiles (square) are indicative of the profile of the charged GFP in combination with the target-binding region (C6.5). The data of sfGFP-C6.5 on the Her2^{high} cells reflects the profile of the c-terminal target-binding region (C6.5) by itself.

### DETAILED DESCRIPTION OF THE DISCLOSURE

### (i) Overview

The present disclosure provides a new class of penetration-enhanced targeted protein entities, also referred to as PETPs, PETP protein entities, and PETP entities, that are capable of binding to a specific cell surface target of interest and also has an enhanced cell-penetrating capability. The protein entities of the present disclosure comprise: (i) a target binding region, which is capable of binding a cell surface target at the cell surface (e.g., a cell surface receptor), and (ii) a charged protein moiety (CPM), which is capable of enhancing penetration into cells (e.g., enhancing, increasing, or promoting uptake into cells) and, when provided in the context of the target binding region, is capable of enhancing penetration into cells expressing the cell surface target. The target binding region and CPM represent the core of the PETP (the core of the protein entity). The protein entities of the present disclosure may also comprise an additional spacer region (SR) interconnecting the target binding region and the CPM. For example, the protein entities of the present disclosure comprise the general formula of:

[target binding region]−[spacer region]−[charged protein moiety].

The presence of the spacer region in the protein entities is optional. Since the protein entity may include additional modules and additional spacer regions, the spacer region interconnecting the target binding region and the CPM is generally referred to as the primary spacer region or primary SR.

As explained in further detail herein, the target binding region and CPM are the protein core of the PETP. However, this protein entity may comprise additional modules, including cargo regions, intended for delivery into cells. These cargo regions may be proteins, peptides, small molecules, and nucleic acids. In a particular embodiment, the protein entity is conjugated to a drug (e.g., a small molecule cargo) to facilitate delivery of the drug into cells in a targeted fashion. Without being bound by theory, the delivery of a cargo region, such as a small molecule drug or protein, may additionally have the benefit of improving effective concentration of the delivered protein or small molecule in the cytoplasm or nucleus of the cell into which it is delivered (e.g., delivery not only into the cell but also effectively to the nucleus or cytoplasm—decreased retention in endosome or other intracellular organelles).

The term “target-binding region,” as used herein, refers to a module of the PETP that is capable of binding a cell surface target at the cell surface with a certain level of specificity. The target binding region binds the cell surface target at the cell surface (e.g., via a domain that is extracellular). In the context of the present disclosure, the target binding region is also referred to as a “cell surface targeting region”. In other words, the function and activity of this module is to bind to a cell surface target via a domain that is extracellular, thereby contributing to enhanced penetration of the protein entity preferentially into particular cell types (e.g., cells expressing the cell surface target). Suitable target binding regions bind with a K_{D} and/or avidity within a certain range, as described herein (e.g., such as a K_{D} of greater than 0.01 nM and less than 1 μM or an avidity of greater than 0.001 nM and less than 1 μM). Without being bound by theory, suitable target binding regions should have sufficient affinity for their cell surface target to promote specific binding at the cell surface and to effectively promote localization of the protein entity to the surface of cells expressing the cell surface target. It should be noted that the presence of a target binding region does not mean that a protein entity of the disclosure will only localize and internalize to cells expressing the particular cell surface target. Rather, the presence of the target binding region enriches, generally significantly, the specificity with which the protein entity localizes to particular cells and tissue types (e.g., those expressing the cell surface target), and thus enhanced cell penetration is not ubiquitous. Rather, enhanced penetration is also enriched, generally significantly, for cell and tissue types expressing the cell surface target bound at the cell surface by the target binding region. Generally, the protein entities of the disclosure lead to preferentially enhanced cell penetration as a function of both the target binding regions and the CPM.

In certain embodiments, uptake of the protein entity is, at least, 1.5, 2, 2.5, 3, 3.5, 4, 5, or greater than 5 times higher into cells that express the cell surface target versus into cells that do not express the cell surface target. In other words, in certain embodiments, cell penetration of the protein entity is enhanced at least 1.5, 2, 2.5, 3, 3.5, 4, 5, or greater than 5 times (e.g., fold) when evaluating cells that express the cell surface target at the cell surface versus cells that do not express the cell surface target at the cell surface. In certain embodiments, cell penetration of the protein entity is enhanced about 4, about 5, about 8 or about 16 fold when evaluating cells that express the cell surface target at the cell surface versus cells that do not express the cell surface target at the cell surface. In certain embodiment, cell penetration of the protein entity is enhanced at least 8 fold or at least 16 fold when evaluating cells that express the cell surface target at the cell surface versus cells that do not express the cell surface target at the cell surface. This is in sharp contrast to cell uptake based on the activity of the CPM alone, and is in particularly sharp contrast to the activity of supercharged proteins with a higher charge per molecular weight ratio and/or higher net charge. This illustrates the manner in which the target binding region is a cell surface targeting region and contributes to enhanced localization of the protein entity at the surface of particular cell types (e.g., cells expressing the cell surface target). In other words, preferentially enhanced cell penetration is provided by the protein entities of the disclosure.

Examples of target-binding regions that can be used in the present disclosure as regions that specifically bind at the cell surface to cell surface targets include, without limitation, antibodies, antibody fragments (e.g., antigen binding fragments, such as single-chain Fv or scFv binding sites, other engineered formats of the antibody binding site (comprising intact Fv regions or V_{H} and/or V_{L} domains that specifically associate with one or more targets), or antibody binding site mimics, including single-scaffold binders, that are capable of specifically binding a cell surface protein target (e.g., binds with affinity, avidity, and specificity distinct from non-specific interactions; suitable ranges are described herein). Additional features of target binding regions for use in the protein entities and methods of the present disclosure are described herein. Further, the disclosure provides non-limiting examples of target binding regions, as well as suitable cell surface targets that are specifically bound by a suitable target binding region. Examples of categories of cell surface targets are described herein. By way of example, they include growth factor receptors.

The term “charged protein moiety,” as used herein, refers to a positively charged molecule that is capable of penetrating cells and enhancing penetration into cells (e.g., enhancing uptake). When used as a module of a PETP, in accordance with the present disclosure, the CPM is capable of promoting or enhancing the penetration of the protein entities into cells without disrupting the ability of the target binding region to bind its cell surface target at the cell surface. As such, in the context of a protein entity, the CPM acts in a concerted manner with the target binding region to promote cell targeted internalization. In other words, the activity of the protein entity is a function of both the specific cell targeting of the target binding region and the penetration activity of the CPM, such that, penetration of the protein entity is enhanced as a function of both the activity of the cell targeting region (e.g., binding to a cell surface target at the cell surface) and the CPM. In certain embodiments, cell penetration of the protein entity is at least 1.5, 2. 2.5, 3, 3.5, 4, 4.5, 5, or greater than 5 fold higher into cell that express the cell surface target relative to cells that do not express the cell surface target or that only express the cell surface target at very low levels. This is an example of increased specificity where the protein entity has cell penetration ability with improved cell specificity due to its association with the cell targeting region relative to that of the CPM. Regardless of whether the foregoing improvement in specificity is achieved or evaluated, in the presence of the target binding region, the protein entity binds the cell surface target with sufficient affinity or avidity to effect penetration of the protein entity into cells that express the cell surface target. In other words, penetration into those particular cells (e.g., cells that express the cell surface target on the cell surface) is a function of both the CPM and the target binding region.

A CPM, in accordance with the present disclosure, has surface positive charge, net positive charge, and tertiary structure (e.g., a globular protein). Additionally, a CPM has a molecular weight of at least 4 kDa. Additional features of a CPM for use in the protein entities and methods of the disclosure are provided herein. Further, the disclosure provides non-limiting examples of CPMs.

The term “spacer region,” (“SR”) as used herein, refers to a linking region interconnecting two modules, such as the target-binding region and the CPM. The SR may be a peptide or polypeptide linking region or the SR may be a chemical linker. The term primary spacer region is generally used to refer to the linking sequence, when present, that interconnects the target binding region and the CPM. However, the protein entity may include additional SRs interconnecting other regions of the protein entity. When more than one SR is present, the length and sequence of each SR is independently selected. As detailed below, in certain embodiments, the primary SR is a polypeptide or peptide linking region, such as a flexible polypeptide or peptide linking region. Regardless of whether the primary SR is a polypeptide or peptide linking region, the nature of any additional SRs are independently selected. In certain embodiments, protein modules are connected to the protein entity directly or via a polypeptide or peptide linker, but small molecule (e.g., drugs) are connected to the protein entity via chemical conjugation, such as through conjugation via a reactive cysteine or lysine residue.

The term “protein entity of the disclosure” is used to refer to a protein entity or Protein-Enhanced Targeted Protein (PETP) comprising at least one target-binding region, and at least one CPM and optionally at least one SR. The target binding region and CPM are the core of the protein entity, and each can be considered as a module of the protein entity. The target-binding region, which may be an antibody, an antibody fragment (e.g., an antigen binding fragment such as a single chain Fv), or an antibody-mimic, binds a target expressed on the cell surface of cells, and the CPM functions to facilitate delivery of the protein entity into such cells (e.g., the CPM promotes or enhances penetration; the CPM promotes cell uptake). In certain embodiments, the target binding region and the CPM are heterologous regions with respect to each other. In other words, the target binding region and CPM are not naturally found contiguous to each other and/or are not regions of the same naturally occurring protein. In certain embodiments, the target binding region and CPM are regions of the same naturally occurring protein but, in the context of the protein entity, the regions are not configured or provided in the same way as found in the naturally occurring protein. For example, the target binding region and CPM may be connected via a SR that is different from the amino acid sequence that is contiguous to these regions in their naturally occurring context. In other embodiments, the target binding region and CPM may be domains of the same or a highly related protein, optionally, with one or more amino acid alterations in one or both regions relative to a starting or native protein. The target binding region and CPM may be connected via an SR that is different from the amino acid sequence that is contiguous to these regions in their naturally occurring context or a SR differs. In certain embodiments, the protein entities of the disclosure further comprise a primary spacer region (SR) that interconnects the target binding region and the CPM. The core protein entity, in the presence or absence of a primary SR, may further comprise additional modules (which are optionally connected to the protein entity directly or indirectly). Suitable additional modules include cargo regions, such as proteins, peptides, small molecules (including therapeutic or cytotoxic drugs), and nucleic acids. It should be noted that the protein entity may include non-protein components, including non-protein linking regions and appended small molecules.

In the context of a protein entity, the activity of the protein entity is a function of both the specific cell targeting of the target binding region and the penetration activity of the CPM, such that, penetration of the protein entity is enhanced as a function of both the activity of the cell targeting region (e.g., binding to a cell surface target at the cell surface) and the CPM. In certain embodiments, cell penetration of the protein entity is at least 1.5, 2. 2.5, 3, 3.5, 4, 4.5, 5, or greater than 5 fold higher into cell that express the cell surface target relative to cells that do not express the cell surface target or that only express the cell surface target at very low levels. This is an example of increased specificity where the protein entity has cell penetration ability with improved cell specificity due to its association with the cell targeting region relative to that of the CPM. Regardless of whether the foregoing improvement in specificity is achieved or evaluated, in the presence of the target binding region, the protein entity binds the cell surface target with sufficient affinity or avidity to effect penetration of the protein entity into cells that express the cell surface target. In other words, penetration into those particular cells (e.g., cells that express the cell surface target on the cell surface) is a function of both the CPM and the target binding region.

Also provided are nucleic acid molecules encoding such protein entities or encoding the target binding region, the SR, or the CPM portion of such protein entities, as well as methods of making and using such protein entities.

The present disclosure is based on the discovery that combining in a protein entity the internalization abilities of CPMs (including naturally occurring and charge-engineered proteins) with the cell surface targeting abilities of a target-binding region (e.g., an antibody, an antibody fragment (e.g., an antigen binding fragment such as an scFv), or an antibody mimic that specifically binds a cell surface target at the cell surface) achieves a better balancing of two functions: cell targeting and enhanced cell penetration. The present disclosure provides a solution to solve the current problem of imbalance between the two functions. If there is too much non-specific penetration, the target-binding region may not achieve broad tissue distribution, and/or will not necessarily effectively localize to a cell or tissue type of interest (e.g., tissue distribution may be ubiquitous). This may increase the amount of therapeutic that must be delivered to get sufficient protein to a cell or tissue of interest, or may increase the risk of off-target effects due to lack of targeting. On the other hand, if there is too little penetration or the binding between the target-binding region and its cell surface target is not strong enough, the protein entity may not penetrate into cells before the target-binding portion disengages from its cell surface target. The present disclosure provides protein entities that are capable of achieving a balance between the cell penetration and the target binding functions, and thus provides for therapeutic developments. Thus, not only do the protein entities provide targeting to a cell type of interest, they also demonstrate the benefit of balancing the cell penetration activity of the CPM so that it does not overwhelm the ability to target particular cell types. In other words, the activity of the protein entity is a function of both the specific cell targeting of the target binding region and the penetration activity of the CPM, such that, penetration of the protein entity is enhanced as a function of both the activity of the cell targeting region (e.g., binding to a cell surface target at the cell surface) and the CPM. In certain embodiments, cell penetration of the protein entity is at least 1.5, 2. 2.5, 3, 3.5, 4, 4.5, 5, or greater than 5 fold higher into cell that express the cell surface target relative to cells that do not express the cell surface target or that only express the cell surface target at very low levels. This is an example of increased specificity where the protein entity has cell penetration ability with improved cell specificity due to its association with the cell targeting region relative to that of the CPM. Regardless of whether the foregoing improvement in specificity is achieved or evaluated, in the presence of the target binding region, the protein entity binds the cell surface target with sufficient affinity or avidity to effect penetration of the protein entity into cells that express the cell surface target. In other words, penetration into those particular cells (e.g., cells that express the cell surface target on the cell surface) is a function of both the CPM and the target binding region.

Without being bound by theory, the present disclosure provides a protein entity, also known as a PETP, comprising a target-binding region and a charged protein moiety. Such protein entities retain the target binding function of the target binding region, and bind cells that express the cell surface target with sufficient affinity or avidity for the target-binding region to promote localization of a protein entity to a subset of cells or tissues (e.g., to promote localization that is not ubiquitous). Furthermore, the protein entities also penetrate into cells that express the cell surface target as a function of the activity of the CPM. The target-binding region is capable of guiding the protein entity into cells with specificity, such that enhanced cell penetration is not ubiquitous or limited to the site of delivery, but rather, is enhanced preferentially to cells that express the cell surface target following binding of the target binding region to its cell surface target. As a result of the joint activity of the target binding region and the CPM, the present disclosure provides a novel delivery platform for promoting or enhancing penetration into cells that express a cell surface target specifically bound by the target binding region present as part of the protein entity. This platform can be used, for example, to promote targeted cell penetration, to deliver a CPM and/or target binding region into a cell, and to deliver a cargo region, such as a therapeutic or cytotoxic agent, attached to the protein entity.

Features of this interaction and the various components of protein entities of the disclosure are described herein. The CPM is capable of promoting or enhancing penetration into cells (e.g., promoting or enhancing uptake into cells; promoting or enhancing delivery across the cell membrane). Without being bound by theory, this activity of the CPM may be mediated by binding to proteoglycans (e.g., proteoglycan-mediated internalization). In the context of the present disclosure, the CPM is specifically (although not necessarily exclusively) directed to cells that express the cell surface target bound by the target binding region of the protein entity, and thus, the CPM promotes or enhances penetration into those cells expressing the cell surface target. As a result, the penetration of the protein entity is increased relative to that of the target binding region alone or the CPM alone. Moreover, the specificity of cell penetration increases because it is not driven entirely by the charge characteristics of the CPM. Of course, the localization and penetration of the protein entity is not exclusive to cells expressing the cell surface target. However, localization and penetration is non-ubiquitous, not limited to the immediate site of administration, and enriched (including significantly enriched) relative to localization and internalization of the CPM alone.

The protein entities of the present disclosure may also be conjugated with a cargo molecule. Examples of cargo molecules include, without limitation, polypeptides, peptides, small organic molecules (such as cytotoxic drugs), chemotherapeutic agents, RNA- or DNA-based drugs. These protein entities facilitate targeted delivery and penetration of the cargo into the target cells. Thus, the protein entities of the present disclosure are useful for delivering the cargo into cells for treating disease, correcting an intracellular protein deficiency, to study cell behavior and dysfunction, to develop therapies, and the like.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the terms “associated with,” or “associate by” when used with respect to the target-binding region and the CPM of a protein entity of the disclosure, means that these portions are physically associated or connected with one another, either directly or via one or more additional moieties, including moieties that serve as a linking agent (e.g., a spacer region), to form a structure that binds the cell surface target with sufficient affinity or avidity to effect internalization of the protein entity into cells that express the cell surface target. The association may be via non-covalent interactions and/or via covalent interconnections. The protein entity may be a single polypeptide chain, or it may be composed of more than one polypeptide chain. In either case, the association among any of the components of a protein entity may be direct or via a spacer region or via additional polypeptide sequence. Moreover, the association may be disruptable, such as by cleavage of a spacer region that interconnects the portions of the protein entity. In certain embodiments, such cleavage may occur following internalization into a cell, and the cleavage may be induced by the pH environment of the endosome. The protein entity may be a fusion protein in which the target-binding region and the CPM are connected by a peptide bond as a fusion protein, either directly or via a spacer region or other additional polypeptide sequence. In certain embodiments, the target-binding region binds to a cell surface target (e.g., a target expressed or present on the cell surface) that is distinct from a cell surface target that is bound by the CPM present in the protein entity.

As used herein, the term “charge engineering” or “charge engineered” refers to any modification of a protein, the primary purpose of which is to increase the net charge or the surface charge of the protein to make that protein suitable for or to improve its suitability for use as a CPM. Modifications include, but are not limited to, amino acid substitution, addition, or deletion (collectively “alteration”). When more than one amino acid alteration is made, each alteration is independently selected. Alternatively, two or more residues may be chosen based on their spatial relationship to each other. In certain embodiments, charge engineering comprises at least one, at least two, at least three, at least four, at least five, at least six, at least seven, at least eight, at least nine, or at least ten amino acid substitutions relative to a starting sequence. In certain embodiments, the charge engineering results in an increase in net positive charge, in comparison to the starting sequence, of at least +1, at least +2, at least +3, at least +4, at least +5, at least +6, at least +7, at least +8, at least +9, at least +10, at least +12, at least +14, at least +15, at least +16, at least +18, at least +20, at least +21, or at least +22. In certain embodiments, the starting sequence is negatively charged and through charge engineering a positively charged protein is generated. When multiple alterations are made, each is independently selected. In other words, for each alteration, an independent decision is made regarding (i) whether the alteration is a substitution, addition, or deletion and (ii) if a substitution, what residue is substituted. In certain embodiments, at each position, the substitution is independently selected to replace a residue with a His, Arg, or Lys. In certain embodiments, at each position, the substitution is independently selected to replace a negatively charged residue with an uncharged residue or a positively charged residue.

### (ii) Target-Binding Region

The term “target-binding region” as used herein, refers to a module of the PETP that is capable of binding a cell surface target with a certain level of specificity. “Cell surface target binding region” may similarly be used to describe this feature. Suitable target binding regions bind with a K_{D} and/or avidity within a certain range, as described herein (e.g., such as a K_{D} of greater than 0.01 nM and less than 1 μM or an avidity of greater than 0.001 nM and less than 1 μM). Without being bound by theory, suitable target binding regions should have sufficient affinity for their cell surface target to promote specific binding and to effectively promote localization of the protein entity to cells expressing the cell surface target. It should be noted that the presence of a target binding region does not mean that a protein entity of the disclosure will only localize and internalize to cells expressing the particular cell surface target. Rather, the presence of the target binding region enriches, generally significantly, the specificity with which the protein entity localizes to particular cells and tissue types (e.g., those expressing the cell surface target at the cell surface), and thus internalization is not ubiquitous. Rather, internalization is also enriched, generally significantly, for cell and tissue types expressing the cell surface target bound by the target binding region relative to internalization into cells that do not express the cell surface target. In certain embodiments, internalization of the protein entity is, at least, 1.5, 2, 2.5, 3, 3.5, 4, 5, or greater than 5 times higher into cells that express the cell surface target versus into cells that do not express the cell surface target. In certain embodiments, internalization of the protein entity is, at least, 8, 10, 16, or greater than 16 times higher into cells that express the cell surface target versus into cells that do not express the cell surface target. In certain embodiments, internalization of the protein entity is, about 5, about 8, about 10, or about 16 times (fold) higher into cells that express the cell surface target versus into cells that do not express the cell surface target. Further structural and functional features of a target binding region are described below.

Initially, it should be noted that suitable protein entities reflect a balance between the activity of the cell targeting region (e.g., specific binding to the cell surface target at the cell surface) and that of the CPM (promoting or enhancing internalization). Thus, the charge and charge distribution of the CPM is balanced against the K_{D} and affinity of the target binding region. Using the teachings of the present disclosure, one of skill in the art can select a CPM suitable for pairing with a particular target binding region, and vice versa. As detailed below, a relationship exists between the desired affinity and or K_{D}/avidity of the target binding region and charge characteristics (e.g., net positive charge, charge per molecular weight ratio and/or surface positive charge) of the CPM. By selecting these modules of the protein entity to optimize the balance of the functions of these modules, protein entities of the disclosure having cell targeting and enhanced internalization characteristics are obtained.

Target binding regions for use herein bind to a cell surface target at the cell surface, as defined below, and suitable target binding regions have particular structural and functional features. Before describing the structural and function features of suitable target binding regions, we first describe the types of moieties that are suitable for use as a target binding region. Any such class of target binding compounds may be used as the target binding region of a PETP. These constitute a first module of the PETP. Exemplary classes of target-binding regions include antibodies, antibody fragments (e.g., antigen binding fragments, such as a single chain Fv), and antibody mimics that bind to a cell surface target. Regardless of the particular class of target binding region, the disclosure contemplates that any such class of target binding region may be used in combination with any class of CPM, and optionally with one or more additional regions, such as SRs and cargo regions. The protein entity of the disclosure has an increased targeting specificity as a function of the presence of the target-binding region in the protein entity. In certain embodiments, the targeting specificity of the protein entity is increased relative to that of the CPM alone. In certain embodiments, the targeting specificity of the protein entity is increased relative to that of the target binding region alone. In the context of the present disclosure, the binding of the target binding region to the cell surface target at the cell surface contributes (e.g., helps effect) cell penetration into cells expressing that cell surface target. In other words, the binding of the protein entity at the cell surface via the target binding region influences penetration (e.g., uptake) into those cells.

The target binding region may be monovalent, divalent, multivalent (such as bispecific IgG-scFv fusions (Coloma and Morrison, 1997) and SEEDbodies (Davis, et al., PEDS, 2010)), monospecific, bispecific, multispecific or polyspecific binders. For example, the target binding region may be a single domain binding protein comprising a V_{H} or V_{L} domain, multiples thereof, a single domain antibody, a humanized VHH camelid binding domain, a single scaffold binding protein (for example, affibody, an adnectin, or a DARPin). The target binding region may comprise fused subdomains, a highly stable Fv region, or stabilized forms of the antibody binding site (e.g., a single-chain Fv, a disulfide stabilized Fv (dsFv)), a diabody, a single chain diabody, tandem scFv repeats of the same or distinct scFv, an Fab with or without an interchain disulfide, a single chain Fab, a cloned naturally-occurring human antibody, or a recombinant humanized or human analogue of binding fragments or domains derived from antibody domains of non-human origin or a combination of any of the above-described binding molecules. The target binding region may also comprise a non-antibody antibody binding site.

The target binding region of the present disclosure may comprise more than one subcomponents and each subcomponent is an antibody, antibody fragment, such as an scFv, or an antibody mimic that binds to a cell surface target. The multiple-component target binding region may comprise a linker interconnecting at least two subcomponents of a target-binding region. The target binding region may also comprise linker chains bridging at least two subunits to a target-binding region, of which at least one subunit needs to be in the fusion protein of this invention (see general modular design 1), including fusion to either (or both) the V_{H} or V_{L} domain within a disulfide-stabilized Fv, dsFv, or as a fusion partner with or within the L and/or H chains of IgG or any of the chains or domains in any class or IgA, IgM, other members of the Ig superfamily, or conjugates thereof, or engineered multivalent binders such as the bispecific IgG-scFv fusions (Coloma and Morrison, 1997), SEEDbodies (Davis, et al., PEDS, 2010), and so forth.

In certain embodiments, the target-binding region is an antibody, an antibody fragment (e.g., an antigen binding fragment), or an antibody mimic molecule that specifically binds to a cell surface target. An antibody-mimic molecule is also referred to as an antibody-like molecule. An antibody-mimic binds to a cell surface target, but binding is mediated by binding units other than antigen binding portions comprising at least a variable heavy or variable light chain of an antibody. Thus, in an antibody mimic, binding to a cell surface target is mediated by a different antigen-binding unit, such as a single-scaffold binder protein or Ig superfamily scaffold binder protein or other engineered protein binding units. Numerous categories of antibody-mimics are well known in the art and are described in further detail below.

In certain embodiments, the target-binding region is an adhesin molecule. In certain embodiments, the term “adhesin” refers to a chimeric molecule which combines the “binding domain” (e.g., the extracellular domain) of a heterologous “adhesion” protein (e.g., a receptor, ligand, or enzyme) with an immunoglobulin sequence. In certain embodiments, the immunoglobulin sequence is an immunoglobulin effector or constant domain (e.g., all or a portion of an Fc domain; one or more of an Ig C_{L}1, hinge, C_{H}1, C_{H}2, or C_{H}3). Structurally, the immunoadhesins comprise a fusion of the adhesion amino acid sequence with the desired binding specificity which is other than the antigen recognition and binding site of an antibody (i.e., is “heterologous”) and an immunoglobulin effector or constant domain sequence. The immunoglobulin constant domain sequence in the adhesin molecule may be obtained from any immunoglobulin, such as IgG1, IgG2, IgG3, or IgG4 subtypes, IgA, IgE, IgD or IgM. Such adhesin molecule has the ability of specifically binding to the target. Numerous categories of such polypeptides (e.g., adhesin molecules) are well known in the art and are described in further detail below.

In certain embodiments, a protein entity of the disclosure comprises a target-binding region, wherein the target-binding region is an antibody or an antibody mimic molecule that binds to a cell surface target molecule. In certain embodiments, a protein entity of the disclosure comprises a target binding region, wherein the target-binding region is an antibody-mimic (e.g., a protein comprising a protein scaffold or other binding unit that binds to a target). In certain embodiments, a protein entity of the disclosure comprises a target-binding region, wherein the target-binding region comprises a ligand or a receptor-binding domain of the ligand. In certain embodiments, a protein entity of the disclosure comprises a target-binding region, wherein the target-binding region comprises a receptor, or a ligand-binding domain of the receptor, or an extracellular domain of the receptor.

In certain embodiments, a target-binding region is an antibody-mimic comprising a protein scaffold. Scaffold-based target binding regions have positioning or structural components and target-contacting components in which the target contacting residues are largely concentrated. Thus, in an embodiment, a scaffold-based target-binding region comprises a scaffold comprising two types of regions, structural and target contacting. The target contacting region shows more variability than does the structural region when a scaffold-based target-binding region to a first target is compared with a scaffold-based target-binding region of a second target. The structural region tends to be more conserved across target binding regions that bind different targets. This is analogous to the CDRs and framework regions of antibodies. In the case of an Anticalin®, the first class corresponds to the loops, and the second class corresponds to the anti-parallel strands.

In certain embodiments the target-binding region is a subunit-based target-binding region. These target binding regions are based on an assembly of subunits which provide distributed points of contact with the cell surface target that form a domain that binds with high affinity or avidity to the target (e.g. as seen with DARPins).

Regardless of the particular category of target binding region selected, the target binding region binds a cell surface target. In the context of a protein entity, the target binding region binds the cell surface target at the cell surface, and thus contributes to penetration of the protein entity into cells.

In certain embodiments a target-binding region for use as part of a protein entity of the disclosure has a molecular weight of 5-250, 10-200, 5-15, 10-30, 15-30, 20-25 kD, 50-100 kD, or 50-75 kD. Target binding regions can comprise one or more polypeptide chains, or one, two, or more binding domains. In certain embodiments, the foregoing molecular weights refer to one polypeptide chain of the target binding region. In other embodiments, the foregoing molecular weights refer to the target binding region, as a whole (e.g., if the target binding region comprises two polypeptide chains, then the molecular weight is the combined MW of the two chains).

Target binding regions can be antibody-based or non-antibody-based.

The single-chain Fv is based on V_{H} and V_{L} domains that can be derived from a naive or immunized human V-gene antibody library or from B-cell repertoire cloning. The scFv is patentably distinct from antibodies, although the V_{H} and V_{L} genes of scFv that are desirable binders may be reconfigured in appropriate plasmids for expression in plants, yeast, special strains of E. coli, CHO or other standard cell lines, including mammalian cell expression systems.

Target binding regions suitable for use in the compositions and methods featured in the disclosure include antibody molecules, such as full-length antibodies and antigen-binding fragments thereof, and single domain antibodies, such as camelids. In certain embodiments, the target binding region is a single chain Fv comprising a V_{H} domain and V_{L} domain connected via a linker, such as a flexible polypeptide linker.

Regardless of the particular category of target binding region selected, the target binding region binds a cell surface target. In the context of a protein entity, the target binding region binds the cell surface target at the cell surface, and thus localizes the protein entity at specific cells of interest (e.g., helps effect penetration of the protein entity into cells that express the cell surface target on the cell surface).

Other suitable target binding regions include polypeptides engineered to contain a scaffold protein, such as a DARPin or an Anticalin®. These are exemplary of antibody-mimic moieties that, in the context of the disclosure, may be connected (e.g., combined or fused) with a CPM to promote internalization of the protein entity into cells that express a cell surface target at the cell surface, to which the target-binding region binds. Regardless of the particular category of target binding region selected, the target binding region binds a cell surface target. In the context of a protein entity, the target binding region binds the cell surface target at the cell surface, and thus localizes the protein entity at specific cells of interest (e.g., helps effect penetration of the protein entity into cells that express the cell surface target on the cell surface).

Antibody Molecules

As used herein, the term “antibody” or “antibody molecule” refers to a protein that includes sufficient sequence (e.g., antibody variable region sequence) to mediate binding to a cell surface target, and in embodiments, includes at least one immunoglobulin variable region (the Fv) or antigen binding domain thereof (V_{H} or V_{L}), or an antibody fragment thereof (an Fab), or recombinant species that comprise the V_{H} and V_{L} domains, such as an scFv, disulfide stabilized Fv (dsFv), an scFab, a diabody or single-chain diabody, exemplary of other binding formats.

An antibody molecule can be, for example, a full-length, mature antibody, or an antigen binding fragment thereof. An antibody molecule, also known as an antibody or an immunoglobulin, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens). The antibody molecule can be a single domain antibody, e.g., a nanobody, such as a camelid, or a llama- or alpaca-derived single domain antibody, or a shark antibody (IgNAR). The single domain antibody comprises, e.g., only a variable heavy domain (VHH). An antibody molecule can also be a genetically engineered single domain antibody. Typically, the antibody molecule is a human, humanized, chimeric, camelid, shark or in vitro generated antibody.

Examples of fragments include (i) an Fab fragment having a VL, VH, constant light chain domain (CL) and constant heavy chain domain 1 (CH1) domains; (ii) an Fd fragment having VH and CH1 domains; (iii) an Fv fragment having VL and VH domains of a single antibody; (iv) a dAb fragment (Ward, E. S. et al., Nature 341, 544-546 (1989); McCafferty et al (1990) Nature, 348, 552-55; and Holt et al (2003) Trends in Biotechnology 21, 484-490), having a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide spacer region which allows the two domains to associate to form an antigen binding site (Bird et al, Science, 242, 423-426, 1988 and Huston et al, PNAS USA, 85, 5879-5883, 1988) (viii) bispecific single chain Fv dimers (for example as disclosed in WO 1993/011161) and (ix) “diabodies”, multivalent or multispecific fragments constructed by gene fusion (for example as disclosed in WO94/13804 and Holliger, P. et al, Proc. Natl. Acad. Sci. USA 90 6444-6448, 1993). Fv, scFv or diabody molecules may be stabilized by the incorporation of disulphide bridges linking the VH and VL domains (Reiter, Y. et al, Nature Biotech, 14, 1239-1245, 1996). Minibodies comprising a scFv joined to a CH3 domain may also be made (Hu, S. et al, Cancer Res., 56, 3055-3061, 1996). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group. These antibody fragments are obtained using conventional techniques known to those with skill in the art, and the fragments are screened for utility in the same manner as are intact antibodies. Suitable fragments may, in certain embodiments, be obtained from human or rodent antibodies.

The term “antibody molecule” includes intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function). In certain embodiments, antibodies for use in the present disclosure are labeled, modified to increase half-life, and the like. For example, in certain embodiments, the antibody is chemically modified, such as by PEGylation, or by incorporation in a liposome.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, light chains devoid of heavy chains, single domain antibodies derived from conventional 4-chain antibodies, and engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the disclosure, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies derived from a variable region of NAR (“IgNARs”) are described in WO 03/014161 and Streltsov (2005) Protein Sci. 14:2901-2909. According to another aspect, a single domain antibody is a naturally occurring single domain antibody known as a heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; and such VHHs are within the scope of the disclosure.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Each VH and VL typically includes three CDRs and four FRs, arranged from amino-terminus to carboxy-terminus in the following order: FR1 CDR1, FR2, CDR2, FR3, CDR3, FR4.

The VH or VL chain of the antibody molecule can further include all or part of a heavy or light chain constant region, to thereby form a heavy or light immunoglobulin chain, respectively. In one embodiment, the antibody molecule is a tetramer of two heavy immunoglobulin chains and two light immunoglobulin chains. The heavy and light immunoglobulin chains can be connected by disulfide bonds. The heavy chain constant region typically includes three constant domains, CH1, CH2 and CH3. The light chain constant region typically includes a CL domain. The variable region of the heavy and light chains contains a binding domain that interacts with an antigen. The constant regions of the antibody molecules typically mediate the binding of the antibody to host tissues or factors, including various cells of the immune system (e.g., effector cells) and the first component (Clq) of the classical complement system.

The term “immunoglobulin” comprises various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the present disclosure. All immunoglobulin classes are also within the scope of the present disclosure. Light chains are classified as either kappa or lambda (κ, λ). Each heavy chain class may be bound with either a kappa or lambda light chain.

The term “antigen-binding fragment” refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment having VL, VH, CL and CH1 domains; (ii) a F(ab′)₂ fragment, a bivalent fragment including two Fab fragments linked by a disulfide bridge at the hinge region; (iii) an Fd fragment having VH and CH1 domains; (iv) an Fv fragment having VL and VH domains of a single arm of an antibody, (v) a dAb fragment (Ward et al., (1989) Nature 341:544-546), which has a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic spacer region that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The term “antigen-binding site” refers to the part of an antibody molecule that comprises determinants that form an interface that binds to a target antigen, or an epitope thereof. With respect to proteins (or protein mimetics), the antigen-binding site typically includes one or more loops (of at least four amino acids or amino acid mimics) that form an interface that binds to the target antigen or epitope thereof. Typically, the antigen-binding site of an antibody molecule includes at least one or two CDRs, or more typically at least three, four, five, or six CDRs.

Regardless of the type of antibody used, in certain embodiments, the antibody may comprise replacing one or more amino acid residue(s) with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. (Voet & Voet, Biochemistry, 2nd Edition, (Wiley) 1995). Those amino acid residues that are derivatised at their N-alpha position will only be located at the N-terminus of an amino-acid sequence. Normally, an amino acid is an L-amino acid, but it may be a D-amino acid. Alteration may therefore comprise modifying an L-amino acid into, or replacing it with, a D-amino acid. Methylated, acetylated and/or phosphorylated forms of amino acids are also known, and amino acids in the present disclosure may be subject to such modification. Additionally, the derivative can contain one or more non-natural or unusual amino acids by using the Ambrx ReCODE.™ technology (see, e.g., Wolfson, 2006, Chem. Biol. 13(10):1011-2).

In certain embodiments, the antibodies used in the claimed methods are generated using random mutagenesis of one or more selected VH and/or VL genes to generate mutations within the entire variable domain. Such a technique is described by Gram et al., 1992, Proc. Natl. Acad. Sci., USA, 89:3576-3580 who used error-prone PCR. In some embodiments one or two amino acid substitutions are made within an entire variable domain or set of CDRs.

Another method that may be used is to direct mutagenesis to CDR regions of VH or VL genes. Such techniques are disclosed by Barbas et al., 1994, Proc. Natl. Acad. Sci., USA, 91:3809-3813 and Schier et al., 1996, J. Mol. Biol. 263:551-567.

Regardless of the particular category of target binding region selected, the target binding region binds a cell surface target. In the context of a protein entity, the target binding region binds the cell surface target at the cell surface, and thus localizes the protein entity at specific cells of interest (e.g., helps effect penetration of the protein entity into cells that express the cell surface target on the cell surface).

Preparation of Antibodies

Suitable antibodies for use as a target-binding region can be prepared using methods well known in the art. For example, antibodies can be generated recombinantly, made using phage display, produced using hybridoma technology, etc. Non-limiting examples of techniques are described briefly below.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Köhler and Milstein, Nature, 256:495-497, 1975.

Monoclonal antibodies can be obtained, for example, from a cell obtained from an animal immunized against the target antigen, or one of its fragments. Suitable fragments and peptides or polypeptides comprising them may be used to immunize animals to generate antibodies against the target antigen.

The monoclonal antibodies can, for example, be purified on an affinity column on which the target antigen or one of its fragments containing the epitope recognized by said monoclonal antibodies, has previously been immobilized. More particularly, the monoclonal antibodies can be purified by chromatography on protein A and/or G, followed or not followed by ion-exchange chromatography aimed at eliminating the residual protein contaminants as well as the DNA and the lipopolysaccaride (LPS), in itself, followed or not followed by exclusion chromatography on Sepharose™ gel in order to eliminate the potential aggregates due to the presence of dimers or of other multimers. In one embodiment, the whole of these techniques can be used simultaneously or successively.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

Further techniques available in the art of antibody engineering have made it possible to isolate human and humanised antibodies. For example, human hybridomas can be made as described by Kontermann, R & Dubel, S, Antibody Engineering, Springer-Verlag New York, LLC; 2001, ISBN: 3540413545. Phage display, another established technique for generating antagonists has been described in detail in many publications, such as Kontermann & Dubel, supra and WO92/01047 (discussed further below), and US patents U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160 and U.S. Pat. No. 6,521,404.

Transgenic mice in which the mouse antibody genes are inactivated and functionally replaced with human antibody genes while leaving intact other components of the mouse immune system, can be used for isolating human antibodies Mendez, M. et al. (1997) Nature Genet, 15(2): 146-156. Humanised antibodies can be produced using techniques known in the art such as those disclosed in, for example, WO91/09967, U.S. Pat. No. 5,585,089, EP592106, U.S. Pat. No. 5,565,332 and WO93/17105. Further, WO2004/006955 describes methods for humanising antibodies, based on selecting variable region framework sequences from human antibody genes by comparing canonical CDR structure types for CDR sequences of the variable region of a non-human antibody to canonical CDR structure types for corresponding CDRs from a library of human antibody sequences, e.g. germline antibody gene segments. Human antibody variable regions having similar canonical CDR structure types to the non-human CDRs form a subset of member human antibody sequences from which to select human framework sequences. The subset members may be further ranked by amino acid similarity between the human and the non-human CDR sequences. In the method of WO2004/006955, top ranking human sequences are selected to provide the framework sequences for constructing a chimeric antibody that functionally replaces human CDR sequences with the non-human CDR counterparts using the selected subset member human frameworks, thereby providing a humanized antibody of high affinity and low immunogenicity without need for comparing framework sequences between the non-human and human antibodies. Chimeric antibodies made according to the method are also disclosed.

Synthetic antibody molecules may be created by expression from genes generated by means of oligonucleotides synthesized and assembled within suitable expression vectors, for example as described by Knappik et al. J. Mol. Biol. (2000) 296, 57-86 or Krebs et al. Journal of Immunological Methods 254 2001 67-84.

Note that regardless of how an antibody of interest is initially identified or made, any such antibody can be subsequently produced using recombinant techniques. For example, a nucleic acid sequence encoding the antibody may be expressed in a host cell. Such methods include expressing nucleic acid sequence encoding the heavy chain and light chain from separate vectors, as well as expressing the nucleic acid sequences from the same vector. These and other techniques using a variety of cell types are well known in the art.

Using these and other techniques known in the art, antibodies that specifically bind to any target can be made. Once made, antibodies can be tested to confirm that they bind to the desired target antigen and to select antibodies having desired properties. Such desired properties include, but are not limited to, selecting antibodies having the desired affinity and cross-reactivity profile. Given that large numbers of candidate antibodies can be made, one of skill in the art can readily screen a large number of candidate antibodies to select those antibodies suitable for the intended use. Moreover, the antibodies can be screened using functional assays to identify antibodies that bind the target and have a particular function, such as the ability to inhibit an activity of the target or the ability to bind to the target without inhibiting its activity. Thus, one can readily make antibodies that bind to a target and are suitable for an intended purpose.

The nucleic acid (e.g., the gene) encoding an antibody can be cloned into a vector that expresses all or part of the nucleic acid. For example, the nucleic acid can include a fragment of the gene encoding the antibody, such as a single chain antibody (scFv), a F(ab′)₂ fragment, a Fab fragment, or an Fd fragment.

Antibodies may also include modifications, e.g., modifications that alter Fc function, e.g., to decrease or remove interaction with an Fc receptor or with C1q, or both. For example, the human IgG4 constant region can have a Ser to Pro mutation at residue 228 to fix the hinge region.

In another example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. In another example, the Fc domain of IgG molecules is glycosylated at asparagine 297 in the CH2 domain. This asparagine is the site for modification with biantennary-type oligosaccharides. This glycosylation participates in effector functions mediated by Fcγ receptors and complement C1q (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, an antibody generated by a method described herein can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

For example, an antibody generated by a method described herein can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

Antibody-Mimic Molecules

Antibody-mimic molecules are antibody-like molecules comprising a protein scaffold or other non-antibody target binding region with a structure that facilitates binding with target molecules, e.g., polypeptides. When an antibody mimic comprises a scaffold, the scaffold structure of an antibody-mimic is reminiscent of antibodies, but antibody-mimics do not include the CDR and framework structure of immunoglobulins. Like antibodies, however, a pool of scaffold proteins having different amino acid sequence (but having the same basic scaffold structure) can be made and screened to identify the antibody-mimic molecule having the desired features (e.g., ability to bind a particular target; ability to bind a particular target with a certain affinity; ability to bind a particular target to produce a certain result, such as to inhibit activity of the target). In this way, antibody-mimics molecules that bind a target and that have a desired function can be readily made and tested in much the same way that antibodies can be. There are numerous examples of classes of antibody-mimic molecules; each of which is characterized by a unique scaffold structure. Any of these classes of antibody-mimic molecules may be used as the target-binding region of a protein entity of the disclosure. Exemplary classes are described below and include, but are not limited to, DARPin polypeptides and Anticalins® polypeptides.

In certain embodiments, an antibody-mimic moiety molecule can comprise binding site portions that are derived from a member of the immunoglobulin superfamily that is not an immunoglobulin (e.g., a T-cell receptor or a cell-adhesion protein such as CTLA-4, N-CAM, and telokin). Such molecules comprise a binding site portion which retains the conformation of an immunoglobulin fold and is capable of specifically binding to the target antigen or epitope. In some embodiments, antibody-mimic moiety molecules of the disclosure also comprise a binding site with a protein topology that is not based on the immunoglobulin fold (e.g., such as ankyrin repeat proteins) but which nonetheless are capable of specifically binding to a target antigen or epitope.

Antibody-mimic moiety molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in protein entity with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with the desired binding characteristics. For example, selection can be achieved by art-recognized methods such as phage display, yeast display, or ribosome display.

In one embodiment, an antibody-mimic molecule of the disclosure comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. The FnIII loops comprise regions that may be subjected to random mutation and directed evolutionary schemes of iterative rounds of target binding, selection, and further mutation in order to develop useful therapeutic tools. Fibronectin-based “addressable” therapeutic binding molecules (“FATBIM”) may be developed to specifically or preferentially bind the target antigen or epitope. Methods for making fibronectin binding polypeptides are described, for example, in WO 01/64942 and in U.S. Pat. Nos. 6,673,901, 6,703,199, 7,078,490, and 7,119,171, which are incorporated herein by reference.

In another embodiment, an antibody-mimic molecule of the disclosure comprises a binding site from an affibody. As used herein “Affibody®” molecules are derived from the immunoglobulin binding domains of staphylococcal Protein A (SPA) (see e.g., Nord et al., Nat. Biotechnol., 15: 772-777 (1997)). An Affibody® is an antibody mimic that has unique binding sites that bind specific targets. Affibody® molecules can be small (e.g., consisting of three alpha helices with 58 amino acids and having a molar mass of about 6 kDa), have an inert format (no Fc function), and have been successfully tested in humans as targeting moieties. Affibody® molecules have been shown to withstand high temperatures (90° C.) or acidic and alkaline conditions (pH 2.5 or pH 11, respectively). Affibody® binding sites employed in the disclosure may be synthesized by mutagenizing an SPA-related protein (e.g., Protein Z) derived from a domain of SPA (e.g., domain B) and selecting for mutant SPA-related polypeptides having binding affinity for a target antigen or epitope. Other methods for making affibody binding sites are described in U.S. Pat. Nos. 6,740,734 and 6,602,977 and in WO 00/63243, each of which is incorporated herein by reference. In certain embodiments, the disclosure provides a protein entity comprising a CPM associated with an Affibody, wherein the Affibody binds to an intraceullarly expressed target.

In another embodiment, an antibody-mimic molecule of the disclosure comprises a binding site from an anticalin. As used herein, “Anticalins®” are antibody functional mimetics derived from human lipocalins. Lipocalins are a family of naturally-occurring binding proteins that bind and transport small hydrophobic molecules such as steroids, bilins, retinoids, and lipids. The main structure of Anticalins® is similar to wild type lipocalins. The central element of this protein architecture is a beta-barrel structure of eight antiparallel strands, which supports four loops at its open end. These loops form the natural binding site of the lipocalins and can be reshaped in vitro by extensive amino acid replacement, thus creating novel binding specificities.

Anticalins® possess high affinity and specificity for their prescribed ligands as well as fast binding kinetics, so that their functional properties are similar to those of antibodies. Anticalins® however, have several advantages over antibodies, including smaller size, composition of a single polypeptide chain, and a simple set of four hypervariable loops that can be easily manipulated at the genetic level. Anticalins®, for example, are about eight times smaller than antibodies. Anticalins® have better tissue penetration than antibodies and are stable at temperatures up to 70° C., and also unlike antibodies, Anticalins® can be produced in bacterial cells (e.g., E. coli cells) in large amounts. Further, while antibodies and most other antibody mimetics can only be directed at macromolecules like proteins, Anticalins® are able to selectively bind to small molecules as well. Anticalins® are described in, e.g., U.S. Pat. No. 7,723,476. In certain embodiments, the disclosure provides a protein entity comprising a CPM associated with an Affibody, wherein the Affibody binds to an intraceullarly expressed target.

In another embodiment, an antibody-mimic molecule of the disclosure comprises a binding site from a cysteine-rich polypeptide. Cysteine-rich domains employed in the practice of the present disclosure typically do not form an alpha-helix, a beta-sheet, or a beta-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g. Sortilin-related receptor, LDL-receptor, VLDLR, LRP1, LRP2, and ApoER2). Methods for making A-domain proteins of a desired binding specificity are disclosed, for example, in WO 02/088171 and WO 04/044011, each of which is incorporated herein by reference.

In another embodiment, an antibody-mimic molecule of the disclosure comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include designed ankyrin repeat proteins (i.e., a DARPins) (see e.g., Binz et al., Nat. Biotechnol., 22: 575-582 (2004)) or leucine-rich repeat proteins (i.e., LRRPs) (see e.g., Pancer et al., Nature, 430: 174-180 (2004)).

As used here, “DARPins” are genetically engineered antibody mimetic proteins that typically exhibit highly specific and high-affinity target protein binding. DARPins were first derived from natural ankyrin proteins. In certain embodiments, DARPins comprise three, four or five repeat motifs of an ankyrin protein. In certain embodiments, a unit of an ankyrin repeat consists of 30-34 amino acid residues and functions to mediate protein-protein interactions. In ceratin embodiments, each ankyrin repeat exhibits a helix-turn-helix conformation, and strings of such tandem repeats are packed in a nearly linear array to form helix-turn-helix bundles connected by relatively flexible loops. In ceratin embodiments, the global structure of an ankyrin repeat protein is stabilized by intra- and inter-repeat hydrophobic and hydrogen bonding interactions. The repetitive and elongated nature of the ankyrin repeats provides the molecular bases for the unique characteristics of ankyrin repeat proteins in protein stability, folding and unfolding, and binding specificity. While not wishing to be bound by theory, it is believed that the ankyrin repeat proteins do not recognize specific sequences, and interacting residues are discontinuously dispersed into the whole molecules of both the ankyrin repeat protein and its target protein. In addition, the availability of thousands of ankyrin repeat sequences has made it feasible to use rational design to modify the specificity and stability of an ankyrin repeat domain for use as a DARPin to target any number of proteins. The molecular mass of a DARPin domain is typically about 14 or 18 kDa for four- or five-repeat DARPins, respectively. DARPins are described in, e.g., U.S. Pat. No. 7,417,130. All so far determined tertiary structures of ankyrin repeat units share a characteristic composed of a beta-hairpin followed by two antiparallel alpha-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Another example of a target-binding region suitable for use in the present disclosure is based on technology in which binding regions are engineered into the Fc domain of an antibody molecule. These antibody-like molecules are another example of target binding regions for use in the present disclosure. In certain embodiments, antibody mimics include all or a portion of an antibody like molecule, comprising the CH2 and CH3 domains of an immunoglulin, engineered with non-CDR loops of constant and/or variable domains, thereby mediating binding to an epitope via the non-CDR loops. Exemplary technology includes technology from F-Star, such as antigen binding Fc molecules (termed Fcab™) or full length antibody like molecules with dual functionality (MAb²™). Fcab™ (antigen binding Fc) are a “compressed” version of these antibody like molecules. These molecules include the CH2 and CH3 domains of the Fc portion of an antibody, naturally folded as a homodimer (50 kDa). Antigen binding sites are engineered into the CH3 domains, but the molecules lack traditional antibody variable regions.

Similar antibody like molecules are referred to as mAb²™ molecules. Full length IgG antibodies with additional binding domains (such as two) engineered into the CH3 domains. Depending on the type of additional binding sites engineered into the CH3 domains, these molecules may be bispecific or multispecific or otherwise facilitate tissue targeting.

This technology is described in, for example, WO08/003103, WO12/007167, and US application 20090298195, the disclosures of which are hereby incorporated by reference.

In other embodiments, an antibody-mimic molecule of the disclosure comprises binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Panni et al., J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, a binding domain derived from tetranectin in its monomeric or trimeric form, and other such domains known to those of ordinary skill in the art, as well as derivatives and/or variants thereof. Exemplary antibody-mimic moiety molecules, and methods of making the same, can also be found in Stemmer et al., “Protein scaffolds and uses thereof”, U.S. Patent Publication No. 20060234299 (Oct. 19, 2006) and Hey, et al., Artificial, Non-Antibody Binding Proteins for Pharmaceutical and Industrial Applications, TRENDS in Biotechnology, vol. 23, No. 10, Table 2 and pp. 514-522 (October 2005).

In one embodiment, an antibody-mimic molecule comprises a Kunitz domain. “Kunitz domains” as used herein, are conserved protein domains that inhibit certain proteases, e.g., serine proteases. Kunitz domains are relatively small, typically being about 50 to 60 amino acids long and having a molecular weight of about 6 kDa. Kunitz domains typically carry a basic charge and are characterized by the placement of two, four, six or eight or more that form disulfide linkages that contribute to the compact and stable nature of the folded peptide. For example, many Kunitz domains have six conserved cysteine residues that form three disulfide linkages. The disulfide-rich α/β fold of a Kunitz domain can include two, three (typically), or four or more disulfide bonds.

Kunitz domains have a pear-shaped structure that is stabilized the, e.g., three disulfide bonds, and that contains a reactive site region featuring the principal determinant P1 residue in a rigid confirmation. These inhibitors competitively prevent access of a target protein (e.g., a serine protease) for its physiologically relevant macromolecular substrate through insertion of the P1 residue into the active site cleft. The P1 residue in the proteinase-inhibitory loop provides the primary specificity determinant and dictates much of the inhibitory activity that particular Kunitz protein has toward a targeted proteinase. Typically, the N-terminal side of the reactive site (P) is energetically more important that the P′ C-terminal side. In most cases, lysine or arginine occupy the P1 position to inhibit proteinases that cleave adjacent to those residues in the protein substrate. Other residues, particularly in the inhibitor loop region, contribute to the strength of binding. Generally, about 10-12 amino acid residues in the target protein and 20-25 residues in the proteinase are in direct contact in the formation of a stable proteinase-inhibitor protein entity and provide a buried area of about 600 to 900 A. By modifying the residues in the P site and surrounding residues Kunitz domains can be designed to target and inhibit a protein of choice. Kunitz domains are described in, e.g., U.S. Pat. No. 6,057,287.

In another embodiment, an antibody-mimic molecule of the disclosure is an Affilin®. As used herein “Affilin®” molecules are small antibody-mimic proteins which are designed for specific affinities towards proteins and small compounds. New Affilin® molecules can be very quickly selected from two libraries, each of which is based on a different human derived scaffold protein. Affilin® molecules do not show any structural homology to immunoglobulin proteins. There are two commonly-used Affilin® scaffolds, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

In another embodiment, an antibody-mimic moiety molecule of the disclosure is an Avimer. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. In certain embodiments, Avimers consist of two or more peptide sequences of 30 to 35 amino acids each, connected by spacer region peptides. The individual sequences are derived from A domains of various membrane receptors and have a rigid structure, stabilised by disulfide bonds and calcium. Each A domain can bind to a certain epitope of the target protein. The combination of domains binding to different epitopes of the same protein increases affinity to this protein, an effect known as avidity (hence the name). Other potential advantages include simple and efficient production of multitarget-specific molecules in Escherichia coli, improved thermostability and resistance to proteases. Avimers with sub-nanomolar affinities have been obtained against a variety of targets. Alternatively, the domains can be directed against epitopes on different target proteins. This approach is similar to the one taken in the development of bispecific monoclonal antibodies. In a study, the plasma half-life of an anti-interleukin 6 avimer could be increased by extending it with an anti-immunoglobulin G domain. Additional information regarding Avimers can be found in U.S. patent application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

The foregoing provides numerous examples of classes of antibody-mimics. In certain embodiments, the disclosure provides protein entities in which the target-binding region is an antibody-mimic that binds to a cell surface target at the cell surface, such as any of the foregoing classes of antibody-mimics Any of these antibody-mimics may be connected with (e.g., combined or fused with) a CPM or a portion comprising a CPM, including any of the sub-categories or specific examples of CPM. Regardless of the particular category of target binding region selected, the target binding region binds a cell surface target. In the context of a protein entity, the target binding region binds the cell surface target at the cell surface, and thus localizes the protein entity to cells of interest. In that way, the target binding region (cell surface target binding region) is able to effect penetration.

Adhesin Molecules

Adhesin molecules comprise a ligand, a receptor, or portions thereof (an “adhesin”). In certain embodiments, the disclosure provides protein entities in which the target-binding region is an adhesin molecule.

In certain embodiments, adhesins are chimeric molecules which combine the binding domain of a protein such as a cell-surface receptor or a ligand with a portion of an immunoglobulin molecule, e.g., the effector domain or constant domain; at least one domain of an Ig constant region; one or more domain selected from C_{H}1, C_{H}2, C_{H}3, or C_{H}4. Adhesins can possess many of the valuable chemical and biological properties of antibodies.

A binding domain of a ligand refers to any native cell-surface receptor or any region or derivative thereof retaining at least a qualitative ligand binding ability, and preferably the biological activity of a corresponding native receptor. In a specific embodiment, the receptor is from a cell-surface polypeptide having an extracellular domain which is homologous to a member of the immunoglobulin supergenefamily. Other typical receptors, are not members of the immunoglobulin supergenefamily but are nonetheless specifically covered by this definition, are receptors for cytokines, and in particular receptors with tyrosine kinase activity (receptor tyrosine kinases), members of the hematopoietin and nerve growth factor receptor superfamilies, and cell adhesion molecules, e.g. (E-, L- and P-) selectins.

A binding domain of a receptor is used to designate any native ligand for a receptor, including cell adhesion molecules, or any region or derivative of such native ligand retaining at least a qualitative receptor binding ability, and preferably the biological activity of a corresponding native ligand.

Adhesins can be constructed from a human protein sequence with a desired specificity linked to an appropriate human immunoglobulin hinge and constant domain (Fc) sequence and thus, the binding specificity of interest can be achieved using entirely human components. Such adhesins are minimally immunogenic to the patient, and are safe for chronic or repeated use.

Adhesins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin or homing receptor (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); NP receptors (Bennett et al., J. Biol. Chem. 266:23060-23067 (1991)); inteferon .gamma. receptor (Kurschner et al., J. Biol. Chem. 267:9354-9360 (1992)); 4-1BB (Chalupny et al., PNAS (USA) 89:10360-10364 (1992)) and IgE receptor .alpha. (Ridgway and Gorman, J. Cell. Biol. Vol. 115, Abstract No. 1448 (1991)).

Preparation of Adhesin Molecules

Chimeras constructed from an adhesin binding domain sequence, optionally linked to an appropriate immunoglobulin constant domain sequence (adhesins) are known in the art.

The simplest and most straightforward adhesin design combines the binding domain(s) of the adhesin (e.g., the extracellular domain (ECD) of a receptor) with the hinge and Fc regions of an immunoglobulin heavy chain. Ordinarily, when preparing the adhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion, or binding characteristics of the Ia.

In a specific embodiment, the adhesin sequence is fused to the N-terminus of the Fc domain of immunoglobulin G1 (IgG1). It is possible to fuse the entire heavy chain constant region to the adhesin sequence. In another embodiment, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In another specific embodiment, the adhesin amino acid sequence is fused to (a) the hinge region and CH2 and CH3 or (b) the CH1, hinge, CH2 and CH3 domains, of an IgG1, IgG2, or IgG3 heavy chain. The precise site at which the fusion is made is not critical, and the optimal site can be determined by routine experimentation.

The foregoing provide examples of categories of molecule that are suitable for use as a target binding region in the protein entities of the disclosure. The particular architecture can be chosen based on numerous factors, such as prior availability, desired affinity and KD, ease of manufacture, and the like. Target binding regions are connected to a CPM to provide a protein entity of the disclosure. Suitable connection, including by making a fusion protein joining at least one unit of the target binding moiety to at least one unit of the CPM, directly or via a primary SR, schemes are chosen depending on the target binding region and CPM.

The disclosure contemplates that any of the categories of target binding regions described herein, including target binding regions having any one or combination of structural and functional properties described herein, may be combined to produce a protein entity with any of the CPM or categories of CPMs described herein, including CPMs having any one or combination of structural and functional properties described herein.

Regardless of the particular category of target binding region selected, the target binding region binds a cell surface target. In the context of a protein entity, the target binding region binds the cell surface target at the cell surface, and thus contributes to localizing the protein entity into specific cells of interest. This is amongst the mechanisms by which the target binding region effects penetration by localizing the protein entity.

Dissociation Constants and Avidity

The term “K_{D}” or “dissociation constant”, as used herein, is intended to refer to the “equilibrium dissociation constant”, and refers to the value obtained in a titration measurement at equilibrium, or by dividing the dissociation rate constant (k_{off}) by the association rate constant (kₒₙ). The association rate constant, the dissociation rate constant and the equilibrium dissociation constant are used to represent the binding affinity of a target binding region (e.g., an antibody fragment, such as an scFv) to a cell surface target (e.g., its antigen). Methods for determining association and dissociation rate constants are known in the art. For example, fluorescence-based techniques can offer high sensitivity and the ability to examine samples in physiological buffers at equilibrium. Other experimental approaches and instruments such as a BIAcore™ (biomolecular interaction analysis) assay can be used (e.g., instrument available from BIAcore International AB, a GE Healthcare company, Uppsala, Sweden). Additionally, a KinExA™ (Kinetic Exclusion Assay) assay, available from Sapidyne Instruments (Boise, Id.) can also be used.

The term “avidity” refers to the combined strength of multiple bond interactions, such as the compound affinity of multiple antibody/antigen interactions. Antibody avidity may be measured using methods known in the art which assess degree of binding of antibody to antigen. These methods include competition assays and non-competition assays.

In certain embodiments, the target binding region that can be used in the protein entity of the present disclosure binds the cell surface target with a dissociation constant (K_{D}) of greater than 0.01 nM or with an avidity of greater than 0.001 nM. In certain embodiments, the target-binding region binds the cell surface target with a K_{D} or avidity at least greater than 0.02 nM, 0.03 nM, 0.04 nM, 0.05 nM, 0.1 nM, 0.2 nM, 0.3 nM, 0.4 nM, 0.5 nM, 0.6 nM, 0.7 nM, 0.8 nM, 0.9 nM, or 1 nM. In certain embodiments, the target-binding region binds the cell surface target with a K_{D} or an avidity of at least greater than 2 nM, 3 nM, 4 nM, 5 nM, 10 nM, 100 nM, 200 nM, 300 nM, 400 nM, 500 nM, 600 nM, 700 nM, 800 nM, or 900 nM. In certain embodiments, the target-binding region binds the cell surface target with a K_{D} or avidity at least greater than 0.002 nM, 0.003 nM, 0.004 nM, 0.005 nM, 0.01 nM, 0.02 nM, 0.03 nM, 0.04 nM, 0.05 nM, 0.06 nM, 0.07 nM, 0.08 nM, 0.09 nM, or 0.1 nM. In certain embodiments, the target-binding region binds the cell surface target with a K_{D} or an avidity of at least greater than 2 nM, 3 nM, 4 nM, 5 nM, 10 nM, 100 nM, 200 nM, 300 nM, 400 nM, 500 nM, 600 nM, 700 nM, 800 nM, or 900 nM.

In certain embodiments, the target-binding region binds the cell surface target with a K_{D} or avidity of about 0.01 nM, 0.02 nM, 0.03 nM, 0.04 nM, 0.05 nM, 0.1 nM, 0.2 nM, 0.3 nM, 0.4 nM, 0.5 nM, 0.6 nM, 0.7 nM, 0.8 nM, 0.9 nM, or 1 nM. In certain embodiments, the target-binding region binds the cell surface target with a K_{D} or an avidity of about 2 nM, 3 nM, 4 nM, 5 nM, 10 nM, 100 nM, 200 nM, 300 nM, 400 nM, 500 nM, 600 nM, 700 nM, 800 nM, or 900 nM. In certain embodiments, the target-binding region binds the cell surface target with a K_{D} or avidity of about 0.002 nM, 0.003 nM, 0.004 nM, 0.005 nM, 0.01 nM, 0.02 nM, 0.03 nM, 0.04 nM, 0.05 nM, 0.06 nM, 0.07 nM, 0.08 nM, 0.09 nM, or 0.1 nM. In certain embodiments, the target-binding region binds the cell surface target with a K_{D} or an avidity of at least greater than 2 nM, 3 nM, 4 nM, 5 nM, 10 nM, 100 nM, 200 nM, 300 nM, 400 nM, 500 nM, 600 nM, 700 nM, 800 nM, or 900 nM.

In certain embodiments, the target-binding region binds the cell surface target with a dissociation constant (K_{D}) of less than 1 μM or with an avidity of less than 1 μM. In certain embodiments, the target-binding region binds the cell surface target with a K_{D} or an avidity of no more than (e.g., less than) 100 nM, 200 nM, 300 nM, 400 nM, 500 nM, 600 nM, 700 nM, 800 nM, 900 nM, or 1 μM. In certain embodiments, the target-binding region binds the cell surface target with a K_{D} or an avidity of no more than (e.g., less than) 1 nM, 2 nM, 3 nM, 4 nM, 5 nM, 10 nM, 20 nM, 30 nM, 40 nM, 50 nM, 60 nM, 70 nM, 80 nM, or 90 nM.

In certain embodiments, the target-binding region binds the cell surface target with a dissociation constant (K_{D}) of less than 1 μM or with an avidity of less than 1 μM. In certain embodiments, the target-binding region binds the cell surface target with a K_{D} or an avidity of about 100 nM, 200 nM, 300 nM, 400 nM, 500 nM, 600 nM, 700 nM, 800 nM, 900 nM, or 1 μM. In certain embodiments, the target-binding region binds the cell surface target with a K_{D} or an avidity of about 1 nM, 2 nM, 3 nM, 4 nM, 5 nM, 10 nM, 20 nM, 30 nM, 40 nM, 50 nM, 60 nM, 70 nM, 80 nM, or 90 nM.

In certain embodiments, the target-binding region binds the cell surface target with a dissociation constant (K_{D}) or with an avidity greater than 0.01 nM and less than 1 μM, or between 0.1 nM to 1 μM, or between 0.1 nM to 100 nM (see Tables 1 and 2). The disclosure contemplates target binding regions that bind (e.g., specifically bind) a cell surface target with a dissociation constant (K_{D}) or with an avidity greater within any range bounded by any of the values set forth above.

TABLE 1
Exemplary K_{D} Ranges of Target-binding regions

| | | Lower range | | | | | |
|---|---|---|---|---|---|---|---|
| Upper range | | 0.01 nM | 0.1 nM | 1 nM | 10 nM | 50 nM | 100 nM |
| 0.1 | nM | + | | | | | |
| 1 | nM | + | + | | | | |
| 10 | nM | + | + | + | | | |
| 50 | nM | + | + | + | + | | |
| 100 | nM | + | + | + | + | + | |
| 1 | μM | + | + | + | + | + | + |

TABLE 2
Exemplary Avidity Ranges of Target-binding regions

| | | Lower range | | | | | |
|---|---|---|---|---|---|---|---|
| Upper range | | 0.001 nM | 0.01 nM | 0.1 nM | 1 nM | 10 nM | 100 nM |
| 0.01 | nM | + | | | | | |
| 0.1 | nM | + | + | | | | |
| 1 | nM | + | + | + | | | |
| 10 | nM | + | + | + | + | | |
| 100 | nM | + | + | + | + | + | |
| 1 | μM | + | + | + | + | + | + |

The disclosure contemplates that the target binding region may be selected based on its affinity for a particular cell surface target. The affinity and binding kinetics of the target binding region are chosen to provide, in combination with the selected CPM to which it will be appended, to provide balance between the target mediated binding function of the target binding region and the internalization function of the CPM. The balance may vary for different target binding region/CPM pairs, and may also vary depending on the level of expression of the target on the cell surface of the cells for which delivery is desired. In the context of a protein entity, the balance is such that the target binding region binds the cell surface target at the cell surface and contributes to localization of the protein entity at cells of interest. In other words, enhanced cell penetration is influenced by both the activity of the target binding region at the cell surface and that of the CPM.

In certain embodiments, the target binding region does not specifically bind heparin sulfate.

It should be understood that the target binding region helps target the protein entity to a cell or tissue expressing its antigen at the cells surface (e.g., the cell surface target). This targeting prevents ubiquitous cell penetration, and helps enrich penetration to the desired cells and tissues. It is understood that targeting is not meant to imply that the protein entity is delivered exclusively to cells expressing the cell surface target. However, the protein entity is delivered non-ubiquitously, as a function of cell surface target expression, and delivery is enriched, significantly, to cells expressing the cell surface target. In the context of a protein entity, the target binding region binds the cell surface target at the cell surface, and thus contributes to localization of the protein entity to the surface of cells of interest. This is an example of how the target binding region effects cell penetration by localizing the protein entity at the cell surface of cells of interest.

The disclosure contemplates all combinations of any of the foregoing aspects and embodiments with each other, as well as combinations with any of the embodiments set forth in the detailed description and examples. Any of the structural and/or functional features of the target binding region may be combined with each other, as well as with any one or more of the structural and/or functional features of other components of the disclosure.

### (iii) Cell Surface Target and Targeted Cells

The term “cell surface target,” as used herein, refers to a molecule that is expressed on the cell surface. By “expressed on the cell surface” it is meant that (i) at least one region of the target is associated, directly or indirectly, with the cell membrane, and (ii) an extracellular domain or surface-exposed bindable segments of the target render it accessible for association with the target binding region. The term “targeted cell(s)” refers to cells that express a cell surface target of interest. The protein entity of the present disclosure binds a cell surface target at the cell surface as a function of the target-binding region and internalizes into the cells as a function of the CPM. In the context of a protein entity, the target binding region binds the cell surface target at the cell surface, and thus contributes to localization of the protein entity to cells of interest. The protein entity, either being a therapeutic agent itself, or conjugated to a cargo region, after internalization into the targeted cells, may regulate a biological activity of the cells and thus achieve the effect of treating disease or curing a protein deficiency, or may provide useful tools for in vitro studies, or imaging or diagnostic reagents.

The protein entities of the present disclosure promote targeted delivery of to specific cell types, as a function of the target binding region. For example, the protein entity comprising a target-binding region (such as an anti-Her2 antibody or anti-Her2 scFv) and a CPM (such as a CPM of T-cell surface antigen CD2) can promote targeted delivery and enhanced penetration of the target-binding region, which is a therapeutic agent by itself, to cells expressing Her2. By way of further example, the protein entity of the present disclosure is further conjugated to a cargo (e.g., a cytotoxic agent) and the protein entity promotes the targeted delivery and internalization of the cargo into targeted cells. Without being bound by theory, the presence of the target-binding region increases the targeting specificity of the protein entity and the presence of the CPM increases the penetration capacity of the protein entity. Thus, the protein entity of the present disclosure can bind specifically to a cell surface target of interest on a targeted cell and further be internalized into the targeted cells. In the context of a protein entity, the target binding region binds the cell surface target at the cell surface, and thus contributes to localization of the protein entity to cells of interest.

Examples of targeted cells include, without limitation, cancer cells, cells of the immune system (e.g., T-cells, B-cells, lymphocytes etc.), or cells that express proteins having extracellular domains overexpressed on the surface. In certain embodiments, the targeted cells express growth factor receptors (e.g., Her2 or EGFR, TNFR, FGFR), G-protein couple receptors (GPCRs), ion channel proteins, lectin/sugar binding proteins (e.g., CD22), GPI-anchored proteins (e.g., CD52), integrins or the subunits thereof (e.g., CD11a or alpha 4 integrin), cell type specific receptors (e.g., B cell receptors such as CD20 or a T cell receptor), or proteins having an extracellular domain overexpressed on the surface of a desired cell type. The protein entities of the present disclosure may target these cells by specifically binding a cell surface target expressed on the targeted cell surface as a function of at least its target binding region and further effect the internalization as a function of its CPM.

In certain embodiments, the cell surface target is a growth factor receptor, G-protein couple receptor, an ion channel protein, a lectin/sugar binding protein, a GPI-anchored protein (e.g., CD52), an integrin or subunit thereof, a cell type specific receptor, such as a B- or T-cell specific receptor, or a protein having an extracellular domain overexpressed on the surface of a desired cell type

Examples of cell surface targets include CD30, Her2, ectonucleotide pyrophosphatase/phosphodiesterase 3 (ENPP3), CD22, EGFR, TNFR, FGFR, CD20, CD52, CD11a and alpha4-integrin. In certain embodiments, the target binding region that binds to cells expressing CD30 includes brentuximab and antibody fragments or variants thereof (such as a scFv). The target binding region that binds to cells expressing Her2 includes trastuzumab and antibody fragments or variants thereof (such as a scFv-C6.5; see examples). The target binding region that binds to cells expressing CD22 includes inotuzumab and antibody fragments or variants thereof (such as a scFv). The target binding region that binds to cells expressing CD20 includes rituximab and antibody fragments or variants thereof (such as a scFv). The target binding region that binds to cells expressing CD52 includes alemtuzumab and antibody fragments or variants thereof (such as a scFv). The target binding region that binds to cells expressing CD11a includes efalizumab and antibody fragments or variants thereof (such as a scFv). The target binding region that binds to cells expressing alpha4-integrin includes natalizumab and antibody fragments or variants thereof (such as a scFv).

Note that the antibodies noted above are exemplary of target binding regions that bind a cell surface target. Such antibodies or antigen binding fragments thereof may be used in a protein entity of the disclosure, such as described in the examples using an scFv based on one of these antibodies.

The disclosure contemplates all combinations of any of the foregoing aspects and embodiments with each other, as well as combinations with any of the embodiments set forth in the detailed description and examples.

### (iv) Charged Protein Moiety

The term “charged protein moiety,” as used herein, refers to a positively charged molecule that is capable of promoting penetration across cellular membranes and into cells of itself, and is also capable of promoting or enhancing penetration of the protein entities into cells. In certain embodiments, the charged protein moiety (CPM) comprise at least one polypeptide capable of promoting penetration into a cell and having, at least, the following characteristics: net positive charge, tertiary structure (e.g., the CPM is a globular protein), mass of at least 4 kDa, a net theoretical charge of less than +20, and presence of surface positive charge such that the polypeptide is capable of promoting penetration into a cell. Additionally or alternatively, in certain embodiments, the charged protein moiety (CPM) comprise at least one polypeptide capable of promoting penetration into a cell and having, at least, the following characteristics: net positive charge, tertiary structure (e.g., the CPM is a globular protein), mass of at least 4 kDa, charge per molecular weight ratio of less than 0.75, and presence of surface positive charge such that the polypeptide is capable of promoting penetration into a cell. Note that when the CPM comprises two polypeptide chains, these characteristics are the features of each chain. In certain embodiments, a CPM is a charge-engineered immunoglobulin region (such as a charge-engineered C_{H}3 domain). In certain embodiments, the CPM is a variant of a naturally occurring protein, in which the variant has one or more amino acid substitutions, additions, or deletions to increase net positive charge, surface charge, or charge to molecular weight ratio relative to that of the of the starting protein (e.g., the naturally occurring protein).

In certain embodiments, the charged protein moiety (CPM) comprise at least one polypeptide capable of promoting penetration into a cell and having, at least, the following characteristics: net positive charge, tertiary structure (e.g., the CPM is a globular protein), mass of at least 4 kDa, a net theoretical charge of at least +3, +4, +5, or +6, charge per molecular weight ratio of less than 0.75, and presence of surface positive charge such that the polypeptide is capable of promoting penetration into a cell. Note that when the CPM comprises two polypeptide chains, these characteristics are the features of each chain. In certain embodiments, a CPM is a charge-engineered immunoglobulin region (such as a charge-engineered C_{H}3 domain). In certain embodiments, the CPM is a variant of a naturally occurring protein, in which the variant has one or more amino acid substitutions, additions, or deletions to increase net positive charge, surface charge, or charge to molecular weight ratio relative to that of the of the starting protein (e.g., the naturally occurring protein). In certain embodiments, the CPM does not comprise a C_{H}3 domain. In certain embodiments, the CPM

The CPM can bind to proteoglycans and promote proteoglycan-mediated internalization into cells expressing the cell surface target. A CPM may be a human polypeptide, including a full length, naturally occurring human polypeptide or a variant of a full length, naturally occurring human polypeptide having one or more amino acid additions, deletions, or substitutions. Moreover, such human polypeptides include domains of full length naturally occurring human polypeptides or a variant of such a domain having one or more amino acid additions, deletions, or substitutions. For the avoidance of doubt, the term “human polypeptide” includes domains (e.g., structural and functional fragments) unless otherwise specified. Further, CPMs include human or non-human proteins engineered to have one or more regions of surface positive charge and a net theoretic positive charge. The present disclosure provides numerous examples of CPMs, as well as numerous examples of sub-categories of CPMs. The disclosure contemplates that any of the sub-categories of CPMs, as well as any of the specific polypeptides described herein may be provided as part of a protein entity comprising a target-binding region. Moreover, any such protein entities may be used to deliver a cargo into a cell.

In the present context, a “variant of a human polypeptide” is a polypeptide that differs from a naturally occurring (full length or domain) polypeptide, such as a human polypeptide, by one or more amino acid substitutions, additions or deletions. In certain embodiments, these changes in amino acid sequence may be to increase the overall net charge of the polypeptide and/or to increase the surface charge of the polypeptide (e.g., to supercharge a polypeptide). Alternatively, changes in amino acid sequence may be for other purposes, such as to provide a suitable site for pegylation or to facilitate production. Regardless of the specific changes made, the variant of the human polypeptide will be sufficiently similar based on sequence and/or structure to its naturally occurring human polypeptide such that the variant is more closely related to the naturally occurring human protein than it is to a protein from a non-human organism. In certain embodiments, the amino acid sequence of the variant is at least 80%, 85%, 90%, 95%, 97%, 98%, or 99% identical to a naturally occurring protein. In certain embodiments, the variant of the naturally occurring polypeptide is a CPM having cell penetrating activity, surface positive charge, and a net theoretical charge of greater than +2 and less than +20, but the naturally occurring polypeptide from which the variant is derived does not have cell penetrating activity. In certain embodiments, the variant does not result in further charge-engineering of the polypeptide. For example, the variant results in a change in amino acid sequence but not a change in the net charge, surface charge and/or charge/molecular weight ratio of the polypeptide.

In certain embodiments, the CPM is a polypeptide, such as a human polypeptide that is a domain of a naturally occurring human polypeptide. In addition to having surface positive charge and the ability to penetrate cells, the domain of a naturally occurring human polypeptide has a mass of at least 4 kDa. Additionally or alternatively, in certain embodiments, such a domain has an overall net positive charge greater than that of the corresponding, full length, naturally occurring human protein.

In certain embodiments, a CPM has a mass of at least 4, 5, 6, 10, 20, 50, 65, 75, 100, 200 kDa or 250 kDa. For example, a CPM may have a mass of about 4, 5, 6, 7, 8, 9, 10, 11, 12, 13, 14, 15, 16, 17, 18, 19, 20, 21, 22, 23, 24, 25, 26, 27 or 28 kDa. By way of another example, a CPM may have a mass of about 25-85 kDa, 40-80 kDa, 50-75, kDa, 65-75 kDa, 4-30 kDa, about 5-25 kDa, about 4-20 kDa, about 5-18 kDa, about 5-15 kDa, about 4-12 kDa, about 5-10 kDa, and the like. In still other embodiments, the molecular weight of a CPM (e.g., a naturally occurring or modified CPM protein) ranges from approximately 5 kDa to approximately 250 kDa, such as 10 to 250 kDa, 50 to 250 kDa, or 50 to 100 kDa. For example, in certain embodiments, the molecular weight of the CPM ranges from approximately 4 kDa to approximately 100 kDa. In certain embodiments, the molecular weight of the CPM ranges from approximately 10 kDa to approximately 45 kDa. In certain embodiments, the molecular weight of the CPM ranges from approximately 5 kDa to approximately 50 kDa. In certain embodiments, the molecular weight of the CPM ranges from approximately 5 kDa to approximately 27 kDa. In certain embodiments, the molecular weight of the CPM ranges from approximately 10 kDa to approximately 60 kDa. In certain embodiments, the molecular weight of the CPM is about 5 kD, about 7.5 kDa, about 10 kDa, about 12.5 kDa, about 15 kDa, about 17.5 kDa, about 20 kDa, about 22.5 kDa, about 25 kDa, about 27.5 kDa, about 30 kDa, about 32.5 kDa, or about 35 kDa. It should be understood that the mass of the CPM, including the minimal mass of 4 kDa, refers to monomer mass. However, in certain embodiments, a CPM for use as part of a protein entity is a dimer, trimer, tetramer, or a higher order multimer. In certain embodiments, where the CPM is a fragment of another protein, the protein entity does not include additional amino acid sequence contiguous with the CPM from that same protein. In certain embodiments, where the CPM is a fragment of another protein, the protein entity does not include additional amino acid sequence from the same protein.

In certain embodiments, a CPM for use in the present disclosure is selected to minimize the number of disulfide bonds. In other words, the CPM may have not more than 2 or 3 or 4 disulfide bonds (e.g., the polypeptide has 0, 1, 2, 3 or 4 disulfide bonds). A CPM for use in the present disclosure may also be selected to minimize the number of cysteines. In other words, the CPM may have not more than 2 cysteines, or not more than 4 cysteines, not more than 6 cysteines or not more than 8 cysteines (e.g., 0, 1, 2, 3, 4, 5, 6, 7, 8 cysteines). A CPM for use in the present disclosure may also be selected to minimize glycosylation sites. In other words, the polypeptide may have not more than 1 or 2 or 3 glycosylation sites (e.g., N-linked or O-linked glycosylation; 0, 1, 2 or 3 sites). In certain embodiments, amino acid substitutions can be introduced to eliminate one or more N- or O-linked glycosylation sites.

The CPM of the present disclosure has a net theoretic positive charge. In some embodiments, the CPM has a net theoretical charge of from about +2 to about +15. In some embodiments, the CPM has a net theoretical charge of from about +3 to about +12. In some embodiments, the CPM has a net theoretical charge of from about +5 to about +15, or about +5 to about +15, or about +6 to about +12. For example, the CPM has a net theoretical charge of about +2, +3, +4, +5, +6, +7, +8, +9, +10, +11, +12, +13, +14, +15, +16, +17, +18, +19. In certain embodiments, the CPM has a net theoretical charge of about +20 or +21. In some embodiments, the CPM has a charge per molecular weight ratio of less than 0.75. In some embodiments, the CPM has a charge per molecular weight ratio of from about 0.2 to about 0.6. In some embodiments, the CPM has a charge per molecular weight ratio of greater than 0 to about 0.25. For example, the CPM has a charge per molecular weight ratio of about 0.1, 0.2, 0.25, 0.3, 0.35, 0.4, 0.45, 0.5, 0.55, 0.6, 0.65, or 0.7.

As defined above, a CPM has surface positive charge and, preferably, a net positive charge. The CPM also has an overall net positive charge, which may be dispersed over a large part of the surface or quite spatially localized at one or more sites on the CPM surface, under physiological conditions. Note that when the CPM is a domain of a naturally occurring polypeptide, the overall net positive charge is that of the domain. In some embodiments, the CPM has a net theoretical charge of from about +2 to about +15. In some embodiments, the CPM has a net theoretical charge of from about +3 to about +12. For example, the CPM has a net theoretical charge of about +2, +3, +4, +5, +6, +7, +8, +9, +10, +11, +12, +13, +14, or +15. Note that a CPM may be a polypeptide that has been modified, such as to increase surface charge and/or overall net positive charge as compared to the unmodified protein, and the modified polypeptide may have increased stability and/or increased cell penetrating ability in comparison to the unmodified polypeptide. In some cases, the modified polypeptide may have cell penetrating ability where the unmodified polypeptide did not.

Theoretical net charge serves as a convenient short hand. In certain embodiments, the theoretical net charge on the CPM (e.g., the naturally occurring CPM or the modified CPM) is at least +2, +3, +4, +5, +6, +7, +8, +9, +10, +11, +12, +13, +14, or +15. In certain embodiments, the theoretical net charge is from +6 to +15, +6 to +18, +9 to +20, +9 to +18, or +9 to +15. For example, the theoretical net charge on the naturally occurring CPM can be, e.g., at least +1, at least +2, at least +3, at least +4, at least +5, at least +6, at least +7, at least +8, at least +9, at least +10, at least +11, at least +12, at least +13, at least +14, at least +15, or about +1 to +5, +1 to +10, +5 to +10, +5 to +15, and the like. Note that a CPM may be a polypeptide that has been modified, such as to increase surface charge and/or overall net positive charge as compared to the unmodified protein (e.g., the starting protein), and the modified polypeptide may have increased stability and/or increased cell penetrating ability in comparison to the unmodified polypeptide. In some cases, the modified polypeptide may have cell penetrating ability where the unmodified polypeptide did not.

In certain embodiments, the CPM has a charge:molecular weight ratio (e.g., also referred to as charge/MW or charge/molecular weight) of less than 0.75. This ratio is the ratio of the theoretical net charge of the CPM to its molecular weight in kilodaltons. In certain embodiments, the CPM is a domain of a naturally occurring human polypeptide where the domain has a charge/molecular weight ratio of less than 0.75.

For example, in certain embodiments, the CPM has a charge:molecular weight ratio of less than 0.75. In certain embodiments, the CPM has a charge:molecular weight ratio of less than 0.6. In certain embodiments, the CPM has a charge:molecular weight ratio of less than 0.5. In certain embodiments, the CPM has a charge:molecular weight ratio of less than 0.4. In certain embodiments, the CPM has a charge:molecular weight ratio of less than 0.3. In certain embodiments, the CPM has a charge:molecular weight ratio of less than 0.25. In certain embodiments, the CPM has a charge:molecular weight ratio of greater than 0. In certain embodiments, the CPM has a charge per molecular weight ratio of 0.2-0.5 or 0.2-0.6. In certain embodiments, the CPM has a charge per molecular weight ratio of 0.2-0.5 or 0.2-0.6 and a theoretical net charge of about +6 to +15, about +9 to +18, about +9 to +15, or about +9 to +20.

In certain embodiments, the CPM has a pI of about 9-10.5, or about 9-10.2, or about 9.6-10.1.

In certain embodiments, the CPM comprises a naturally occurring protein, such as a human protein. In certain embodiments, the CPM comprises a variant of a naturally occurring human protein (e.g., a charge engineered variant). In certain embodiments, the CPM is a domain of a naturally occurring protein.

In certain embodiments, the CPM is a variant having at least two amino acid substitutions, additions, or deletions relative to a starting protein (e.g., a naturally occurring protein) and wherein the CPM has a greater net theoretical charge than the starting protein by at least +2. In certain embodiments, the CPM is a variant having at least three, at least four, at least five, at least six, at least seven, at least 8, at least 9, or at least 10 amino acid substitutions relative to a starting protein. In certain embodiments, CPM is a variant having from 2-10 amino acid substitutions relative to a starting protein. In certain embodiments, the CPM has a greater net theoretical charge than the starting protein by at least +3, at least +4, at least +5, at least +6, at least +7, at least +8, at least +9, at least +10, at least +12, at least +14, at least +16, or at least +18. In certain embodiments, the CPM has a greater net theoretical charge than the starting protein by from +3 to +15.

In certain embodiments, the CPM comprises an immunoglobulin (Ig) C_{H}3 domain which has been altered to increase its surface positive charge and/or net positive charge to promote internalization into cells. In certain embodiments, the CPM comprises a pair of human C_{H}3 domains, of which the amino acid sequence of at least one domain has been altered to increase surface positive charge and/or net positive charge to promote internalization into cells. Note that when a C_{H}3 domain of an Ig is present as a pair of polypeptides (e.g., a pair of C_{H}3 domains) one or both domains may be charge modified and any charge modification is independently selected. In certain embodiments, altering of the amino acid sequence comprises introducing at least 3, at least 4, at least 5, at least 6, at least 7, or at least 8 amino acid substitutions, independently, into one or, if present, both C_{H}3 domains to increase surface positive charge, net positive charge, and/or charge per molecular weight ratio of the CPM. In certain embodiments, C_{H}3 domains are from human IgG and their charge engineering does not interfere with normal neonatal Fc receptor binding and cellular recycling. In certain embodiments, the C_{H}3 domains are from human IgG and their charge-engineering modulates normal neonatal Fc receptor binding and cellular recycling in a manner that improves therapeutic efficacy of the protein entity.

In certain embodiments, the CPM comprises a charge engineered variant of an immunoglobulin C_{H}1 and/or C_{H}L domains, or of the C_{H}3 domain. In certain embodiments, the CPM comprises a charge engineered variant of an immunoglobulin C_{H}2.

Exemplary CPMs are shown in Table 3:

| Uniprot | | charge/ | | | |
|---|---|---|---|---|---|
| ID | Protein Name | MW | MW | charge | pl |
| P06729 | T-cell surface | 0.51 | 39.45 | 20 | 9.66 |
| | antigen CD2 | | | | |
| P01732 | T-cell surface | 0.51 | 25.73 | 13 | 9.64 |
| | glycoprotein | | | | |
| | CD8 alpha chain | | | | |
| P15814 | Immunoglobulin | 0.48 | 22.96 | 11 | 10.10 |
| | lambda-like | | | | |
| | polypeptide 1 | | | | |
| P10747 | T-cell-specific | 0.48 | 25.07 | 12 | 9.46 |
| | surface | | | | |
| | glycoprotein CD28 | | | | |
| P23083 | Ig heavy chain V-I | 0.46 | 13.01 | 6 | 9.59 |
| | region V35 | | | | |
| P01730 | T-cell surface | 0.45 | 51.11 | 23 | 9.60 |
| | glycoprotein CD4 | | | | |
| P25189 | Myelin protein P0 | 0.40 | 27.55 | 11 | 9.57 |
| Q9HCN6 | Platelet | 0.30 | 36.86 | 11 | 9.35 |
| | glycoprotein VI | | | | |
| O14931 | Natural cytotoxicity | 0.23 | 21.59 | 5 | 9.17 |
| | triggering receptor 3 | | | | |
| Q9UBF9 | Myotilin | 0.20 | 55.39 | 11 | 9.18 |

In certain embodiments, the CPM is a naturally occurring human polypeptide or a domain of a naturally occurring human polypeptide, and it is selected based on the endogenous function of the full length, naturally occurring human polypeptide. Accordingly, in certain embodiments, the disclosure provides protein entities in which the CPM Portion is (i) a domain of a naturally occurring human polypeptide having surface positive charge and a net theoretic positive charge of less than +20, but for which its naturally occurring, full length human polypeptide has a net theoretic positive charge lower than the domain and (ii) the domain is from a naturally occurring human polypeptide having an endogenous, natural function In other embodiments, the CPM does not have an endogenous function as, for example, a DNA binding protein, an RNA binding protein or a heparin binding protein. In certain embodiments, the CPM does not have an endogenous function as a histone or histone-like protein. In certain embodiments, the CPM does not have an endogenous function as a homeodomain containing protein.

A CPM has tertiary structure (e.g., it is a globular protein). The presence of such tertiary structure distinguishes CPMs from unstructured, short cell penetrating peptides (CPPs) such as poly-arginine and poly-lysine and also distinguishes CPMs from cell penetrating peptides that have some secondary structure but no tertiary structure, such as penetratin and antenapedia.

In certain embodiments, the CPM is a charge-engineered immunoglobulin-based molecule. In certain embodiments, the CPM comprises an immunoglobulin region, which comprises a charge-engineered constant region (e.g., C_{H}1, C_{H}2, C_{H}3, or CL domain). In certain embodiments, the CPM comprise more than one polypeptide and at least one the polypeptide is connected to the targeting binding portion together or through a spacer region to a target binding region. In certain embodiments, the target binding region of the protein entity comprises at least variable region, such as VH or VL domain, and the CPM of the protein entity comprises at least one charge-engineered constant domain, such as at least one C_{H}1 domain, one C_{H}2 domain or one C_{H}3 domain. In some embodiments, the target binding region and the CPM are directly connected in the absence of a SR. In some embodiments, the target binding region and the CPM are directly connected in the presence of a SR.

The CH3 domain offers sites for introduction of net positive charge, such as by substitution of a negatively charged residue with a neutral or positively charged residue and/or by substitution of a neutral residue with a positively charged residue. This is an example of charge engineering the CH3 domain and, when more than one substitution is made, each is independently selected.

In certain embodiments, the residues available for substitution to increase charge are in the AB loop (residues 352-361 of the heavy chain), strand C (residues 377-382 of the heavy chain), the CD loop (residues 383-389 of the heavy chain), the EF loop (residues 414-421 of the heavy chain), strand F (residues 422-429 of the heavy chain), and/or strand G (residues 436-443 of the heavy chain).

In certain embodiments, a library of charged variants is made, based on the above, and that library is screened to identify the variants and combinations of variants the are suitable for use as CPMs.

In certain embodiments, the CPM comprises a C_{H}3 domain, particularly a C_{H}3 domain that has been altered to increase net charge, surface positive charge, and/or charge per molecular weight ratio, in certain embodiments, the CPM comprise a CH3 domain and the protein entity comprises one or more of a CL, CH1, or CH2 domain from the same antibody, but does not include the entire Fc region of the same antibody.

The disclosure contemplates all combinations of any of the foregoing aspects and embodiments with each other, as well as combinations with any of the embodiments set forth in the detailed description and examples. Any of the structural and/or functional features of the CPM may be combined with each other, as well as with any one or more of the structural and/or functional features of other components of the disclosure.

### (v) Spacer Region

The protein entity of the disclosure may comprise one or more spacer regions (SR) to connect modules of the protein entity to each other. In certain embodiments, the protein entity includes a SR connect the target-binding region and the CPM. The term “primary SR” refers to an SR that connect the target binding region and the CPM. However, one or more additional SRs may be present, depending on whether the protein entity further includes other modules, such as cargo region.

The term “spacer region,” as used herein, refers to a linking element that be can be interposed in various formats/orientations between any two modules of the protein entity, such as between the target-binding region and the CPM. The SR may be a polypeptide or peptide and may also be a chemical linker. In certain embodiments, the SR is a polypeptide or peptide, such as a flexible polypeptide or peptide. When more than one SR is present in a protein entity, the disclosure contemplates that the nature of the SR (e.g., length, sequence, etc.) is independently selected for each SR, such that the SRs may be the same or different.

When the SR is a peptide or polypeptide, its length is generally between 1 and 60 residues. However, longer SRs are also contemplated, such as SRs of about 65, 70, 75, 80, 85, 90, 95, or even about 100 residues. In certain embodiments, the SR is a flexible spacer region, such as one or more repeats of glycine and serine (Gly/Ser spacer regions). In other words, in certain embodiments, the SR comprises repeats of glycine and serine residues. Such glycine and serine linkers may also include other amino acid residues, such as cysteine residues that may provide a site for drug conjugation.

For example, in certain embodiments, the SR, whether the primary SR or another SR, comprises a formula of SₘGₙ, wherein m and n are independently selected from about 1 to about 50 and the sum of m and n is less than 50. The SR may also be represented by the formula: (SₘGₙ)ₒ, wherein m and n are independently selected from about 1 to about 50 (with the sum of m and n being less than 50), and wherein o is selected from 0 to 50. In certain embodiments the SR comprises a small globular protein.

In some embodiments, the SR is a primary SR that interconnects the target binding region and the CPM. In some embodiments, the primer SR forms a fusion protein with at least one unit of the target binding region and at least one unit of the CPM.

In some embodiments, the protein entity of the disclosure comprises more than one SR, wherein one of the SRs is a primary SR interconnecting the target binding region and the CPM and the other SRs are located within either the target binding region or the CPM. SRs located within a target binding region or a CPM are also thought of simply as “linkers” or “linker SRs”. However, such linkers may also have any of the foregoing structural features of an SR in terms of length, amino acid content, and the like. When such a linker SR is present, its length and amino acid sequence is independently selected and may be the same or different than that of other SRs present in the protein entity.

In some embodiments, one or more SRs comprise a site for small molecule conjugation. For example, an SR, such as a primary SR or another SR in the protein entity may comprise a flexible linker, such as a polypeptide linker comprises glycine and serine residues, and the flexible linker further comprises one or more sites for drug conjugation. The one or more sites for drug conjugation may comprise more than one cysteine residues interposed between at least three or more non-reactive amino acid residues. By way of further example, in certain embodiments, an SR, such as a primary SR, suitable as a site for drug conjugation comprises an amino acid sequence having the following formula:

(S₄G)₂-[Cys-(S₄G)]₄-(S₄G)₂

In some embodiments, the SR, such as the primary SR, comprises all or a portion of an immunoglobulin (Ig) comprising at least one of a C_{H}1 domain, a hinge region, a C_{H}2 domain, and a C_{H}3 domain. In certain embodiments, one or more of these Ig domains are from a human Ig, such as a human IgG1, IgG2, IgG3, or IgG4. However, the domains may also be from other Igs, such as an IgA, IgE, IgD, or IgM. In certain embodiments, the SR does not include a C_{H}3 domain of an immunoglobulin.

In certain embodiments, the SR, such as the primary SR, comprises an immunoglobulin (Ig) C_{H}1 domain. The C_{H}1 domain may be fused to a hinge region, such that the SR comprises a C_{H}1 domain and a hinge region.

In certain embodiments, the SR, such as the primary SR, comprises a C_{H}2 domain of an immunoglobulin. The SR may comprise only a C_{H}2 domain, or may comprise one or more of a C_{H}1, C_{H}2, and hinge region.

In some embodiments, the SR is devoid of general proteolytic cleavage site (PCS). In other embodiment, the SR comprises a PCS susceptible, such that the SR is susceptible to cleavage. Certain sites are cleaved only by enzyme(s) with a localization restricted to the endosome of the targeted cell. In some embodiments, the CPM may comprise a SR comprising a PCS cleavable only by enzyme(s) with a localization restricted to (i) an endosomal or lysosomal compartment, (ii) the cytoplasm, or (iii) the tumor extracellular matrix surrounding the target cell. Whether a cleavage site is present in an SR and, if so, the nature of the cleavage site is independently determined for each SR. For example, including a cleavable linker in an SR that connects a cargo region to the remainder of the protein entity permits liberation of the cargo region following some predetermined event (e.g., internalization in the target cell type).

In certain embodiments, the protein entity comprises more than one SR, and the length and sequence of each is independently selected.

Any suitable SR may be used to connect one module of a protein entity to another module or region. The disclosure contemplates protein entities comprising 0 SRs, 1 SR, such as a primary SR, and more than one SR. The nature of each SR is independently selected. Any of the features of SRs, such as those described herein and know in the art, may be combined with any of the features of the other modules of a protein entity described herein.

### (vi) Formation of Protein Entities

The present disclosure provides protein entities comprising (i) at least one target binding region; and (ii) at least one CPM and optionally at least one SR interconnecting the target binding region and the CPM. The protein entities are useful, for example, for facilitating targeted delivery and/or to enhance penetration of a therapeutic molecule (such as a cytotoxic drug) into cells expressing the cell surface target bound by the target binding region. Below are provided examples of protein entities of the disclosure and how the portions of the protein entities are associated and/or made.

As noted throughout the application, protein entities of the disclosure combine the localization to a cell of interest, via the cell surface target region with the cell penetration activity of the CPM. As a result, cell penetration of the protein entity is effected. For example, cell penetration is not ubiquitous and is preferential for cell expressing on their cell surface the cell surface target. Generally, protein entities of the disclosure provide preferential cell penetration.

Protein entities of the disclosure may combine any of the features of the various modules. Regardless of the particular category of target binding region selected, the target binding region binds a cell surface target. In the context of a protein entity, the target binding region binds the cell surface target at the cell surface, and thus contributes to penetration of the protein entity into cells.

The disclosure provides protein entities that are internalized into cells in a manner that is, in part, dependent on the binding of the target binding region to its cell surface target at the cell surface and, in part, dependent upon the cell penetration capacity of the CPM. Without being bound by theory, these protein entities promote penetration into cells with a level of specificity, and provide cell or tissue targeted delivery. In other words, generally, enhanced penetration is preferential to cells that express on the cell surface the cell surface target. Moreover, these two portions of the protein entities function cooperatively, perhaps even additively or synergistically. For example, protein entity formation (e.g., association of the target binding region with the CPM) does not inhibit the ability of the target binding region to bind the cell surface target.

Exemplary features and characteristics of protein entities of the disclosure are discussed throughout and are not necessarily repeated in this section. However, regardless of where such features are discussed, they are reflective of protein entities of the disclosure.

In certain embodiments, the protein entities of the disclosure are penetration-enhanced immunoglobulin molecules, wherein one or both of the C_{H}3 domains of the Ig are charge-engineered and function as the CPM in the protein entity. Each charge-engineered C_{H}3 domain in the protein entity can have a net positive charge of greater than 0 and less than +20, preferably greater than +3, +4, +5, +6, etc. and be capable of enhancing penetration into a target cell expressing the cell surface target. In one embodiment of this charge-engineered IgG, both C_{H}3 domains would be identical in their sequence and charge properties. Enhancement of the endosomal escape may be effected by these C-terminal C_{H}3 constant domains or an additional component may be incorporated at the C-terminus of at least one of the charge-engineered heavy chains. The penetration-enhanced immunoglobulin molecules of the present disclosure can augment endosomal escape and/or desirable intracellular trafficking for the intended therapeutic goals or an enhancer therapeutic for use with other therapeutic agents (e.g., cargo such as cytotoxic drugs).

In certain embodiments, the protein entities of the disclosure are penetration-enhanced Fab molecules, wherein either or both of the constant domains, C_{L} or C_{H}1, are charge-engineered for one domain to have a net positive charge of greater than 0 and less than +20 and are capable of enhancing penetration of Fab molecules into its target cell, and potentially augments endosomal escape. In one embodiment of this penetration-enhanced Fab (peFab), the reidues involved in enhanced positive charge could be on C_{L} or C_{H}1, or on both.

The Protein Entity of a related design may comprise a target binding region that also comprise the CPM as a component of its native structure, e.g., in a peFab in which the C_{H}1 and/or C_{L} are charge-engineered to create a penetration-enhanced Fab (peFab), or a recombinant human antibody comprising penetration-enhanced peFab in one or more positions within the protein entity (e.g., 2 peFab per IgG). Alternatively, or in addition to peFab incorporation, a recombinant human antibody is claimed that is charge-engineered to have new penetration-enhanced cell binding properties through charge engineering of the antibody C_{H}3 constant domains, unrelated to the Fv region. In another related embodiment, the IgG may have a CPM fused at one or both H chain C-termini, possibly via a flexible SR of appropriate length to effect penetration enhancement, with or without the peFab engineering.

In certain embodiments, the protein entities of the disclosure are penetration-enhanced immunoglobulin molecules, wherein the CH3 domains of the Ig are charge-engineered and function as the CPM in the protein entity. The charge-engineered CH3 domains have a net positive charge of greater than 0 and less than +20 and are capable of enhancing penetration of the immunoglobulin molecules into its target cell, e.g., into the endosome. Enhancement of the endosomal escape may be effected by these C-terminal CH3 constant domains or an additional component may be incorporated at the C-terminus of at least one of the charge-engineered heavy chains. The penetration-enhanced immunoglobulin molecules of the present disclosure can augment endosomal escape and/or desirable intracellular trafficking for the intended therapeutic goals or an enhancer therapeutic for use with other therapeutic agents (e.g., cargo such as cytotoxic drugs).

In certain embodiments, the protein entities of the disclosure are penetration-enhanced Fab molecules, wherein either or both of the constant domains, C_{L} or C_{H}1, are charge-engineered to have a net positive charge of greater than 0 and less than +20 and are capable of enhancing penetration of Fab molecules into its target cell, and potentially augments endosomal escape.

In certain embodiments, once the protein entity bound to the cell surface target enters the cell, the association between the target binding region and the cell surface target can be disrupted, and the target binding region alone can enter the endosome or lysosome.

In certain embodiments, the association between the target binding region and the CPM is disruptable. Thus, in certain embodiments, once the protein entity bound to the cell surface target enters the cell, the association between the target binding region and the CPM may be disrupted before entering the endosome. As a result, the target binding region bound to the cell surface target together enter the endosome.

In certain embodiments, once the protein entity bound to the cell surface target enters the cell, the association between the target binding region and the CPM as well as the association between the target binding region and the cell surface target may both be disrupted, and thus, the target binding region alone enters the endosome or lysosome.

However, the association need not be disrupted, and the protein entity may remain intact after entry into the cell and further into the endosome or lysosome.

Protein entities of the disclosure may, in certain embodiments, include portions in addition to the CPM and the target binding region. For example, the protein entities may include one or more spacer regions. The protein entities may include sequence that helps target the protein entity to endosome or lysosome, and/or the protein entity may include tags to facilitate detection and/or purification of the protein entity or a portion of the protein entity. These additional sequences may be located at the N-terminus, at the C-terminus or internally. Moreover, additional portions may be interconnected to the CPM to the target binding region or to both.

In certain embodiments, the CPM and the target binding regions of the protein entity are associated covalently. For example, these two portions may be fused (e.g., the protein entity comprises a fusion protein). Covalent interactions may be direct or indirect (via a spacer region). Thus, in some embodiments, such covalent interactions are mediated by one or more spacer region). In some embodiments, the spacer region is a cleavable spacer region. In certain embodiments, the cleavable spacer region comprises an amide, an ester, or a disulfide bond. For example, the spacer region may be an amino acid sequence that is cleavable by a cellular enzyme. In certain embodiments, the enzyme is a protease. In other embodiments, the enzyme is an esterase. In some embodiments, the enzyme is one that is more highly expressed in certain cell types than in other cell types. For example, the enzyme may be one that is more highly expressed in tumor cells than in non-tumor cells. In certain embodiments, the cleavable spacer region is selected or engineered to be cleavable only in the endosome. For example, the spacer region) may be more susceptible to proteases (for example, being capable of being cleaved based on relative larger sizes or lack of overall structure). In certain embodiments, specific cleavage sites might be engineered into the spacer region), for example, different cathepsin cleavage sites including cathepsin C or cathepsin K. Exemplary sequences that can be used in spacer regions and enzymes that cleave those spacer regions are presented in Table 4.

TABLE 4
Exemplary Spacer Region sequences.

| Cleavable | SEQ ID | |
|---|---|---|
| sequencer | NO: | Enzymes that Target the Spacer Region |
| X-AGVF-X | | Lysosomal thiol proteinases (see, e.g., |
| | | Duncan et al., Biosci. Rep., 2: 1041-46, |
| | | 1982; incorporated herein by |
| | | reference) |
| X-GFLG-X | | Lysosomal cysteine proteinases (see, |
| | | e.g., Vasey et al., Clin. Canc. Res., |
| | | 5: 83-94, 1999; incorporated herein by |
| | | reference) |
| X-FK-X | | Cathepsin B-ubiquitous, overexpressed in |
| | | many solid tumors, such as breast cancer |
| | | (see, e.g., Dubowchik et al., |
| | | Bioconjugate Chem., 13: 855-69, 2002; |
| | | incorporated herein by reference) |
| X-A*L-X | | Lysosomal hydrolases (see, e.g., |
| | | Trouet et al., Proc. Natl. Acad. Sci., |
| | | USA, 79: 626-29, 1982; incorporated |
| | | herein by reference) |
| X-A*LA*L-X | | Cathepsin B-ubiquitous, overexpressed |
| | | in many solid tumors, such as breast |
| | | cancer (see, e.g., Schmid et al., |
| | | Bioconjugate Chemistry, 18: 702-16, |
| | | 2007; incorporated herein by reference) |
| X-AL*AL*A-X | | Cathepsin D-ubiquitous (see, e.g., |
| | | Czerwinski et al., Proc. Natl. Acad. |
| | | Sci. USA, 95: 11520-25, 1998; |
| | | incorporated herein by reference) |

“X” denotes the CPM or the target binding region.
“*” refers to observed cleavage site.

In certain embodiments, the CPM and the target binding region are fused by using a construct that comprises an intein, which is self-spliced out to join the CPM and the target binding region via a peptide bond.

In another embodiment, e.g., where expression of a fusion construction is not practical (e.g., is inefficient) or not possible, the CPM and the target binding region are synthesized by using a viral 2A peptide construct that comprises the CPM and the target binding region for bicistronic expression. In this embodiment, the CPM and the target binding region genes may be expressed on the bicistronic construct, and the 2A peptide results in cotranslational “cleavage” of the two proteins (Trichas et al., BMC Biology 6:40, 2008).

The disclosure contemplates protein entities in which the CPM and the target binding region are associated by a covalent or non-covalent linkage. In either case, the association may be direct or via one or more additional intervening liners or moieties.

In some embodiments, a CPM and a target binding region are associated through chemical or proteinaceous linkers or spacers (e.g., a primary SR). Exemplary linkers and spacers include, but are not restricted to, substituted or unsubstituted alkyl chains, polyethylene glycol derivatives, amino acid spacers, sugars, or aliphatic or aromatic spacers common in the art.

Suitable linkers include, for example, homobifunctional and heterobifunctional cross-linking molecules. The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde.

Homobifunctional linker molecules having at least two active ester units include esters of dicarboxylic acids and N-hydroxysuccinimide Some examples of such N-succinimidyl esters include disuccinimidyl suberate and dithio-bis-(succinimidyl propionate), and their soluble bis-sulfonic acid and bis-sulfonate salts such as their sodium and potassium salts.

Heterobifunctional linker molecules have at least two different reactive groups. Examples of heterobifunctional reagents containing reactive disulfide bonds include N-succinimidyl 3-(2-pyridyl-dithio)propionate (Carlsson et al., 1978. Biochem. J., 173:723-737), sodium S-4-succinimidyloxycarbonyl-alpha-methylbenzylthiosulfate, and 4-succinimidyloxycarbonyl-alpha-methyl-(2-pyridyldithio)toluene. Examples of heterobifunctional reagents comprising reactive groups having a double bond that reacts with a thiol group include succinimidyl 4-(N-maleimidomethyl)cyclohexahe-1-carboxylate and succinimidyl m-maleimidobenzoate. Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-5N-hydroxy-succinimide ester.

Other means of cross-linking proteins utilize affinity molecule binding pairs, which selectively interact with acceptor groups. One entity of the binding pair can be fused or otherwise linked to the CPM and the other entity of the binding pair can be fused or otherwise linked to the target binding region. Exemplary affinity molecule binding pairs include biotin and streptavidin, and derivatives thereof; metal binding molecules; and fragments and combinations of these molecules. Exemplary affinity binding pairs include StreptTag (WSHPQFEK)/SBP (streptavidin binding protein), cellulose binding domain/cellulose, chitin binding domain/chitin, S-peptide/S-fragment of RNAseA, calmodulin binding peptide/calmodulin, and maltose binding protein/amylose.

In one embodiment, the CPM and the target binding region are linked by ubiquitin (and ubiquitin-like) conjugation.

The disclosure also provides nucleic acids encoding a CPM and a target binding region, such as an antibody molecule, or a non-antibody molecule scaffold, such as a DARPin, an Adnectin®, an Anticalin®, or a Kunitz domain polypeptide, or an Adhesin molecule. The protein entity of a CPM and a target binding region can be expressed as a fusion protein, optionally separated by a peptide linker. The peptide linker can be cleavable or not cleavable. A nucleic acid encoding a fusion protein can express the fusion in any orientation. For example, the nucleic acid can express an N-terminal CPM fused to a C-terminal target binding region (e.g., antibody), or can express an N-terminal target binding region fused to a C-terminal CPM.

A nucleic acid encoding a CPM can be on a vector that is separate from a vector that carries a nucleic acid encoding a target binding region. The CPM and the target binding region can be expressed separately, and interconnected (including chemically linked) prior to administration for binding a cell surface target. The isolated protein entity can be formulated for administration to a subject, as a pharmaceutical composition.

The disclosure also provides host cells comprising a nucleic acid encoding the CPM or the target binding region, or comprising the protein entity as a fusion protein. The host cells can be, for example, prokaryotic cells (e.g., E. coli) or eukaryotic cells.

In certain embodiments, the recombinant nucleic acids encoding a protein entity, or the portions thereof, may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding a protein entity of the disclosure (e.g., a protein entity comprising a CPM and a target binding region) polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

The disclosure also provides host cells comprising or transfected with a nucleic acid encoding the protein entity as a fusion protein. The host cells can be, for example, prokaryotic cells (e.g., E. coli) or eukaryotic cells. Other suitable host cells are known to those skilled in the art.

In addition to the nucleic acid sequence encoding the protein entity or portions of the protein entity, a recombinant expression vector may carry additional nucleic acid sequences, such as sequences that regulate replication of the vector in a host cells (e.g., origins of replication) and selectable marker genes. The selectable marker gene facilitates selection of host cells into which the vector has been introduced. Exemplary selectable marker genes include the ampicillin and the kanamycin resistance genes for use in E. coli.

The present disclosure further pertains to methods of producing fusion proteins of the disclosure. For example, a host cell transfected with an expression vector can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides. In a preferred embodiment, the polypeptide is a fusion protein containing a domain which facilitates its purification.

A nucleic acid encoding a CPM can be on a vector that is separate from a vector that carries a nucleic acid encoding a target binding region. The portions of the protein entity can be expressed separately, and connected prior to administration to binding a cell surface target. The isolated protein entity can be formulated for administration to a subject, as a pharmaceutical composition.

Recombinant nucleic acids of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

It should be understood that fusion polypeptides or protein of the present disclosure can be made in numerous ways. For example, a CPM and a target binding region can be made separately, such as recombinantly produced in two separate cell cultures from nucleic acid constructs encoding their respective proteins. Once made, the proteins can be chemically conjugated directly or via a linker. By way of another example, the fusion polypeptide can be made as an inframe fusion in which the entire fusion polypeptide, optionally including one or more linker, tag or other moiety, is made from a nucleic acid construct that includes nucleotide sequence encoding both a CPM and a target binding region of the protein entity.

In certain embodiments, a protein entity of the disclosure is formed under conditions where the linkage (e.g., by a covalent or non-covalent linkage) is formed, while the activity of the target binding region is maintained.

To minimize the effect of linkage on target binding region activity (e.g., target binding), any linkage to the target binding region can be at a site on the protein that is distant from the target-interacting region of the target binding region.

Further, in the case of a cleavable linker, an enzyme that cleaves a linker between the a CPM and a target binding region does not have an effect on the target binding region, such that the structure of the target binding region remains intact and the target binding region retains its target binding activity.

In other embodiments, the CPM and the target binding regions of the protein entity are separated, e.g., within the cell, under conditions where the linkage (e.g., a covalent or non-covalent linkage) is dissociated, while the activity of the target binding region is maintained. For example, the CPM and target binding region can be joined by a cleavable peptide linker that is subject to a protease that does not interfere with activity of the target binding region.

In some embodiments the CPM and target binding region are separated in the endosome due to the lower pH of the endosome. Thus in these embodiments, the linker is cleaved or broken in response to the lower pH, but the activity of the target binding region is not affected.

In some embodiments the CPM and the target binding region remain intact in the endosome despite the lower pH of the endosome. The target binding region is engineered or selected to remain bound to the cell surface target in the presence of the lower pH of the endosome as well as in the extracellular environment.

In some embodiments, the target binding region binds and/or inhibits activity of the cell surface target while the target binding region is still connected with the CPM. Thus the protein entity does not dissociate after administration to the subject, prior to the binding between the target binding region on the cell surface target protein. While in other embodiments, the CPM and target binding region may dissociate following delivery of the cell surface target into the cell and, for example, the target binding region may still bind to its cell surface target inside the cell after dissociation from the CPM.

It should be noted that the disclosure contemplates that the foregoing description of protein entities is applicable to any of the embodiments and combinations of embodiments described herein. For example, the description is applicable in the context of protein entities in which the target binding region is associated with a portion comprising a CPM presented in the context of additional sequence, such as additional sequence from its own naturally occurring polypeptide. In this context, any interconnection is via the two portions of the protein entity (the target binding region and the CPM), but the interconnection may not be directly between the CPM and the target binding region.

### (vii) Cargo

The disclosure provides protein entities that are internalized into cells in a manner that is, in part, dependent on the binding of the target binding region to its cell surface target at the cell surface and, in part, dependent upon the cell penetration capacity of the CPM. Without being bound by theory, these protein entities promote penetration into cells with a level of specificity, and provide cell or tissue targeted delivery. In other words, generally, enhanced penetration is preferential of cells that express on the cell surface the cell surface target. Moreover, these two portions of the protein entities function cooperatively, perhaps even additively or synergistically. For example, protein entity formation (e.g., association of the target binding region with the CPM) does not inhibit the ability of the target binding region to bind the cell surface target. In some cases, the dissociation constant or avidity of the target binding region for the cell surface target is approximately the same, or even improved (e.g., lower K_{D}) in the context of the protein entities in comparison to when the target binding region is present alone (e.g., in the absence of the CPM). Similarly, the CPM retains its ability for delivery into cells and tissues. In certain embodiments, these protein entities can also be used for delivering a cargo into cells. The protein entity of the disclosure can be associated with a cargo region, such as a protein, peptide, or small organic molecule. In certain embodiments, the cargo region may be conjugated (e.g., fused or linked) to the protein entity for targeted delivery. In certain embodiments, administration of the conjugated protein entity and cargo region achieves a better therapeutic effect or activity level than administration of the cargo portion alone.

In certain embodiments, the cargo portion may be co-administered with the protein entity in trans for targeted delivery. Co-administration of the protein entity and cargo portion in trans achieves a better therapeutic effect or activity level than administration of the cargo portion alone. Without being bound by theory, even when the cargo region is co-administered in trans, the protein entity may help to increase the effective amount of cargo region available in the cytoplasm or nucleus of the cell. This would occur in a target protein, consistent with the targeted delivery of the protein entity.

Regardless of whether cargo is appended to the protein entity or delivered in trans, generally, the cargo is one with therapeutic or cell modulating activity that requires transport into cells to achieve the therapeutic effect or modulation. Below various categories of cargo, as well as specific examples of cargo are described. These specific examples of cargo are merely illustrative. We note that, depending on the cargo, the cargo may be appended to the protein entity in any of a variety of ways. Exemplary methodologies are described herein, however, any suitable approach that appends the cargo to the protein entity without negatively impacting the activity of the cargo (or of the module to which the cargo is appended) is contemplated. For example, when the cargo is a protein or peptide, the cargo may be appended to the protein entity via a SR that is a flexible polypeptide or peptide linker, such as to form a fusion protein with at least one unit of a CPM or a target binding region. When the cargo is a small molecule, such as a drug, the cargo may be chemically conjugated, such as via reactive cysteine or lysine residues. This conjugation may be via any module, such as the target binding region, the primary SR, or the CPM. In certain embodiments, the small molecule (e.g., drug, such as a cytotoxic drug) is appended via a drug conjugation site in the primary SR. In certain embodiments, the 1, 2, 3, or 4 molecules of drug are appended to each molecule of protein entity, such as via one or more drug conjugation sites in the primary SR.

Small Organic Molecules

Virtually any small molecule, such as a small organic or inorganic molecule, can be conjugated (e.g., appended or linked) to the protein entity of the present disclosure. In certain embodiments, the small molecule is a small organic molecule. In certain embodiments, the small molecule is less than 1000, less than 750, less than 650, or less than 550 amu. In other embodiments, the small molecule is less than 500 amu, less than 400 amu, or less than 250 amu.

In certain embodiments, the suitable small molecule is a cytotoxic agent, such as auristatin, calicheamicin, maytansinoid, anthracycline, pseudomonas exotoxin (e.g., PE38 or PE40, shortened forms typically used in conjugation with antibodies), ricin toxin (e.g., Deglycosylated A chain or dgA), and diphtheria toxin, or derivative or analogs thereof. Appending these or other cytotoxic agents to a protein entity of the disclosure is useful for generating targeted drug conjugates—akin to antibody-drug conjugates available. However, unlike available antibody-drug conjugates, protein entities of the disclosure have enhanced cell penetration activity, cell targeting function, and may even help facilitate effective delivery of the appended drug to the cytosol and/or nucleus of the cell.

The foregoing cytotoxic agents are merely exemplary of small molecule cargo. Also contemplated are other chemotherapeutics, regardless of mechanisms of action, other agents that promote cell death, inhibit cell survival, or inhibit cell proliferation.

In certain embodiments, it is advantageous to prevent the small molecule from crossing the blood-brain bather. Conjugation to a protein would be useful to prevent the small molecule from crossing the blood-brain barrier. However, the molecule would still be available to other tissues. This would help decrease off target affect on the brain, and thus, improve the safety of the delivered small molecule agent.

Exemplary small molecules include, but are not limited to methotrexate (for treating autoimmune diseases), small molecules for delivery to liver, such as therapies for hepatitis (e.g., telaprevir and boceprevir for HCV and entecavir or lamivudine for HBV).

Further exemplary small molecules include chemotherapeutics or other small molecules for treating cancer. A particular example of a small molecule useful for liver and kidney cancers is sorafenib.

A particular example of small molecules where it would be advantageous to limit crossing of the blood-brain barrier are platelet inhibitors, such as integrilin or aggrastat. Limiting access to the blood brain barrier is useful for preventing intracerebral bleeding.

The foregoing are merely exemplary of the small molecules (including organic and inorganic molecules that can be used as a cargo region) that may be delivered with targeting specificity using a protein entity of the disclosure.

As discussed below, small molecules and other cargos can also be delivered in trans (e.g., not appended to) with the protein entity. Any of the exemplary small molecules described herein may also be so delivered.

Proteins and Peptides

In certain embodiments, the cargo region of the protein entity is a protein or peptide. Exemplary categories of proteins and peptides that may serve as cargo are described in more detail below. However, the disclosure contemplates that virtually any protein or peptide can be used as the cargo region of a protein entity of the disclosure. For example, the protein or peptide may be one that, under naturally occurring circumstances would be functional in a specific tissue, and delivery is useful for augmenting or replacing activity that is supposed to be endogenously active in one or both of those tissues. By way of further example, the protein or peptide may be one designed to inhibit activity of a target that is expressed or misexpressed in the target tissue, and delivery is useful for inhibiting that activity. In certain embodiments, the cargo region is a polypeptide or peptide but does not include an antibody or antibody mimic. In certain embodiments, the cargo region does not include an enzyme. In certain embodiments, the cargo region does not include a transcription factor.

Enzymes

In certain embodiments, the cargo region comprises an enzyme. Without being bound by theory, protein entities in which the cargo region is an enzyme are suitable for enzyme replacement strategies in which subjects are unable to produce an enzyme having proper activity (at all or, at least, in sufficient quantities) necessary for normal function and, in some case, essential for life.

When provided as a protein entity with the target-binding region and the CPM, the enzyme portion (cargo region comprising an enzyme) is delivered into cells where it can provide needed enzymatic activity. Advantageously, appending the enzyme to the core protein entity to form a protein entity comprising an enzyme permits targeted (e.g., non-ubiquitous) delivery of the enzyme.

An enzyme is a protein that can catalyze the rate of a chemical reaction within a cell. Enzymes are long, linear chains of amino acids that fold to produce a three-dimensional product having an active site containing catalytic amino acid residues. Substrate specificity is determined by the properties and spatial arrangement of the catalytic amino acid residues forming the active site.

As used herein an “enzyme” refers to a biologically active enzyme. The term “enzyme” further refers to “simple enzymes” which are composed wholly of protein, or “protein entity enzymes”, also referred to as “holoenzymes” which are composed of a protein component (the “apozyme”) and a relatively small organic molecule (the “co-enzyme”, when the organic molecule is non-covalently bound to the protein or “prosthetic group”, when the organic molecule is covalently bound to the protein).

As used herein the term an “enzyme” also refers to a gene for an enzyme and includes the full-length DNA sequence, a fragment thereof or a sequence capable of hybridizing thereto.

Classification of enzymes is conventionally based on the type of reaction catalyzed.

In certain embodiments of the disclosure the enzyme is selected from the group consisting of: a kinase, a phosphatase, a ligase, an oxidoreductase, a transferase, a hydrolase, a hydroxylase, a lyase, an isomerase, a dehydrogenase, an aminotransferase, a hexosamidase, a glucosidase, or a glucosyltransferase, a phenyalanine hydroxylase. The categories of enzymes are well known in the art and one of skill in the art can readily envision one or more examples of each category of enzyme. For example, the enzyme is a phenyalanine hydroxylase. The protein entity associated with the phenyalanine hydroxylase can be used to treat or alleviate the symptoms associated with phenylketonuria (PKU).

To illustrate, a brief description of these categories of enzymes is provided. “Oxidoreductases” catalyze oxidation-reduction reactions. “Transferases” catalyze the transfer of a group (e.g a methyl group or a glycosyl group) from a donor compound to an acceptor compound. “Hydrolases” catalyze the hydrolytic cleavage of C—O, C—N, C—C and some other bonds, including phosphoric anhydride bonds. “Hydroxylases” catalyze the formation of a hydroxyl group on a substrate by incorporation of one atom (monooxygenases) or two atoms (dioxygenases) of oxygen. “Lyases” are enzymes cleaving C—C, C—O, C—N, and other bonds by elimination, leaving double bonds or rings, or conversely adding groups to double bonds. “Isomerases” catalyse intra-molecular rearrangements and, according to the type of isomerism, they may be called racemases, epimerases, cis-trans-isomerases, isomerases, tautomerases, mutases or cycloisomerases. “Ligases” catalyze bond formation between two compounds using the energy derived from the hydrolysis of a diphosphate bond in ATP or a similar triphosphate in ATP.

Other categories of enzymes, characterized by their substrate rather than the type of reaction catalyzed include the following: an enzyme that degrades glycosaminoglycans, glycolipids, or sphingolipids; an enzyme that degrades glycoproteins; an enzyme that degrades amino acids; an enzyme that degrades fatty acids; or an enzyme involved in energy metabolism. These categories of enzymes may, in some cases, overlap with the categories of enzymes described based on reaction catalyzed. Regardless of whether described based on substrate, reaction catalyzed, or both, one of skill in the art can readily envision examples of these classes of enzymes. Any of these are suitable for use in the present disclosure as a cargo region. In certain embodiments, of any of the foregoing, the enzyme is a human enzyme (e.g., an enzyme that is typically expressed endogenously in humans). In certain embodiments, the enzyme is a mammalian enzyme.

In certain embodiments, an enzyme for use as a cargo region in the present disclosure is not a ligase. In certain embodiments, an enzyme for use as a cargo region in the present disclosure is not a kinase. In certain embodiments, an enzyme for use as a cargo region in the present disclosure is not a recombinase.

Enzymes can function intracellularly or extracellularly. Intracellular enzymes are those whose endogenous function is inside a cell, such as in the cytoplasm or in a specific subcellular organelle. Such enzymes are responsible for catalyzing the reactions in the cellular metabolic pathways, for example, glycolysis. In the context of the present disclosure, delivery of intracellular enzymes is particularly preferred. In certain embodiments of the disclosure, the enzyme moiety is specifically targeted to an intracellular organelle in which the wild-type enzyme is constitutively or inducibly expressed.

In certain embodiments of the disclosure, the enzyme is a “kinase”, which catalyzes phosphoryltransfer reactions in all cells. Kinases are particularly prominent in signal transduction and co-ordination of protein entity functions such as the cell cycle. Non-limiting examples include tyrosine kinases, deoxyribonucleoside kinases, monophosphate kinases and diphosphate kinases.

In certain embodiments, the enzyme is a “dehydrogenase”. Dehydrogenases catalyze the removal of hydrogen from a substrate and the transfer of the hydrogen to an acceptor in an oxidation-reduction reaction. Widely implemented in the citric acid cycle, also referred to as the tricarboxylic acid cycle (TCA cycle) or the Krebs cycle, in which energy is generated in the matrix of the mitochondria through the oxidation of acetate derived from carbohydrates, fats and protein into carbon dioxide and water. Non-limiting examples of dehydrogenases include, medium-chain-acyl-CoA-dehydrogenase, very long-chain-acyl-CoA-dehydrogenase and isobutyryl-CoA-dehydrogenase.

In certain embodiments, the enzyme is an “aminotransferase” or “transaminase”. Such enzymes catalyze the transfer of an amino group from a donor molecule to a recipient molecule. The donor molecule is usually an amino acid while the recipient (acceptor) molecule is usually an alpha-2 keto acid.

In certain embodiments, the cargo region is an enzyme. For example, the enzyme may be a human protein endogenously expressed in humans. Alternatively, the enzyme may be a non-human protein and/or a protein that is not endogenously expressed in humans.

Exemplary categories of enzymes suitable for use as cargo are: kinases, phosphatases, ligases, proteases, oxidoreductases, transferases, hydrolases, hydroxylases, lyases, isomerases, dehydrogenases, aminotransferases, hexosamidases, glucosidases, or glucosyltransferases. Thus, in certain embodiments, the cargo is an enzyme selected from the group consisting of a kinase, a phosphatase, a ligase, a protease, an oxidoreductase, a transferase, a hydrolase, a hydroxylase, a lyase, an isomerase, a dehydrogenase, an aminotransferase, a hexosamidase, a glucosidase, or a glucosyltransferase. In certain embodiments, the enzyme is a human enzyme endogenously expressed in human subjects.

Further exemplary categories of enzymes are: an enzyme that degrades glycosaminoglycans, glycolipids, or sphingolipids; an enzyme that degrades glycoproteins; an enzyme that degrades amino acids; an enzyme that degrades fatty acids; or an enzyme involved in energy metabolism. In certain embodiments, the enzyme is a human enzyme endogenously expressed in human subjects.

In certain embodiments, the enzyme is not a recombinase and/or is not a non-human protein.

In certain embodiments, the enzyme is a thymidine kinase, such as HSV-TK or a variant thereof.

The understanding in the art of enzymes is high, and examples of various human enzymes abound in the scientific and lay literature. One of skill in the art can select the appropriate enzyme and can readily obtain its amino acid sequence.

The disclosure contemplates that sometimes a particular protein is not itself an enzyme, but is necessary for enzymatic or other catalytic or functional activity. Accordingly, in certain embodiments, the cargo region comprises a co-factor, accessory protein, or member of a multi-protein protein entity. Preferably, such a co-factor, accessory protein, or member of a multi-protein protein entity is a human protein or peptide. The protein or peptide should maintain its ability to bind to its endogenous cognate binding partners when provided as part of a protein entity (provided that for embodiments in which the protein entity is disrupted after cell penetration, the protein or peptide should maintain its ability to bind to its endogenous cognate binding partner(s) before and/or after protein entity disruption).

Tumor Suppressors

A tumor suppressor or anti-oncogene protects a cell from at least one step on the path to disregulated cell behavior, such as occurs in cancer. Mutations that result in a loss or decrease in the expression or function of a tumor suppressor protein can lead to cancer. Sometimes such a mutation is one of multiple genetic changes that ultimately lead to disregulated cell behavior. As used herein, a “tumor suppressor protein” or “tumor suppressor” is a protein, the loss of or decrease in expression and/or function of which, increases the likelihood of or ultimately leads to unregulated or disregulated cell proliferation, migration, or other changes indicative of hyperplastic or neoplastic transformation.

Unlike oncogenes, tumor suppressor genes often, although not exclusively, follow the “two-hit”, which implies that both alleles that code for a particular protein must be affected before a phenotype is discernable. This is because if only one allele for the gene is damaged, the second can sometimes still produce the correct protein in an amount sufficient to maintain proper function. There are exceptions to the “two-hit” model for tumor suppressors. For example, certain mutations in some tumor suppressors can function as a “dominant negative”, thus preventing the normal functioning of the protein produced from the wild type allele. Other examples include tumor suppressors that exhibit haploinsufficiency, such as patched (PTCH). Tumor suppressors that exhibit haploinsufficiency are sensitive to decreased levels or activity, such that even reduction in function following mutation in one allele is sufficient to result in a discernable phenotype.

Functional tumor suppressor proteins either have a dampening or repressive effect on the regulation of the cell cycle or promote apoptosis, and sometimes do both. Exemplary endogenous functions for tumor suppressor proteins generally fall into categories, such as the following:
Some tumor suppressor proteins repress the activity or expression of proteins or genes essential for continuing the cell cycle. In the absence of control by the tumor suppressor, the cell cycle may continue unchecked—leading to inappropriate cell division.
Some tumor suppressor proteins function to couple the cell cycle to DNA damage, such that the cell cycle will arrest if there is DNA damage and will only continue if that damage can be repaired. In the absence of control by the tumor suppressor, cells can divide in the presence of damaged DNA.
Some tumor suppressors are also referred to as metastasis suppressors because of their role in cell adhesion, which functions to prevent tumor cells from dispersing and losing contact inhibition properties. In the absence of this control, the risk and extent of metastasis increases.
Some tumor suppressors function as DNA repair proteins.

There are numerous examples of tumor suppressor proteins belonging to any one or more of the foregoing classes, as well as tumor suppressors that can be separately characterized. One of skill in the art can readily envision numerous proteins characterized as tumor suppressor proteins. Exemplary tumor suppressor proteins include, but are not limited to, p53, p16, patched (PTCH), and ST5. The disclosure contemplates that any tumor suppressor protein, including any of these specific tumor suppressor proteins and/or any of the foregoing category(ies) of tumor suppressor proteins are suitable for use as the cargo region in the protein entities of the disclosure.

In certain embodiments, the cargo region (the tumor suppressor portion) does not include a transcription factor. In other words, in certain embodiments, the tumor suppressor protein is not also a transcription factor. In certain embodiments, the tumor suppressor portion does not include p53.

Protein entities of the disclosure are useful for delivering a tumor suppressor protein to cells and tissues in vitro or in vivo. In certain embodiments, delivery is for augmenting or replacing missing or decreased function or expression of the endogenous tumor suppressor protein. Thus, although the function or expression of the tumor suppressor protein may not be decreased in all cells and tissue in culture or in an organism, the disclosure contemplates that the protein entities deliver tumor suppressor protein to cells and tissue—at least a portion of which are characterized by decreased or missing function or expression of that tumor suppressor protein. In certain embodiments, the decreased or missing function and/or expression is due, at least in part, to a mutation in the gene encoding the tumor suppressor protein. In certain embodiments, the decreased or missing function and/or expression is not due to a mutation in the gene encoding the tumor suppressor protein.

To further describe the tumor suppressor portion of the protein entities of the disclosure, exemplary tumor suppressor proteins are described below.

Patched (PTCH)

Protein patched homolog 1 (patched or PTCH) is encoded by the ptch1 gene and is a tumor suppressor protein. Mutations of this gene have been associated with nevoid basal cell carcinoma syndrome, basal cell carcinoma, medulloblastoma, esophageal squamous cell carcinoma, transitional cell carcinomas of the bladder, and rhabdomyosarcoma. Moreover, hereditary mutations in PTCH cause Gorlin syndrome, an autosomal dominant disorder. In addition, misregulation of this tumor suppressor protein can lead to other defects of growth regulation, such as holoprosencephaly and cleft lip and palate.

Given the role of PTCH as a tumor suppressor protein, in certain embodiments, protein entities of the disclosure comprise PTCH or a functional fragment thereof. In other words, the tumor suppressor portion of the protein entity comprises, in certain embodiments, PTCH (such as human PTCH) or a functional fragment thereof.

ST5

Suppression of tumorigenicity 5 is a protein that in humans is encoded by the ST5 gene. This gene was identified by its ability to suppress the tumorigenicity of Hela cells in nude mice. The protein encoded by this gene contains a C-terminal region that shares similarity with the Rab 3 family of small GTP binding proteins. ST5 protein preferentially binds to the SH3 domain of c-Abl kinase, and acts as a regulator of MAPK1/ERK2 kinase, which may contribute to its ability to reduce the tumorigenic phenotype in cells.

Three alternatively spliced transcript variants of this gene encoding distinct isoforms exist. In certain embodiments, the cargo region comprises ST5 or a functional fragment thereof. Isoform 3 (p70) of ST5 (see www.uniprot.org/uniprot/P78524) has been shown to restore contact inhibition in mouse fibroblast cell lines. Accordingly, in certain embodiments, the cargo region of a protein entity of the disclosure comprises isoform 3 of ST5, preferably isoform 3 of human ST5.

ST5 was found downregulated following LH and FSH stimulation of human granulosa cells which comprise the main bulk of the ovarian follicular somatic cells. Rimon et al., Int J Oncol. 2004 May; 24(5):1325-38. Without being bound by theory, given that hypergonadotropin stimulation is believed to increase risk for ovarian cancer, administration of ST5 protein may help offset this down regulation. In such a context, ST5 administration may be useful not only as a therapeutic, but also as a prophylactic measure. However, therapeutic use in ovarian cancer is just one example. Given the tumor suppressor function of ST5, the disclosure contemplates providing ST5 in any context characterized to decreased expression and/or function of or mutation in ST5.

P16

p16 is a tumor suppressor protein and, in certain embodiments, protein entities of the disclosure are useful for delivering a tumor suppressor protein, specifically p16 or a functional fragment thereof, to cells and tissues in vitro or in vivo. In other words, in certain embodiments, the cargo region comprises p16 or a functional fragment thereof. In certain embodiments, delivery is for augmenting or replacing missing or decreased function or expression of endogenous p16 protein. Thus, although the function or expression of the tumor suppressor protein may not be decreased in all cells and tissue in culture or in an organism, the disclosure contemplates that the protein entities deliver tumor suppressor protein to cells and tissue—at least a portion of which are characterized by decreased or missing function or expression of that p16 tumor suppressor protein. In certain embodiments, the decreased or missing function and/or expression is due, at least in part, to a mutation in the gene encoding p16 tumor suppressor protein. In certain embodiments, the decreased or missing function and/or expression is not due to a mutation in the gene encoding p16 tumor suppressor protein.

Tumor suppressors for use in the protein entities of the disclosure comprise, in certain embodiments, p16, or a functional fragment thereof. The full length amino acid sequence of human p16 is set forth below:

MEPAAGSSMEPSADWLATAAARGRVEEVRALLEAGALPNAPNSYGRRPIQ
VMMMGSARVAELLLLHGAEPNCADPATLTRPVHDAAREGFLDTLVVLHRA
GARLDVRDAWGRLPVDLAEELGHRDVARYLRAAAGGTRGSNHARIDAAEG
PSDIPD.

Cyclin-dependent kinase inhibitor 2A, (CDKN2A, p16^{Ink4A}) is a tumor suppressor protein that, in humans, is encoded by the CDKN2A gene. This tumor suppressor protein is commonly referred to in the art and will be referred to herein as “p16” or “p16Ink4”. p16 plays an important role in regulating the cell cycle, and mutations in p16 increase the risk of developing a variety of cancers.

p16 has 5 isoforms (www.uniprot.org/uniprot/P42771), however, isoform 4 is a completely different protein arising from an alternate reading frame and expression of isoform 5 is generally undetectable in non-tumor cells. Isoforms 1, 2, 3, and 5 bind to CDK4/6 and are of interest and may be useful as the p16 portion of the protein entities of the disclosure. A full length amino acid sequence of isoform 1 of human p16 (often referred to as the canonical p16 amino acid sequence) is of particular interest and is set forth above. Isoform 2 is essentially a functional fragment of this canonical sequence—missing amino acids 1-51 relative to isoform 1. Isoform 3 is expressed specifically in the pancreas and, in certain embodiments, may be used to replace p16 function in subjects with a pancreatic tumor. The term “p16 tumor suppressor protein” or p16 refers to isoform 1, 2, 3, or 5 of p16, unless a specific isoform or sequence is specified. In certain embodiments, isoform 1 of human p16 (a protein having the amino acid sequence set forth above) is used in a protein entity of the disclosure. In certain embodiments, the p16 portion comprises or consists of an amino acid sequence at least 80%, 85%, 90%, 91%, 92%, 93%, 94%, 95%, 96%, 97%, 98%, 99%, or 100% identical to SEQ ID NO: 5. Regardless of the particular p16 protein used in the protein entity, the protein must retain p16 bioactivity, such as the functions of p16 described herein and known in the art (e.g., binding to CDK6; ability to inhibit cyclin D-CDK4 kinase activity, etc.).

The CDKN2A gene generates several transcript variants that differ in their first exons. At least three alternatively spliced variants encoding distinct proteins have been reported, two of which encode structurally related isoforms known to function as inhibitors of CDK4. The remaining transcript includes an alternate exon 1 located 20 kilobases upstream of the remainder of the gene. This transcript contains an alternative open reading frame (ARF) that specifies a protein that is structurally unrelated to the products of the other variants. The ARF product functions as a stabilizer of the tumor suppressor protein p53. In spite of their structural and functional differences, the CDK inhibitor isoforms and the ARF product encoded by this gene, through the regulatory roles of CDK4 and p53 in cell cycle progression, share a common functionality in control of the G1 phase of the cell cycle. This gene is frequently mutated or deleted in a wide variety of tumors and is known to be an important tumor suppressor gene.

The present disclosure provides protein entities comprising a p16 tumor suppressor protein, or a functional fragment or functional variant thereof, associated with a CPM portion. In certain embodiments, the CPM portion and/or the protein entity does not include a protein that is an endogenous substrate or binding partner for p16. In certain embodiments, the protein entity comprising a CPM portion and a p16 portion does not include a transcription factor. In certain embodiments, the protein entity does not include p53.

Protein entities of the disclosure are useful for delivering a tumor suppressor protein, specifically p16 or a functional fragment thereof, to cells and tissues in vitro or in vivo. In certain embodiments, delivery is for augmenting or replacing missing or decreased function or expression of endogenous p16 protein. Thus, although the function or expression of the tumor suppressor protein may not be decreased in all cells and tissue in culture or in an organism, the disclosure contemplates that the protein entities deliver tumor suppressor protein to cells and tissue—at least a portion of which are characterized by decreased or missing function or expression of that p16 tumor suppressor protein. In certain embodiments, the decreased or missing function and/or expression is due, at least in part, to a mutation in the gene encoding p16 tumor suppressor protein. In certain embodiments, the decreased or missing function and/or expression is not due to a mutation in the gene encoding p16 tumor suppressor protein.

Tumor suppressors for use in the protein entities of the disclosure comprise p16, or a functional fragment or functional variant thereof. Cyclin-dependent kinase inhibitor 2A, (CDKN2A, p16^{Ink4A}) is a tumor suppressor protein that, in humans, is encoded by the CDKN2A gene. This tumor suppressor protein is commonly referred to in the art and will be referred to herein as “p16” or “p16Ink4”. p16 plays an important role in regulating the cell cycle, and mutations in p16 increase the risk of developing a variety of cancers. The full length amino acid sequence of human p16, isoform 1 is set forth in SEQ ID NO: 5.

The disclosure contemplates the use of p16, such as human p16. In certain embodiments, the p16 portion comprises a full length, native p16 protein. However, variants of native p16 that retain function (e.g., functional variants) can also be used. Exemplary variants retain the activity of p16 (e.g., retain greater than 50%, preferably greater than 70% of the native activity) and include 1, 2, 3, 4, 5, 6, 7, 8, 9, or 10 amino acid substitutions, deletions, or additions relative to the native p16 sequence. Each such change is independently selected (e.g., each substitution is independently selected). Further exemplary variants retain the activity of p16 and comprise an amino acid sequence at least 80%, 85%, 90%, 91%, 92%, 93%, 94%, 95%, 96%, 97%, 98%, 99%, or greater than 99% identical to the amino acid sequence set forth above. Functional variants may also be a functional variant of a functional fragment of p16. Functional variants or the full length or fragment of native p16 also include variants, such as amino acid additions, deletions, substitutions, or truncations intended to increase protein stability improve biochemical or biophysical characteristics, or improve binding to CDK4 and/or CDK 6.

Contemplated functional fragments include fragments comprising: a fragment of p16 lacking the first ankyrin repeat, native isoform 2, residues 10 to 134 of the sequence set forth above, and residues 10 to 101 of the sequence set forth above.

The p16 portion may be phosphorylated either during protein entity formation or in a post-production step. In certain embodiments, the p16 portion is not phosphorylated or is under phosphorylated (e.g., less phosphorylated then native p16). In certain embodiments, the p16 portion is hyper-phosphorylated (e.g., more phosphorylated then native p16).

Since its discovery as a CDKI (cyclin-dependent kinase inhibitor) in 1993, the importance in cancer of the tumor suppressor p16 (INK4A/MTS-1/CDKN2A) has gained widespread appreciation. The frequent mutations and deletions of p16 in human cancer cell lines first suggested an important role for p16 in carcinogenesis. This genetic evidence for a causal role was significantly strengthened by the observation that p16 was frequently inactivated in familial melanoma kindreds. Since then, a high frequency of p16 gene alterations were observed in many primary tumors.

In human neoplasms, p16 is silenced in at least three ways: homozygous deletion, methylation of the promoter, and point mutation. The first two mechanisms comprise the majority of inactivation events in most primary tumors. Additionally, the loss of p16 may be an early event in cancer progression, because deletion of at least one copy is quite high in some premalignant lesions. p16 is a major target in carcinogenesis, rivaled in frequency only by the p53 tumor-suppressor gene. Its mechanism of action as a CDKI has been elegantly elucidated and involves binding to and inactivating the cyclin D-cyclin-dependent kinase 4 (or 6) protein entity, and thus renders the retinoblastoma protein inactive. This effect blocks the transcription of important cell-cycle regulatory proteins and results in cell-cycle arrest.

Mutations in the CDKN2A gene and other factors that decrease the expression and/or function of a p16 protein isoform correlate with increased risk of a wide range of cancers. Exemplary cancers often associated with mutations or alterations in p16 include, but are not limited to, melanoma, pancreatic ductal adenocarcinoma, gastric mucinous cancer, primary glioblastoma, mantle cell lymphoma, hepatocellular carcinoma and ovarian cancer. Additionally, mutations or deletions in p16 are frequently found in, for example, esophageal and gastric cancer cell lines.

p16 misregulation is implicated in numerous cancers. Once such cancer is ovarian cancer, where the cancers of greater than half the patients have p16 misregulation. Accordingly, in certain embodiments, p16 portion protein entities of the disclosure are particularly suitable for treating and studying ovarian cancer, as well as metastases from primary ovarian cancer. Additional discussion on ovarian cancer and p16 is provided below by way of a specific example of a cancer that could be treated and studied using the protein entities of the disclosure. This is not meant to limit the claims, but merely to provide an example of a p16 deficient cancer that could be studied and/or treated.

Ovarian cancer is the most lethal of the gynecological malignancies. Novel-targeted therapies are needed to improve outcomes in ovarian cancer patients, where 75% of patients present with advanced (stage III or IV) disease. Although more than 80% of women treated benefit from first-line therapy, tumor recurrence occurs in almost all these patients at a median of 15 months from diagnosis (Hennessy B T, Coleman R L, Markman M. Ovarian cancer. Lancet 2009; 374:1371-8).

Cell cycle dysregulation is a common molecular finding in ovarian cancer. Under normal control, the cell cycle functions as a tightly regulated process consisting of several distinct phases. Progression through the G1-S phase requires phosphorylation of the retinoblastoma (Rb) protein by CDK4 or CDK6 (Harbour et al. Cdk phosphorylation triggers sequential intramolecular interactions that progressively block Rb functions as cells move through G1. Cell 1999; 98: 859-69; Lundberg A S, Weinberg R A. Functional inactivation of the retinoblastoma protein requires sequential modification by at least two distinct cyclin-cdk protein entities. Mol Cell Biol 1998; 18:753-61; Chen et al. Overexpression of Cdk6-cyclin D3 highly sensitizes cells to physical and chemical transformation. Oncogene 2003; 22:992-1001) in protein entity with their activating subunits, the D type cyclins, D1, D2, or D3 (Meyerson M, Harlow E. Identification of G1 kinase activity for cdk6, a novel cyclin D partner. Mol Cell Biol 1994; 14:2077-86). Hyperphosphorylation of Rb diminishes its ability to repress gene transcription and consequently allows synthesis of several genes that encode proteins, which are necessary for DNA replication (Harbour J W, Dean D C. The Rb/E2F pathway: expanding roles and emerging paradigms. Genes Dev 2000; 14:2393-409).

Deregulation of the CDK4/6-cyclin D/p16-Rb signaling pathway is among the most common aberrations found in human cancer (Hanahan D, Weinberg R A. The hallmarks of cancer. Cell 2000; 100: 57-70). Mutations in p16 have been found in >70 different types of tumor cells (as reviewed in Cordon-Cardo, 1995). In the case of ovarian cancer, p16 (also called MTS1 or CDKN2) expression is most commonly altered due to promoter methylation, and less commonly by homozygous deletion or mutation. A recent report indicates that of 249 ovarian cancer patients, 100 (40%) tested positive for p16 promoter methylation (Katsaros D, Cho W, Singal R, Fracchioli S, Rigault De La Longrais I A, Arisio R, et al. Methylation of tumor suppressor gene p16 and prognosis of epithelial ovarian cancer. Gynecol Oncol 2004; 94:685-92). Homozygous deletions of the p16 gene (CDKN2A) were detected in 16/115 (14%) or 8/45 (18%) (Schultz D C, Vanderveer L, Buetow K H, Boente M P, Ozols R F, Hamilton T C, et al. Characterization of chromosome 9 in human ovarian neoplasia identifies frequent genetic imbalance on 9q and rare alterations involving 9p, including CDKN2. Cancer Res 1995; 55:2150-7; Kudoh K, Ichikawa Y, Yoshida S, Hirai M, Kikuchi Y, Nagata I, et al. Inactivation of p16/CDKN2 and p15/MTS2 is associated with prognosis and response to chemotherapy in ovarian cancer. Int J Cancer 2002; 99:579-82), and mutations in 53/673 (8%) of ovarian cancers (www.sanger.ac.uk/genetics/CGP/cosmic). Thus, by these estimates, greater than 60% of ovarian cancers have misregulation of p16.

A novel opportunity to intervene in ovarian and other cancers, including pancreatic where DNA replication is affected due to a decrease in expression of p16 or mutations that affect its activity, is to replace functional p16 protein. In certain embodiments, functional p16 protein is replaced in cells or tissues that are Rb⁺ tumor cells. Functional replacement would thereby inhibit assembly of active cyclin D-CDK4/6 protein entities, and thus inhibit the phosphorylation of the Rb protein. The present disclosure provides an approach for p16 replacement therapy using cell penetration proteins that facilitate delivery of therapeutics into cells. Moreover, the present disclosure provides evidence that, depending on the particular cell penetration protein (e.g., CPM) chosen, delivery is not ubiquitous. Rather, there is a level of specificity and preferential localization to some tissues over others. Without wishing to be bound by theory, this not only facilitates delivery, but may also decrease side effects and decrease the required effective dosage.

Thus, we describe a novel approach for replacement of p16 function through direct delivery of a functional p16 protein, or functional fragment thereof) to tumor cells that are, optionally, Rb⁺ tumor cells by fusion to the protein entity of the disclosure. For example, a protein entity comprising a target-binding region and a CPM can be used to delivery p16 and therefore replace deficient levels of this tumor suppressor due to, for example, promoter methylation or homozygous deletion or mutation.

Importantly, in knock out mouse studies, p16 has been demonstrated to be a haplo-insufficient locus, meaning that cells are sensitive to the levels of p16. This suggests that altering levels through direct delivery of the protein will have meaningful effect on apoptosis induction.

Additionally, as detailed above, functional variants and functional fragments of p16 that, for example, display less conformational flexibility and/or less tendency to aggregate may be delivered as the p16 portion of the fusion protein instead of a native human sequence.

Evaluation of anti-tumor efficacy of a protein entity of the disclosure comprising a p16 tumor suppressor protein, or a functional fragment or variant thereof, as a novel cancer therapeutic can be performed in preclinical cancer models or in in vitro biochemical or cell biological assays of p16 function. Demonstration of the effects of p16 replacement therapy through a fusion with a protein entity can be through evaluation of apoptosis induction, evaluation of the effects on Rb phosphorylation, and effects on the cell cycle. Initially, these effects can be evaluated on human cancer cell lines in vitro, with follow up studies in human tumor xenografts, including explants from human derived tissues, following either systemic or intraperitoneal delivery. Assays may be carried our using, for example, ovarian, pancreatic, or ovarian cancer cell lines and/or xenograft models.

For a human therapeutic intervention, a protein entity of the disclosure would be expected to provide a maximized therapeutic effect while allowing patients to minimize chemotherapy side effects by avoiding drugs that cause excessive toxicity.

Furthermore, intraperitoneal delivery would be expected to maximize the delivery of drug to tumor cells, particularly when treating ovarian cancer, or a primary or metastatic lesion in the abdominal cavity (e.g., liver mets). The ability to administer protein entities of the disclosure, such as fusion proteins, directly to the intraperitoneal cavity will provide for the highest concentrations to be achieved at the tumor site, including the ovaries and fallopian tubes, and sites of typical metastases. As ovarian cancer tends to recur and progress within the abdominal cavity, regional intraperitoneal therapy for ovarian cancer is attractive. Furthermore the opportunity for repeated regional IP delivery by placement of an IP catheter for multiple courses of treatment provides further advantage. In certain embodiments, a protein entity of the disclosure is administered intraperitoneally. In other embodiments, a protein entity of the disclosure is administered intratumorally. Intratumoral administration provides many of the benefits of IP administration in terms of maximizing dose to the tumor and minimizing exposure to healthy tissues. However, systemic administration is also contemplated.

Subpopulations of patients most likely to respond to treatment may be identified for specific intervention. Selection of such patients can be through immunohistochemistry studies for alterations in p16 expression. Thus, a p16 fusion as a therapeutic can taking advantage of personalized therapy. Furthermore, patients can be selected through immunohistochemistry studies for alternations in Rb expression where patients who are Rb competent as more likely to respond to a p16 replacement protein.

As mentioned, recurrence following treatment of ovarian cancer is frequent, and is complicated by the emergence of drug resistance. As CPMs deliver their cargo by entering cells through an endocytic process involving heparan sulphate proteoglycans, typical emergence of drug resistance is unlikely to affect this class of drugs.

Additionally, in early or advanced stages of disease, a p16 therapeutic of the disclosure can be used in novel combination regimens with existing approved therapeutics or new agents, for example combining with CDK4/6 inhibitors or other therapeutics specifically affecting the cell cycle, or tumor cell growth in general.

Given the role of p16 as a tumor suppressor protein, in certain embodiments, protein entities of the disclosure comprise p16 or a functional fragment or functional variant thereof. In other words, the tumor suppressor portion of the protein entity comprises, in certain embodiments, p16 (such as human p16) or a functional fragment or functional variant thereof. Such protein entities may be particularly suitable for in vitro studies of cells deficient in p16 expression and/or function as models of tumorogenesis. Additionally or alternatively, such protein entities may be administered to a subject comprising cells and tissues in which p16 expression and/or function is deficient. Such studies could be used to deliver p16 protein to cells, including cells deficient for or having low expression of p16 and cell that are Rb+. Moreover, such studies could be used to increase p16 expression and/or function in patients in need thereof (e.g., patients having a p16 deficiency—particularly a deficiency associated with a hyperplastic or neoplastic state—including a hyperplastic or neoplastic state where cells have a deficiency in p16 but are Rb+). In certain embodiments, the patient in need thereof has p16 deficiency associated with melanoma, ovarian cancer, pancreatic cancer, cervical cancer, or hepatocellular carcinoma. In certain embodiments, the patient has a p16 deficient cancer that has metastasized to the liver.

The foregoing are merely exemplary of tumor suppressor proteins that can be the cargo region of a protein entity of the disclosure.

Transcription Factors

In certain embodiments, the cargo region comprises a transcription factor. Without being bound by theory, protein entities in which the cargo region is a transcription factor are suitable for replacement strategies in which subjects have a deficiency in the quantity or function of a transcription factor, such as due to mutation, and this deficiency causes (directly or indirectly) some undesirable symptoms or condition.

The protein entity of the disclosure comprising a transcription factor cargo region (e.g., the cargo region comprises a transcription factor) is delivered into cells where it can provide needed activity. Generally, transcription factors function in the nucleus of a cell, and thus, preferably the transcription factor is delivered into the nucleus of a cell. Such deliver may be facilitated by inclusion of an NLS on some portion of the protein entity, or by retaining an endogenous NLS from the selected transcription factor. Of course, it will be understood that the transcription factor may but need not be endogenously expressed only in those tissues.

A transcription factor is a protein that binds to specific nucleic acid sequences, directly or via one or more additional proteins, to modulate transcription. Transcription factors perform this function alone or with other proteins in a protein entity. Transcription factors sometimes function to promote or activate transcription and sometimes to block or repress transcription. Some transcription factors are either activators or repressors, and others can perform either function depending on the context (e.g., promote expression of some targets but repress expression of other targets). The effect of a transcription factor may be binary (e.g., transcription is turned on or off) or a transcription factor may modulate the level, timing, or spatio-temporal regulation of transcription.

A defining feature of transcription factors is that they contain one or more DNA-binding domains (DBDs). DBDs recognize and bind to specific sequences of DNA adjacent to the gene(s) being regulated by the transcription factor. Transcription factors are often classified based on their DBDs which help define the sequences bound, and thus, help define possible target genes.

Generally, transcription factors bind to either enhancer or promoter regions of DNA adjacent to the genes that they regulate. As noted above, depending on the transcription factor, the transcription of the adjacent gene is either up- or down-regulated. Transcription factors use a variety of mechanisms for the regulation of gene expression.

Transcription factors play a key role in many important cellular processes. As such, their misregulation can be deleterious to the subject. Some of the important functions and biological roles transcription factors are involved in include, but are not limited to, mediating differential enhancement of transcription, development, mediating responses to intercellular signals, facilitating the response to the environment, cell cycle control, and pathogenesis. These functions for transcription factors are briefly summarized below.

Some transcription factors differentially regulate the expression of various genes by binding to enhancer regions of DNA adjacent to regulated genes. These transcription factors are critical to making sure that genes are expressed in the right cell at the right time and in the right amount, depending on the changing requirements of the organism.

Many transcription factors are involved in development. In response to various internal or external stimuli, these transcription factors turn on/off the transcription of the appropriate genes, and help mediate processes such as changes in cell morphology, cell fate determination, proliferation, and differentiation.

Some transcription factors also help cells communicate with each other. This is often mediated via signaling cascaded initiated by cell-cell interactions and/or ligand-receptor interactions. Transcription factors are often downstream components of signaling cascades and, help up or down-regulate transcription in response to the signaling cascade.

Not only do transcription factors act downstream of signaling cascades related to biological stimuli but they can also be downstream of signaling cascades involved in environmental stimuli. Examples include heat shock factor (HSF), which upregulates genes necessary for survival at higher temperatures, hypoxia inducible factor (HIF), which upregulates genes necessary for cell survival in low-oxygen environments, and sterol regulatory element binding protein (SREBP), which helps maintain proper lipid levels in the cell.

Transcription factors can also be used to alter gene expression in a host cell to promote pathogenesis. A well studied example of this are the transcription-activator like effectors (TAL effectors) secreted by Xanthomonas bacteria.

The foregoing are exemplary of categories of transcription factors and, in certain embodiments, a member of any one or more of such categories of transcription factors may be used as a cargo region.

Transcription factors are modular in structure and contain the following domains:
DNA-binding domain (DBD)
Trans-activating or Trans-activation domain (TAD)
(optional) Signal sensing domain (SSD).

In certain embodiments, the cargo region is a transcription factor, and the transcription factor is a human protein. In certain embodiments, the cargo region does not include a transcription factor. In certain embodiments, the protein entity does not include a transcription factor.

### (vii) Applications

The present disclosure also provides methods for using protein entities of the disclosure. The protein entities of the present disclosure can be applied in various types of therapeutic, diagnostic or research settings. According to the disclosure, the cell surface target-binding region of the protein entities of the present disclosure may be an antibody, antibody fragment or antibody mimic. The present disclosure provides the cell surface target binding region as part of a protein entity that enhances penetration of the protein entity into cells expressing the cell surface target (e.g., due to the cell penetrating ability of the CPM and the targeting specificity of the target-binding region). The protein entities preferentially enhance cell penetration. The target-binding region may also be a therapeutic agent or diagnostic agent or research agent itself. The protein entity of the disclosure enhances at least one of the following capacities of its target-binding region: cell penetration, endosomal release, endosomal localization, cytosol re-localization, nucleus re-localization, or other intracellular compartment or sub-compartment re-localization. The protein entities of the disclosure may also be complexed (i.e., fused or combined or conjugated) with a cargo region as described above. The protein entity of the disclosure enhances at least one of the following capacities of the cargo region conjugated to the protein entity: cell penetration capacity, endosomal release, endosomal localization, cytosol re-localization, nucleus re-localization, or other intracellular compartment or sub-compartment re-localization. Also contemplated are methods in which an agent (e.g., a protein, peptide, nucleic acid, or small molecule such as a cytotoxic agent) is co-administered or co-delivered (e.g., whether in vitro or in vivo) in trans with the protein entity. In other words, also contemplated are embodiments in which an agent that is not appended to the protein entity is co-administered or delivered.

According to the disclosure, any target binding region may be provided as a protein entity with a CPM and delivered to a subject to target cells that express a cell surface target bound by the target binding region. Given the ability to readily make and test antibodies, antibody-mimics and adhesin molecules, and thus, to generate target binding regions capable of binding to a cell surface target of interest and having a desired activity (e.g., a desired specificity, affinity, and the like), target binding regions to virtually any cell surface target can be readily generated. Such target binding regions may have any suitable configuration (e.g., antibody, antibody fragment, antibody mimic, etc.). The present system may be used in combination with any cell surface target, such as a protein, a polypeptide or peptide, an enzyme, a growth factor, a lipid, a lipoprotein, a glycoprotein, cholesterol, present on the cell surface. Accordingly, the protein entities of the disclosure have numerous applications, including research uses, therapeutic uses, diagnostic uses, imaging uses, and the like, and such uses are applicable over a wide range of targets and disease indications.

### Exemplary Research Uses

Protein entities of the disclosure may be used in research to evaluate protein uptake (e.g., cell penetration or internalization), protein localization, intracellular trafficking, and protein-protein interactions. Moreover, protein entities of the disclosure may be used to evaluate the impact of delivering a protein entity, such as a protein entity appended with a cargo region, into a cell—particularly in a targeted fashion (e.g., a manner dependent on binding of the target binding region to the cell surface target). Additionally, protein entities of the disclosure may be used to evaluate the balance between the features of various target binding regions and that of the CPM, as well as the impact on that balance of appending other modules and/or including SRs. Without being bound by theory, the disclosure demonstrates that targeted cell penetration (e.g., non-ubiquitous penetration that is not limited to a narrow area of local administration) is a balance between the cell penetration activity of the CPM and the cell targeting characteristics (e.g., K_{D}, Kₒₙ, K_{off}, etc.) of the target binding region. If the cell penetration activity of the CPM is too low, then there will be minimal or no charge-enhanced penetration relative to the target binding region alone. If the target binding region has a rapid dissociation constant or “off-rate” from its cell surface receptor, then the CPM may be used to achieve prolonged association with the cell surface, potentially leading to enhanced cell penetration.

The particular applications of the technology will depend upon the target binding region chosen (e.g., what cell surface target does it bind), the CPM, and whether the protein entity is appended to a cargo region. If present, the cargo region may significantly impact the likely applications of the technology. For example, if the protein entity is conjugated to a drug (e.g., a small molecule, such as a cytotoxic agent), the suitable applications and in vitro uses will likely be determined by the nature and function of the drug. For example, conjugates to chemotherapeutics and cytotoxic agents have uses in cancer.

### Exemplary Uses

The protein entities of the disclosure, including entities that are appended with a cargo region, may be administered to subjects, such as for diagnostic, imaging, or therapeutic purposes. In such embodiments, the nature of the cargo region will influence the specific method of use for the protein entity.

By way of example, in certain embodiments, the cargo region is an enzyme and the protein entity when complexed with the enzyme cargo enhances targeted delivery and cell penetration of the enzyme cargo and thus is able to supplement endogenous enzyme expressions.

By way of further example, in certain embodiments, the cargo region is a small organic molecule, such as a cytotoxic or chemotherapeutic agent. Protein entities complexed with such a small organic molecule as a cargo region are suitable for preferential, non-ubiquitous delivery (specific targeting and enhanced penetration) of a cancer therapeutic into cancer cells that overexpressing a surface target (such as breast cancer cells overexpressing Her2 receptors).

By way of further example, in certain embodiments, the cargo region is a tumor suppressor protein. Protein entities complexed with a tumor suppressor protein are suitable for preferential, non-ubiquitous delivery of such tumor suppressor proteins to regulate expression and/or activity of the tumor suppressor protein in cells of specific type. One such tumor suppressor protein is p16.

Any target binding region may be provided in association with a CPM, and delivered to a cell using the inventive system. Given the ability to readily make and test antibodies and antibody-mimics, and thus, to generate target binding region capable of binding to a target and having a desired activity, specificity, and binding kinetics, the present system may be used in combination with virtually any cell surface target to preferentially target a protein entity for penetration into those cells. Accordingly, the protein entities of the disclosure have numerous applications, including research uses, therapeutic uses, diagnostic uses, imaging uses, and the like, and such uses are applicable over a wide range of targets and disease indications.

The following provides specific examples, including examples of specific targets. However, the potential uses of protein entities of the disclosure are not limited to specific target polypeptides or peptides.

By way of example, protein entities of the disclosure can be used to deliver an anti-CD52 antibody into lymphoma cells expressing GPI-anchored proteins (e.g., CD52). By way of another example, protein entities of the disclosure can be used to deliver an anti-HER2 antibody into cancer cells overexpressing HER2 receptors. Protein entities of the disclosure can achieve a preferential, non-ubiquitous delivery (specific targeting and enhanced penetration) of the therapeutic antibodies due to the penetration ability of the CPM and the specific binding ability of the antibody.

In addition, protein entity of the disclosure may be used in research setting to study target expression, presence/absence of target in a disease state, impact of inhibiting or promoting target activity, etc. Protein entities of the disclosure are suitable for these studies in vitro or in vivo.

Further, protein entity of the disclosure have therapeutic uses by enhancing penetration of target binding moieties into cells in humans or animals (including animal models of a disease or condition). Once again, the use of protein entity of the disclosure decrease failure of an target binding moiety due to inability to effectively penetrate cells or due to the inability to effectively penetrate cells at concentrations that are not otherwise toxic to the organism.

Regardless of whether a protein entity of the disclosure is used in a research, diagnostic, prognostic or therapeutic context, the result is that the cargo region is delivered into a cell following contacting the cell with the protein entity (e.g., either contacting a cell in culture or administrated to a subject).

### (viii) Pharmaceutical Compositions

The present disclosure provides protein entities of the disclosure (e.g., a CPM-associated with a target binding region). This section describes exemplary compositions, such as compositions of a protein entity of the disclosure formulated in a pharmaceutically acceptable carrier. Any of the protein entities comprising any of the CPMs and any of the target binding regions described herein may be formulated in accordance with this section of the disclosure.

Thus, in certain aspects, the present disclosure provides compositions, such as pharmaceutical compositions, comprising one or more such protein entities, and one or more pharmaceutically acceptable excipients. Pharmaceutical compositions may optionally include one or more additional therapeutically active substances. In accordance with some embodiments, a method of administering pharmaceutical compositions comprising one or more CPM or one or more protein entities of the disclosure (e.g., a protein entity comprising a CPM or/associated with at least one target binding region) to be delivered to a subject in need thereof is provided. In some embodiments, compositions are administered to humans. For the purposes of the present disclosure, the phrase “active ingredient” generally refers to a target binding region connected with a CPM portion (or portion) to be delivered as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts, as well as suitable or adaptable for research use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects or patients to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100% (w/w) active ingredient.

Pharmaceutical formulations may additionally include a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, an active ingredient is mixed with at least one inert, pharmaceutically acceptable excipient. In the case of capsules, tablets and pills, the dosage form may comprise buffering agents.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

In certain embodiments, protein entities of the disclosure and compositions of the disclosure, including pharmaceutical preparations, are non-pyrogenic. In other words, in certain embodiments, the compositions are substantially pyrogen free. In one embodiment, the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### (ix) Administration

The present disclosure provides compositions and methods for binding a cell surface target and enhancing internalization of a protein entity comprising a target binding region that binds the cell surface target and a CPM. The protein entity comprising a target binding region and a CPM is administered into a subject (e.g., a human or animal), thereby promoting delivery of the target binding region (and the protein entity, including any additional regions or modules appended thereto) into the cell. Moreover, the protein entities can be used on cells in culture to study function of the protein entities, kinetics of binding and internalization, protein-protein interaction, co-administration of agents, and the like. In such cases, administration includes contacting cells in vitro, such as by adding a protein entity to a culture of cells.

The present disclosure provides methods comprising administering CPM/target binding region protein entities to a subject in need thereof. The disclosure contemplates that any of the protein entities of the disclosure (e.g., protein entities including a CPM and a target binding region) may be administered, such as described herein. Protein entities of the disclosure, including as pharmaceutical compositions, may be administered or otherwise used for research, diagnostic, imaging, prognostic, or therapeutic purposes, and may be used or administered using any amount and any route of administration effective for preventing, treating, diagnosing, researching or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the disclosure are typically formulated in dosage unit form for ease of administration and uniformity of dosage. It will be understood, however, that the total daily usage of the compositions of the present disclosure will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Protein entities of the disclosure may be administered by any route and may be formulated in a manner suitable for the selected route of administration or in vitro application. In some embodiments, protein entities of the disclosure, and/or pharmaceutical, prophylactic, diagnostic, or imaging compositions thereof, are administered by one or more of a variety of routes, including oral, intravenous, intramuscular, intra-arterial, intramedullary, intrathecal, subcutaneous, intraventricular, transdermal, intradermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. Other devices suitable for administration include, e.g., microneedles, intradermal specific needles, Foley's catheters (e.g., for bladder instillation), and pumps, e.g., for continuous release.

In some embodiments, protein entities of the disclosure (e.g., including protein entities that further comprise a cargo region appended thereto), and/or pharmaceutical, prophylactic, diagnostic, research or imaging compositions thereof, are administered by systemic intravenous injection. In specific embodiments, protein entities of the disclosure and/or pharmaceutical, prophylactic, research, diagnostic, or imaging compositions thereof may be administered intravenously and/or orally. In specific embodiments, protein entities of the disclosure, and/or pharmaceutical, prophylactic, research diagnostic, or imaging compositions thereof, may be administered in a way which allows the protein entity to cross the blood-brain barrier, vascular barrier, or other epithelial barrier.

Protein entities of the disclosure comprising at least one target binding region and a CPM may be used in combination with one or more other therapeutic, prophylactic, diagnostic, research or imaging agents. By “in combination with,” it is not intended to imply that the agents must be administered at the same time and/or formulated for delivery together, although these methods of delivery are within the scope of the disclosure. Compositions of the disclosure can be administered concurrently with, prior to, or subsequent to, one or more other desired therapeutics, other reagents or medical procedures. In general, each agent will be administered at a dose and/or on a time schedule determined for that agent. In some embodiments, the disclosure encompasses the delivery of pharmaceutical, prophylactic, diagnostic, research or imaging compositions in combination with agents that improve their bioavailability, reduce and/or modify their metabolism, inhibit their excretion, and/or modify their distribution within the body. In certain embodiments where an additional agent is co-administered with a protein entity of the disclosure, the protein entity and the other agent are co-administered at approximately the same time or within a period less than or equal to the half-life of one or both agents. It should be understood that an agent may be a protein, nucleic acid, or small molecule (e.g., drug) agent. In certain embodiments, the protein entity comprises an agent (e.g., a cargo region) appended thereto and an additional agent (which may be the same or different) is also co-administered in trans.

It will further be appreciated that therapeutic, prophylactic, diagnostic, research or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the disclosure may be administered concurrently with a chemotherapeutic agent), or they may achieve different effects (e.g., control of any adverse effects).

(x) Kits

The disclosure provides a variety of kits (or pharmaceutical packages) for conveniently and/or effectively providing protein entities of the disclosure (including fusion protein) and/or for carrying out methods of the present disclosure. Typically kits will comprise sufficient amounts and/or numbers of components to allow a user to perform multiple treatments of a subject(s) and/or to perform multiple experiments for desired uses (e.g., laboratory or diagnostic uses). Alternatively, a kit may be designed and intended for a single use. Components of a kit may be disposable or reusable.

In some embodiments, kits include one or more of (i) a CPM as described herein and a target binding region to be delivered; and (ii) instructions (or labels) for forming protein entities comprising the CPM associated with the target binding region (e.g., with at least one target binding region). Optionally, such kits may further include instructions for using the protein entity in a research, diagnostic or therapeutic setting.

In some embodiments, a kit includes one or more of (i) a CPM portion (or portion) as described herein and a target binding region to be delivered or a protein entity of such CPM associated with such target binding region; (ii) at least one pharmaceutically acceptable excipient; (iii) a syringe, needle, applicator, etc. for administration of a pharmaceutical, prophylactic, diagnostic, or imaging composition to a subject; and (iv) instructions and/or a label for preparing the pharmaceutical composition and/or for administration of the composition to the subject. Optionally, the kit may include one or more other agents, including a research reagent or a therapeutic agent, provided in a separate container from the protein entity. When a kit includes one or more additional agents, optionally, instructions and/or a label for co-administration (at the same or differing times) may be provided.

In some embodiments, a kit includes one or more of (i) a pharmaceutical composition comprising a protein entity of the disclosure (e.g., a CPM as described herein associated with a target binding region to be delivered); (ii) a syringe, needle, applicator, etc. for administration of the pharmaceutical, prophylactic, diagnostic, or imaging composition to a subject; and (iii) instructions and/or a label for administration of the pharmaceutical, prophylactic, diagnostic, or imaging composition to the subject. Optionally, the kit need not include the syringe, needle, or applicator, but instead provides the composition in a vial, tube or other container suitable for long or short term storage until use.

In some embodiments, a kit includes one or more components useful for modifying proteins of interest, such as by supercharging the protein (e.g., charge engineering the protein), to produce a CPM. These kits typically include all or most of the reagents needed. In certain embodiments, such a kit includes computer software to aid a researcher in designing the engineered or otherwise modified CPM in accordance with the disclosure. In certain embodiments, such a kit includes reagents necessary for performing site-directed mutagenesis.

In some embodiments, a kit may include additional components or reagents. For example, a kit may include buffers, reagents, primers, oligonucleotides, nucleotides, enzymes, buffers, cells, media, plates, tubes, instructions, vectors, etc. The additional reagents are suitable for the particular use, such as research, therapeutic, diagnostic, or imaging use.

In some embodiments, a kit comprises two or more containers. In certain embodiments, a kit may include one or more first containers which comprise a CPM, and optionally, at least one target binding region molecule to be delivered, or a protein entity comprising a CPM and at least one target binding region to be delivered for diagnosing or prognosing a disease, disorder or condition or for research use; and the kit also includes one or more second containers which comprise one or more other prophylactic or therapeutic agents useful for the prevention, management or treatment of the same disease, disorder or condition, or useful for the same research application.

In some embodiments, a kit includes a number of unit dosages of a pharmaceutical, prophylactic, diagnostic, or imaging composition comprising a protein entity of the disclosure or comprising a CPM, and optionally, at least one target binding region to be delivered. In some embodiments, the unit dosage form is suitable for intravenous, intramuscular, intranasal, oral, topical or subcutaneous delivery. Thus, the disclosure herein encompasses solutions, preferably sterile solutions, suitable for each delivery route. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical, prophylactic, diagnostic, or imaging compositions, may be included to provide a kit in which a dosage is taken every day.

In some embodiments, the kit may further include a device suitable for administering the composition according to a specific route of administration or for practicing a screening assay.

Kits may include one or more vessels or containers so that certain of the individual components or reagents may be separately housed. Exemplary containers include, but are not limited to, vials, bottles, pre-filled syringes, IV bags, blister packs (comprising one or more pills). A kit may include a means for enclosing individual containers in relatively close confinement for commercial sale (e.g., a plastic box in which instructions, packaging materials such as styrofoam, etc., may be enclosed). Kit contents can be packaged for convenient use in a laboratory.

In the case of kits sold for laboratory and/or diagnostic use, the kit may optionally contain a notice indicating appropriate use, safety considerations, and any limitations on use. Moreover, in the case of kits sold for laboratory and/or diagnostic use, the kit may optionally comprise one or more other reagents, such as positive or negative control reagents, useful for the particular diagnostic or laboratory use.

In the case of kits sold for therapeutic and/or diagnostic use, a kit may also contain a notice in the form prescribed by a governmental agency regulating the manufacture, use or sale of pharmaceuticals or biological products, which notice reflects (a) approval by the agency of manufacture, use or sale for human administration, (b) directions for use, or both.

These and other aspects of the present disclosure will be further appreciated upon consideration of the following Examples, which are intended to illustrate certain particular embodiments of the disclosure but are not intended to limit its scope, as defined by the claims.

### EXEMPLIFICATION

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function. As such, it will be readily apparent that any of the disclosed specific constructs and experimental plan can be substituted within the scope of the present disclosure.

### Example 1

### Production of Charged Proteins Fused to a Single Chain Antibody Against her2

A series of charged GFP proteins and GFP-C6.5 fusion proteins were designed and produced. C6.5 is a single chain variable fragment (scFv; an example of an antibody fragment or antigen binding fragment) that binds to the HER2 receptor (a cell surface target).

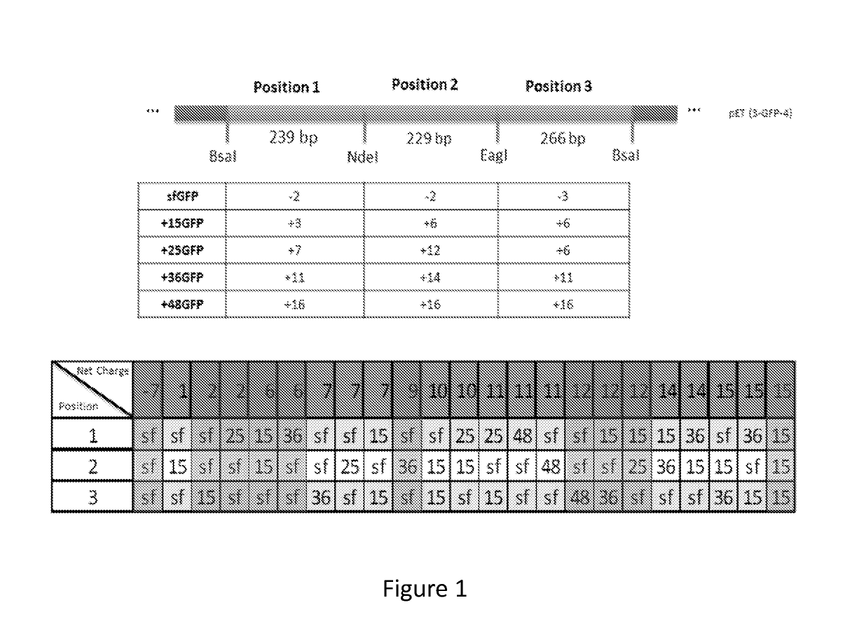

Design of Charge Series: a GFP charge series was designed with charges ranging from +2 to +12. To construct the charge series, the GFP charge variant sequences were split into three parts. These charge variants included sf-(superfolder), +15GFP, +25 GFP, +36GFP, and +48GFP. Three fragments from different variants were combined to obtain a unique GFP charge series (see FIG. 1). Table 5 lists the naming convention for the GFP charge series. In Table 5, the three fragments from the original charge variants used to construct each member of the series with an epitope tag (e.g., a His6 and/or a Myc tag at the either the C-terminus or the N-terminus) are listed under the Sequence column.

Table 5: Naming Convention for GFP Charge Series

TABLE 5
Naming convention for GFP charge series

| GFP Charge | Sequence | Letter | Name |
|---|---|---|---|
| +2 | sf-sf-15 | A | +2GFPa |
| +2 | 25-sf-sf | B | +2GFPb |
| +6 | 15-15-sf | a | +6GFPa |
| +6 | 36-sf-sf | b | +6GFPb |
| +9 | sf-36-sf | — | +9GFP |
| +12 | 15-25-sf | a | +12GFPa |
| +12 | 15-sf-36 | b | +12GFPb |
| +12 | sf-sf-48 | c | +12GFPc |

Construct Design: Constructs produced with the GFP charge variants (GFP_{cv}) included sf, +2-+12 from the charge series, and +15GFP. For each GFP_{cv}, two constructs were made: GFP_{cv}-His₆ and GFP_{cv}-(S₄G)₆-C6.5-His₆. Two constructs with scFv alone were also produced: C6.5-(S₄G)₆-His₆ and His₆-C6.5. We note that the fusion proteins of a CPM and a target-binding region depicted in these examples and used in these experiments included a spacer region (specifically, a spacer region comprising serine and glycine residues) interconnecting the CPM region and the target-binding region. For ease, when referring to the fusion proteins in the remainder of the example, the spacer region is typically not expressing referred to.

Protein Production: All the proteins were produced in the same manner. The expression and purification processes for +9GFP and +12GFPa-C6.5 (which also includes a spacer region) were described herein as examples. The pJExpress416 expression vector containing the coding sequences for +12GFPa-C6.5 or +9GFP alone was transformed into either the SHuffle T7 lysY (NEB) or BL21(DE3) (Life Technologies) strains of E. coli cells, respectively. SHuffle T7 lysY cells were grown at 30° C. and BL21(DE3) cells were grown at 37° C. with shaking at 350 rpm. The cells were grown to a density between 1.1 and 2.0 (as measured by A₆₀₀) in 150 mL Cinnabar media (Teknova) containing 50 μg/mL kanamycin, and 0.005% antifoam (Teknova), induced with 0.5 mM IPTG and incubated at 18° C. with shaking at 350 rpm for 18 hours. Cells were harvested by centrifugation at 6,000×g for ten minutes.

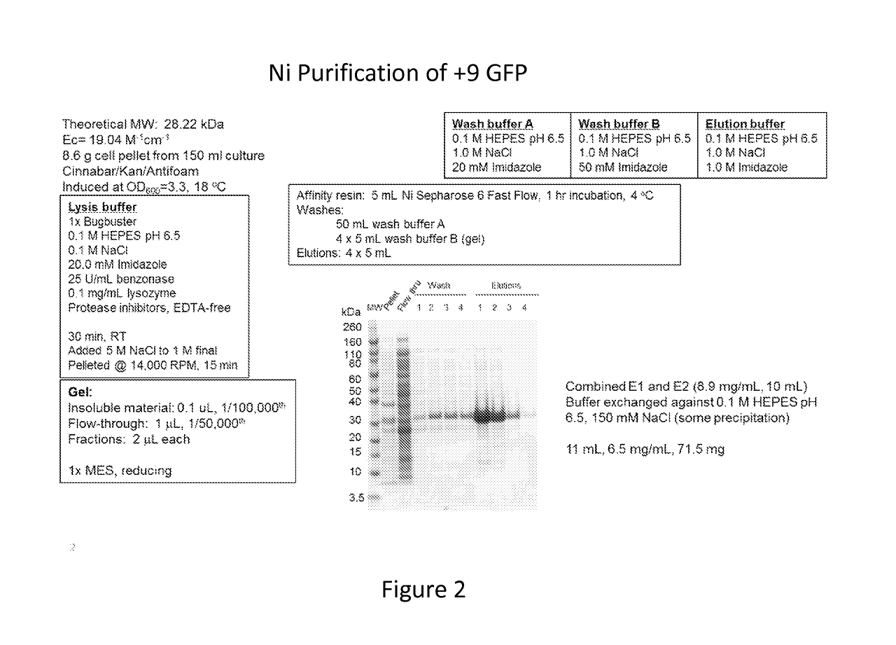

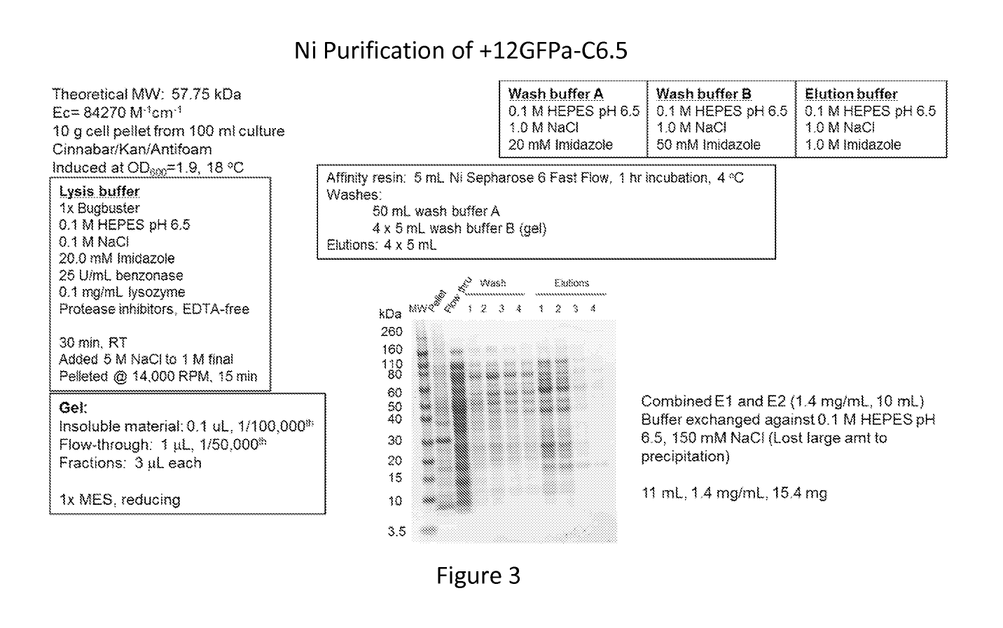

The resulting cell pellet was lysed in lysis buffer (1× Bugbuster, Novagen, 0.1 M HEPES pH 6.5, 0.1 M NaCl, 20 mM imidazole, 25 U/mL benzonase, 0.1 mg/mL lysozyme, and protease inhibitors, complete EDTA free protease inhibitor cocktail tablets, Roche) and the NaCl concentration was subsequently brought to 1.0 M, the lysate was clarified by centrifugation at 20,000×g for ten minutes, and the supernatant was applied to Ni sepharose 6 fast flow resin (GE Healthcare). The bound resin was washed with 10 column volumes (cv) wash buffer A (0.1 M HEPES pH 6.5, 1 M NaCl, 20 mM imidazole), followed by 4×1 cv wash buffer B (A+50 mM imidazole), and eluted with 4×1 cv elution buffer (A+1 M imidazole). Aliquots of representative fractions were applied to 4-12% polyacrylamide gel and visualized with Instant Blue coomassie stain (FIGS. 2 and 3.

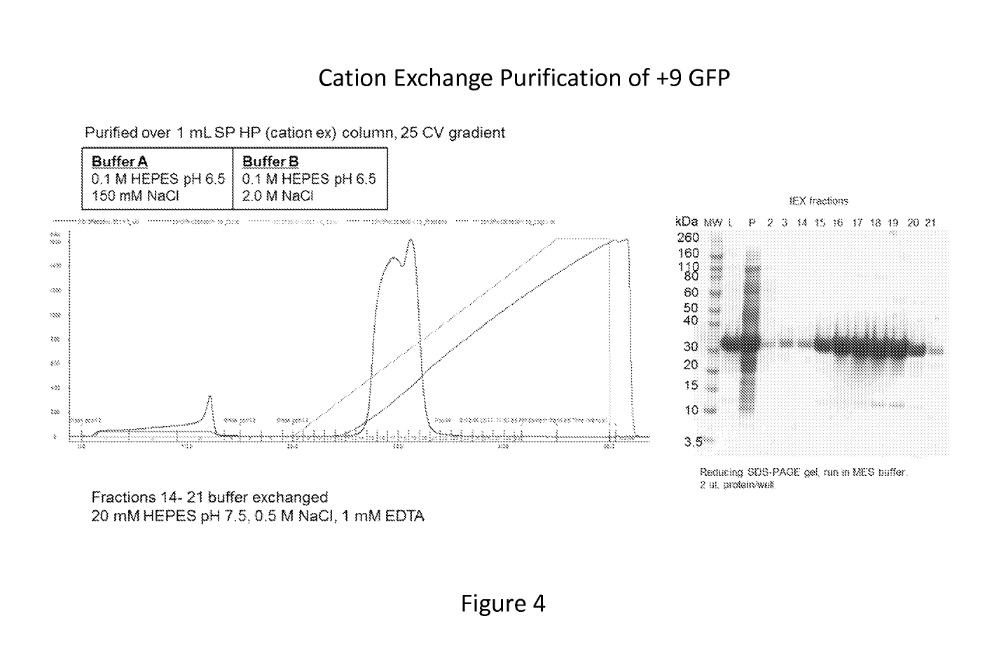

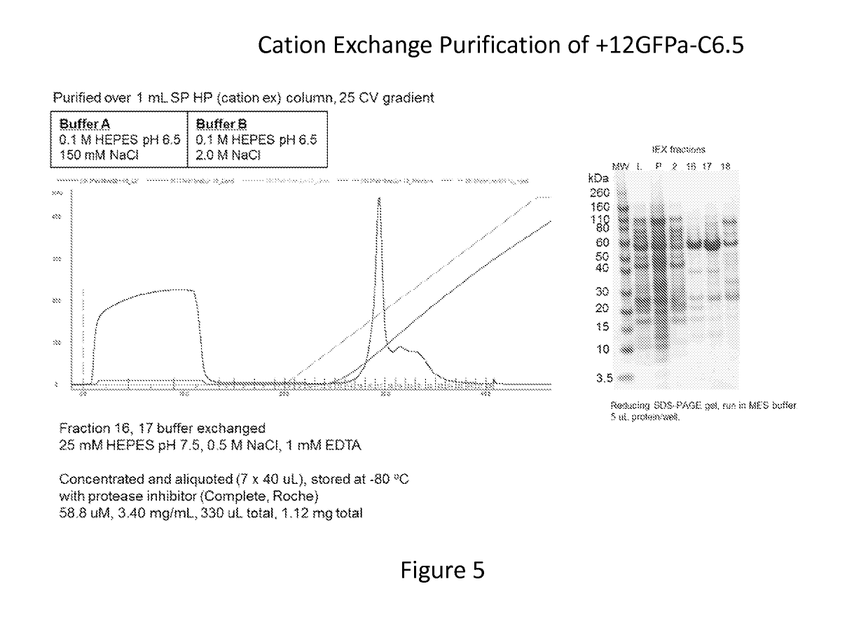

The protein solution was buffer exchanged against 0.1 M HEPES pH 6.5, 150 mM. The protein was centrifuged at 3,500×g for 10 min to remove precipitated protein. The protein was purified by cation exchange chromatography on a HiPrep SP HP 1 mL column (GE Healthcare). The protein was eluted with a gradient of NaCl from 150 mM to 2.0 M over 25 cv (FIGS. 4 and 5).

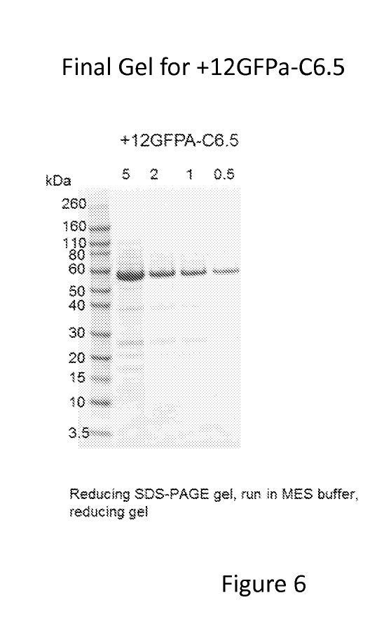

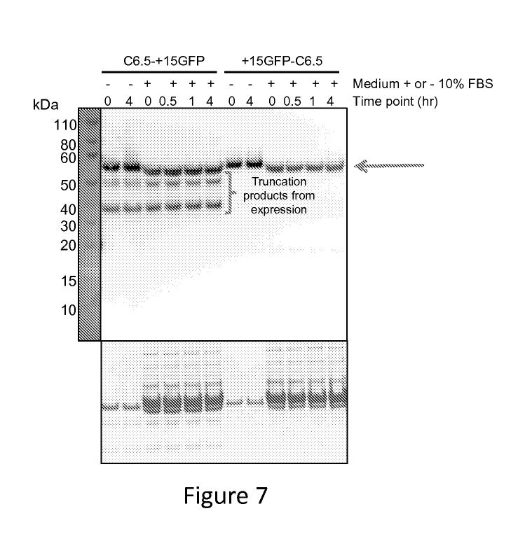

Positive fractions from the cation exchange chromatography were pooled and buffer exchanged against 20 mM HEPES, pH 7.5, 0.5 M NaCl, 1 mM EDTA, and protease inhibitor (only for fusion proteins). If necessary, the protein was concentrated in a 10,000 MWCO Amicon concentrator (Millipore). The final protein product was stored at −80° C. A summary of the purification of +9GFP is as follows: 1) ˜9 g cell paste was produced per 0.15 L of culture; 2) the Ni column yielded 70 mg protein per 0.15 L culture; 3) subsequently, the cation exchange column yielded 58 mg protein; 4) the protein was stored at −80° C. in 20 mM HEPES, pH 7.5, 0.5 M NaCl, 1 mM EDTA; and 5) the final protein was greater than 99% pure. A summary of the purification of +12GFPa-C6.5 is as follows and a gel analysis of the final product is shown in FIG. 6: 1) ˜10 g cell paste was produced per 0.15 L of culture for both; 2) the Ni column yielded 15.4 mg protein per 0.15 L culture; 2) subsequently, the cation exchange column yielded 1.1 mg; 3) the protein was stored at −80° C. in 20 mM HEPES, pH 7.5, 0.5 M NaCl, 1 mM EDTA, and protease inhibitor; and 4) the final protein was 90% pure.

### Example 2

### Serum Stability of Charged Proteins Fused to a Single Chain Antibody Against Her2

Sample preparation: two fusion proteins, i.e., +15GFP-(S₄G)₆-C6.5-His₆ and C6.5-(S₄G)₆-+15GFP-His₆, were evaluated for their stability in 10% fetal bovine serum (FBS) and McCoy's 5A Medium (Gibco, Life Technologies). Proteins were diluted to a final concentration of 1 μM, in 150 μL, in medium or medium containing 10% FBS for each time point (medium only at 0 and 4 hour; medium plus serum at 0, 0.5, 1, and 4 hours). Samples were incubated at 37° C. Samples were quenched with an equal volume (150 μL) of 2× reducing SDS-page sample buffer (Novex, Life Technologies) and stored on ice.

Results: These fusion proteins, in both orientation, were analyzed for serum stability by western blot and both were stable for a minimum of four hours. The results of this Example show that fusion proteins (an example of a protein entity of the disclosure) comprising charged GFP (as the CPM region) and C6.5 scFv (as the target binding region) are stable in 10% serum for at least 4 hours.

### Example 3

### Charged Proteins Fused to a Single Chain Antibody Against Her2 Retains Appropriate Binding Function

In this Example, protein entities comprising various GFP regions from the charged series were fused to C6.5, a scFv that specifically binds Her2. Surface plasmon resonance (SPR) assays were run on a Biacore 3000 to determine the binding kinetics of five C6.5 fusion proteins to the extracellular domain of Her2. The running buffer used for immobilization and kinetic assays was HBS-EP (10 mM HEPES pH 7.4, 150 mM NaCl, 0.005% w/v Surfactant P20, GE Healthcare).

Immobilization: Anti-human IgG (Fc) antibody was directly coupled to a CM5 sensor chip (using the amine coupling and human antibody capture kits from GE Healthcare). The chip surface was activated by injecting a 1:1 (v/v) mixture of 0.5 M EDC and 0.1 M NHS for 7 minutes at 10 μL/minute. The antibody was diluted to 25 μg/mL in 10 mM sodium acetate pH 5.0 and injected at 10 μL/min for 7 minutes. The chip surface was blocked with 1 M ethanolamine hydrochloride-NaOH pH 8.5 for 7 minutes at 10 μL/min

Kinetic Assays: The binding kinetics of each fusion protein for Her2 was determined by generating sensograms via multi-cycle analysis. The ligand, recombinant human ErbB2 Fc chimera (Her2 extracellular domain, R&D Systems), was dissolved in PBS at 100 μg/mL. The ligand was further diluted to 1 μg/mL in HBS-EP running buffer. The ligand was captured by injection over flow cell 2 for 6 minutes at 1 μL/min to obtain a response of approximately 300 RU. The analytes, C6.5 containing fusion proteins (see Table 6), were diluted in running buffer at concentrations of 50, 16.7, 5.6, 1.85, and 0.62 nM and were injected over flow cell 1 and 2 for 1 minute at 30 μL/min. Dissociation was monitored for 5 minutes. Buffer blanks were run in duplicate, as was a single concentration of the fusion protein. After injection and dissociation of each analyte, the chip was regenerated by injection of 3M MgCl₂ for 30 seconds at 30 μL/min Flow cell 1 had no ligand captured and was used as a reference. Data were fitted to a 1:1 binding model to obtain the dissociation equilibrium constant, K_{D}.

Results: The binding kinetics of five C6.5 fusion proteins were analyzed by SPR. See Table 6. The C6.5 constructs without GFP and C6.5-sfGFP construct had similar dissociation constants, all in the low nM range. The two fusion proteins that contained both a CPM region (in this case, +15GFP) and C6.5 had lower dissociation constants, both in the pM range. These results indicate that fusion of the charged CPM, in this case a CPM with a net theoretical charge of +15, to either termini of this target-binding region (C6.5; an scFv that binds specifically to Her2) has no negative effect on C6.5 binding to its receptor, Her-2.

TABLE 6
Dissociation constants of C6.5 fusion proteins
determined by multi-cycle kinetics

| C6.5 fusion protein | K_{D} (nM) |
|---|---|
| C6.5-(S₄G)₆-His₆ | 2.3 |
| His₆-C6.5 | 1.6 |
| +15GFP-(S₄G)₆-C6.5-His₆ | 0.73 |
| C6.5-(S₄G)₆-+15GFP-His₆ | 0.19 |
| C6.5-(S₄G)₆-sfGFP-His₆ | 1.1 |

### Example 4

### Charged Proteins Fused to a Binding Domain Enhance Internalization of the Binding Domain on Cells Expressing the Target of the Binding Domain

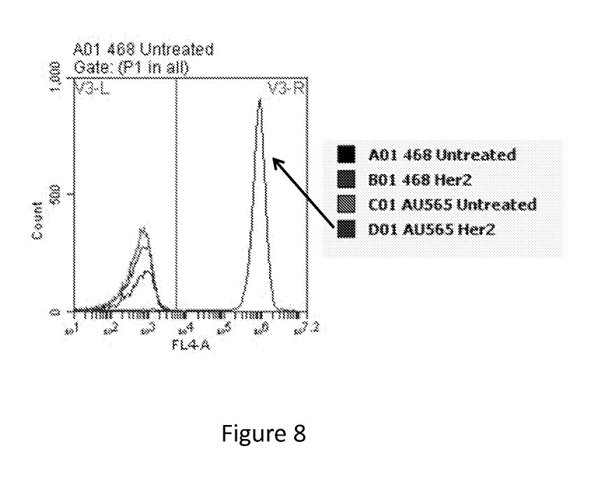

Materials and methods: MDA-MB-468 and AU565 cells were used in this Example. These two types of cells express different levels of Her2 protein (FIG. 8). Her2 protein was detected on MDA-MB-468 and AU565 cells using a commercial antibody against Her2. MDA-MB-468 cells express very low levels of Her2 (referred to as Her2^{Low}) while AU565 cells express high levels of Her2 (referred to as Her2^{high}).

100,000 of each of AU565 (Her2^{high}) and MDA-MB-468 (Her2^{Low}) cells were plated in each well of 12-well plate in growth media overnight. The media were replaced with serum free media containing 1 μM of a protein listed below, and incubated for 2 hours. Cells were washed 3×PBS, trypsinized, fixed with 4% PFA, washed with PBS and then analyzed by flow cytometry with detection of GFP. The following fusion proteins were tested in this Example:
sfGFP-(S₄G)₆-C6.5-His₆
sfGFP-His₆
+6GFPa-(S₄G)₆-C6.5-His₆
+6GFPa-His₆
+9GFP-(S₄G)₆-C6.5-His₆
+9GFP-His₆
+15GFP-(S₄G)₆-C6.5-His₆
+15GFP-His₆
+36GFP-His₆

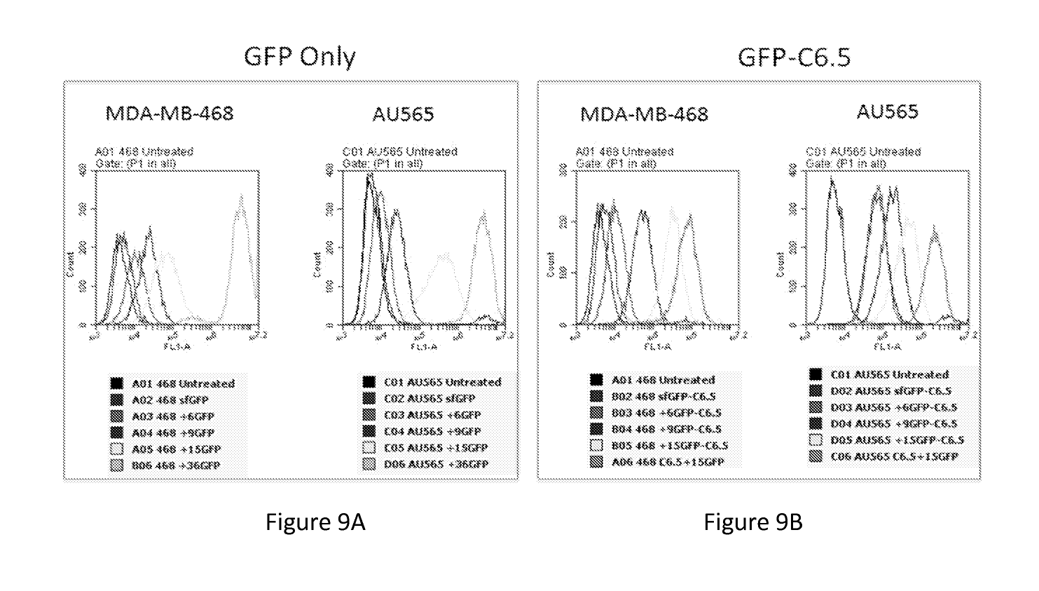

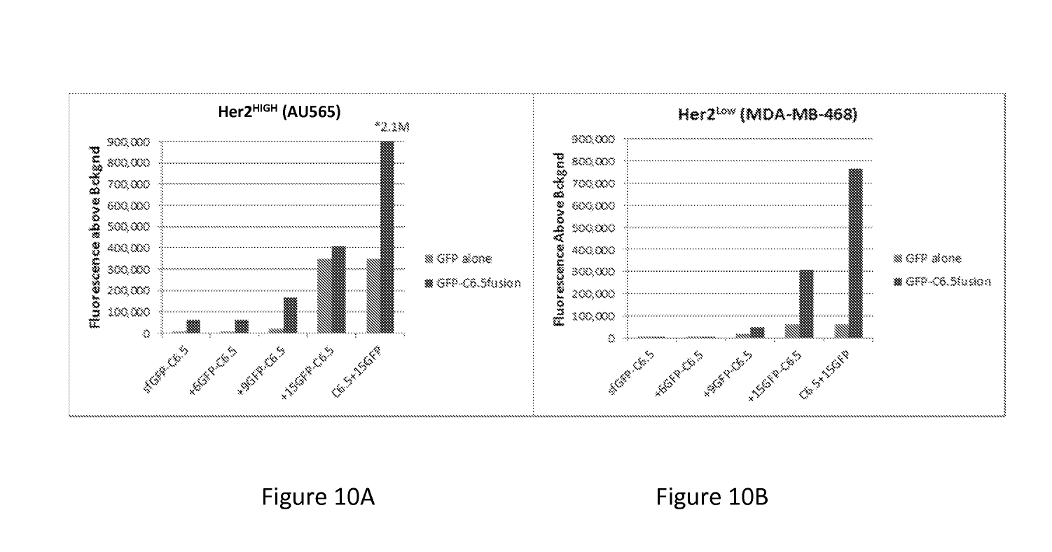

Results: Flow cytometry analysis is indicative of the amount of protein internalized into the cells. FIGS. 9A and 9B show the flow cytometry data obtained for different tested samples at various conditions. The median fluorescence values obtained from the flow cytometry peak minus the median fluorescence values of untreated cells (background fluorescence) are shown in FIGS. 10A and 10B. See also Table 7. The first column indicates identifies the GFP-component of the construct used for the particular sample treatment. The second and third columns represents fluorescence in MDA-MB-468 cells following treatment with each of the GFP proteins alone (second column; examples of use of CPMs alone) or with each of the GFP-C6.5 fusion proteins (third column; examples of fusion proteins comprising a target-binding region and a CPM region). The fourth and fifth column fluorescence in AU565 cells following treatment with each of the GFP proteins alone (fourth column; examples of use of CPMs alone) or with each of the GFP-C6.5 fusion proteins (fifth column; examples of fusion proteins comprising a target-binding region and a CPM region).

TABLE 7

| | MDA-MB-468 (Her2^{Low}) | | AU565 (Her2^{high}) | |
|---|---|---|---|---|
| | GFP alone | GFP-C6.5 | GFP alone | GFP-C6.5 |
| Untreated | 4,064 | 4,064 | 5,713 | 5,713 |
| sfGFP | 5,115 | 5,632 | 5,896 | 69,696 |
| +6GFPa | 10,500 | 10,383 | 9,410 | 68,963 |
| +9GFP | 22,842 | 51,296 | 24,550 | 171,711 |
| +15GFP | 65,344 | 313,629 | 353,626 | 413,838 |
| +36GFP | 5,807,366 | | 4,351,574 | |
| C6.5 + 15GFP | | 767,627 | | 2,170,916 |

sfGFP-C6.5 generated a 12-fold higher signal than sfGFP alone due to binding and internalization of C6.5 (FIG. 10A). There was no such increase in signal when sfGFP-C6.5 was applied to Her2^{Low} cells compared to sfGFP alone (FIG. 10B), and these levels were within 20% of background cell fluorescence as determined from an untreated cell sample. The results indicate that C6.5 is capable of binding to HER2 on Her2^{high} cells when fused with a GFP protein.

These results also indicate that the addition of charge improves the internalization of C6.5. In comparing the +9GFP-C6.5 to the sfGFP-C6.5, the fluorescence is higher by 2.5-fold for +9GFP-C6.5 on the Her2^{high} cells. This boost in internalization appears to be C6.5 dependent as the signal from +9GFP alone on Her2^{high} cells is 3-fold lower than sfGFP-C6.5. Furthermore, a threshold of charge may be needed to see an effect. For example, +6GFP-C6.5 on Her2high cells generated the same signal as sfGFP-C6.5 under these experiment conditions. This suggests that a +6 charge may not be enough charge to enhance internalization under these experimental conditions and/or using a target-binding region of this affinity. Too much charge, however, may overwhelm the binding characteristics of the target-binding region, thus leading to cell internalization independent of target binding. These results indicate that the characteristics of the target-binding region and the CPM can be selected to retain binding of the target-binding region to its cell surface target while still enhancing internalization.

Orientation of the regions of the construct may also influence cell penetration and the extent to which cell internalization is a function of target binding. In fact, the C6.5-+15GFP generated 5-fold higher internalization than +15GFP-C6.5 FIGS. 10A and 10B). These data indicate that +15GFP alone is only 16% of the C6.5-+15GFP signal. As described in Example 3, the Kd value of C6.5-+15GFP is 0.19 nM while the Kd value of +15GFP-C6.5 is 0.73 nM. Given the differing dissociation constants and differing internalization data, these results highlight the balance between the function of the target-binding region and that of the CPM.

Binding and internalization of the proteins increased with charge (FIG. 10B). Furthermore, the GFP-C6.5 proteins had higher internalization than the GFP proteins alone for higher charge GFPs, e.g., the +9GFP and +15GFP. This increase in internalization is more pronounced with +15 than with +9. These results indicate that for cells with low receptor numbers for a target-binding region, more charge may be needed to enhance internalization compared to cells with high receptor numbers. For an in vivo situation where there are many cell types potentially with differential expression of receptors that are being targeted by a target-binding region, the least charge to still see a desirable increase in internalization may be a preferred approach.

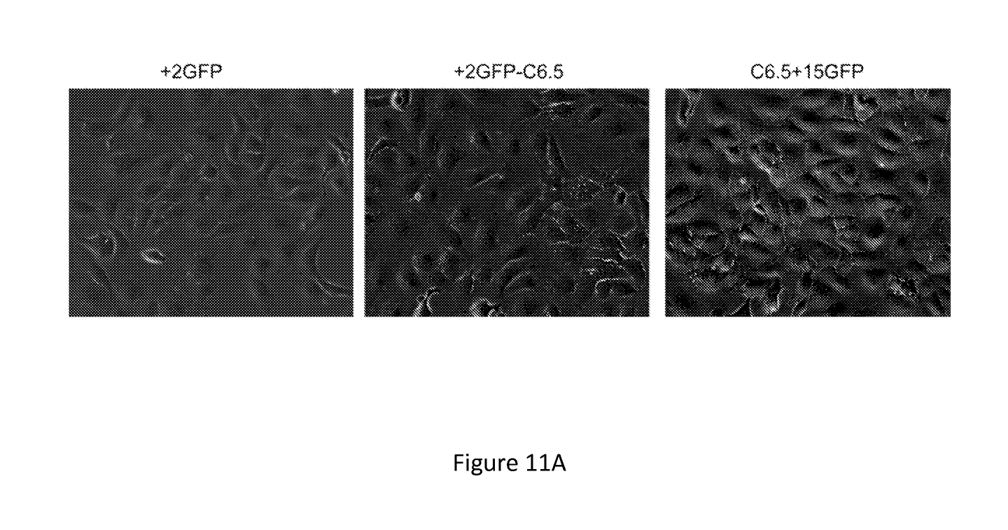

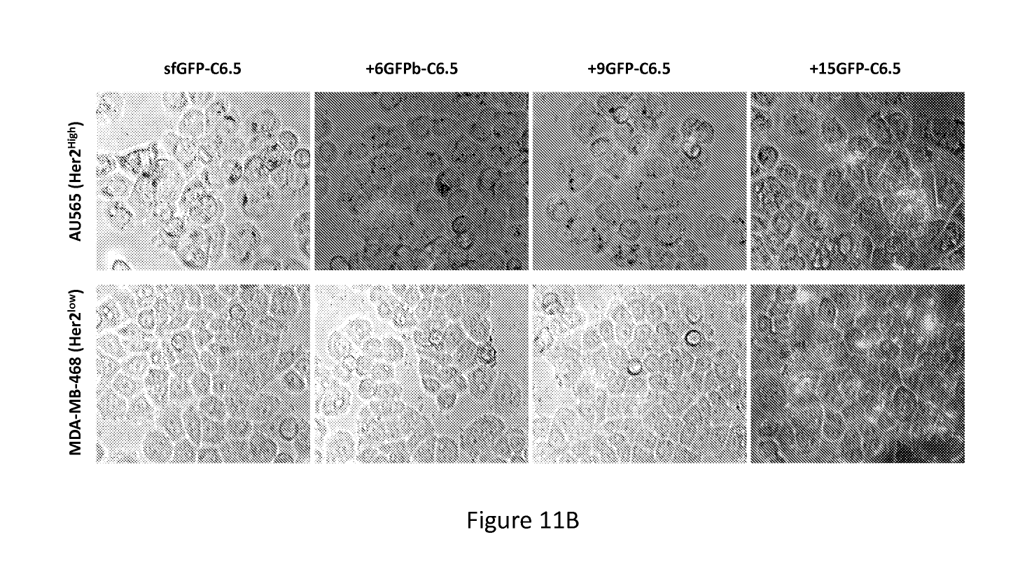

In addition, SKOV-3 cells (Her2^{high}) were treated with 1 μM of proteins for 1 hour, and then images were taken to assess cellular uptake of GFP proteins by fluorescence microscopy (FIG. 11A). The minimum charged +2GFP protein did not bind to SKOV-3 cells significantly. The +2GFP-C6.5 bound to SKOV-3 cells through Her2 but did not internalize in the cells, which was consistent with the mostly cell surface staining. In contrast, the higher charged C6.5+15GFP protein was internalized efficiently in the cells.

### Example 5

### Fusion Proteins Comprising a Target-Binding Region and a CPM Retain Cell-Receptor Specific Binding and have Enhanced Internalization in Mixed Cell Populations

Materials and methods: 100,000 of each of AU565 (Her2^{high}) and MDA-MB-468 (Her2^{Low}) cells were plated in each well of 12-well plate in growth media overnight. The media were replaced with serum free media containing indicated concentrations of protein listed below and incubated for 2 h. Cells were washed 3×PBS, trypsinized, fixed with 4% PFA, stained with Her2 Ab-APC for 0.5 hour, washed with PBS and then analyzed by flow cytometry with detection of GFP. The following proteins were tested in a first set of experiments:
+6GFPa-(S₄G)₆-C6.5-His₆
+6GFPa-His₆
+9GFP-(S₄G)₆-C6.5-His₆
+9GFP-His₆
+15GFP-(S₄G)₆-C6.5-His₆
+15GFP-His₆
C6.5-(S₄G)₆-+15GFP-His₆

The following proteins were tested in a second set of experiments:
sfGFP-(S₄G)₆-C6.5-His₆
sfGFP-His₆
+6GFPb-(S₄G)₆-C6.5-His₆
+6GFPb-His₆
+12GFPa-(S₄G)₆-C6.5-His₆
+12GFPa-His₆
+12GFPc-(S₄G)₆-C6.5-His₆
+12GFPc-His₆

The following proteins are tested in a third set of experiments:
His₆-C6.5-(S₄G)₆-+sfGFP
His₆-C6.5-(S₄G)₆-+6GFPa
His₆-C6.5-(S₄G)₆-+6GFPb
His₆-C6.5-(S₄G)₆-+9GFP
His₆-C6.5-(S₄G)₆-+12GFPa
His₆-C6.5-(S₄G)₆-+12GFPb
His₆-C6.5-(S₄G)₆-+12GFPc
His₆-C6.5-(S₄G)₆-+15GFP

The tested proteins of the first and second sets of experiments were applied to the mixed cell population for two hours.

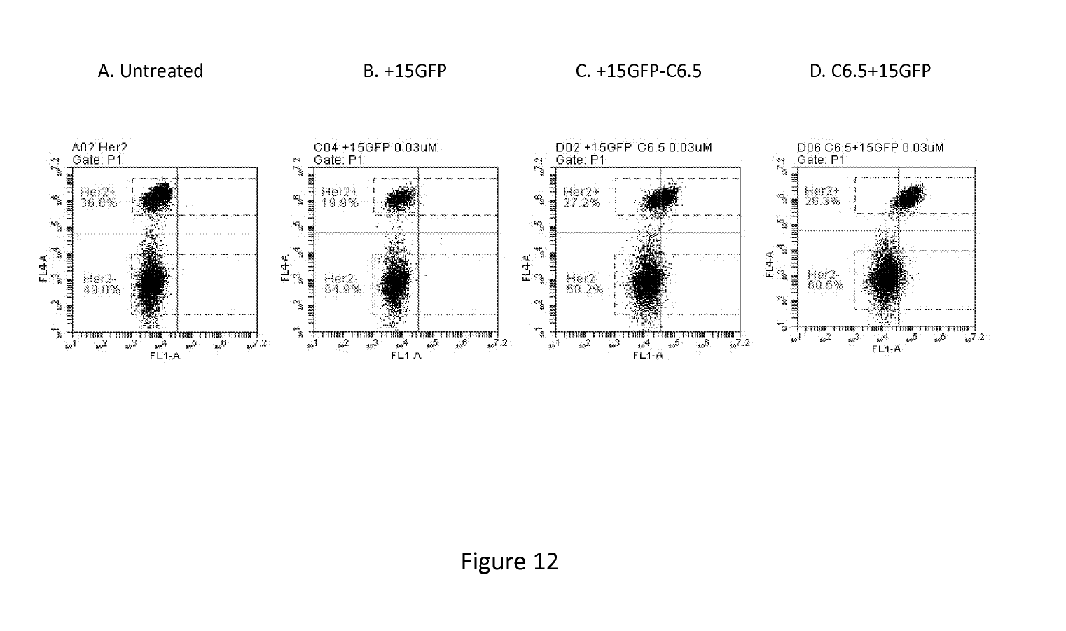

Results: as shown in FIGS. 12A-12D, cellular uptake in Her2^{high} but not Her2^{low} cells was significantly enhanced by the addition of +15GFP protein to C6.5 using 0.03 μM of proteins. The Y-axis represents the level of Her2 expression, and X-axis represents the level of GFP protein internalized in the cells. The median GFP fluorescence level of the two cell populations, AU565 (Her2^{high}) and MDA-MB-468 (Her2^{Low}), were quantified and compared. See Tables 8 (first set) and 9 (second set).

TABLE 8
Median Fluorescence Values for the First Set of Experiments

| | | MDA-MB-468 cells (Her2−) | | | AUS65 cells (Her2+) | | |
|---|---|---|---|---|---|---|---|
| | | GFP alone | GFP-C6.5 | C6.5-GFP | GFP alone | GFP-C6.5 | C6.5-GFP |
| Untreated | | 4,303 | | | 6,875 | | |
| +6GFP | 1 uM | 20,301 | 17,338 | | 16,922 | 42,664 | |
| | 0.3 uM | 10,066 | 10,973 | | 9,991 | 36,036 | |
| +9GFP | 1 uM | 68,702 | 75,934 | | 56,556 | 114,459 | |
| | 0.3 uM | 43,710 | 47,111 | | 33,996 | 78,583 | |
| | 0.1 uM | 27,878 | 28,638 | | 21,927 | 58,627 | |
| +15GFP | 1 uM | 320,358 | 155,734 | 306,260 | 252,446 | 180,065 | 822,351 |
| | 0.3 uM | 65,409 | 76,116 | 82,571 | 48,901 | 128,374 | 305,944 |
| | 0.1 uM | 14,270 | 36,343 | 36,070 | 15,146 | 74,673 | 162,337 |
| | 0.03 uM | 5,844 | 13,012 | 13,355 | 8,171 | 37,663 | 75,821 |

TABLE 9
Median Fluorescence Values for the Second Set of Experiments

| | | | Her2− | | Her2+ | |
|---|---|---|---|---|---|---|
| | | | | GFP- | | GFP- |
| | | | GFP | C6.5 | GFP | C6.5 |
| Untreated | | | 4,776 | | 11,162 | |
| sfGFP | 0.3 | uM | 5,245 | 4,947 | 12,131 | 28,995 |
| | 0.1 | uM | 4,519 | 4,824 | 10,937 | 77,366 |
| | 0.03 | uM | | 4,460 | | 15,064 |
| +6GFPb | 0.3 | uM | 87,210 | 29,300 | 72,094 | 88,610 |
| | 0.1 | uM | 35,278 | 15,444 | 28,033 | 58,642 |
| | 0.03 | uM | | 7,288 | | 35,072 |
| +12GFPa | 0.3 | uM | 27,216 | 24,554 | 23,240 | 64,678 |
| | 0.1 | uM | 12,042 | 12,751 | 12,658 | 46,822 |
| | 0.03 | uM | 7,445 | 6,529 | 10,823 | 24,233 |
| +12GFPc | 0.3 | uM | 324,584 | 213,846 | 219,496 | 291,661 |
| | 0.1 | uM | 167,884 | 148,713 | 116,048 | 222,997 |
| | 0.03 | uM | 19,192 | 45,989 | 20,586 | 92,518 |

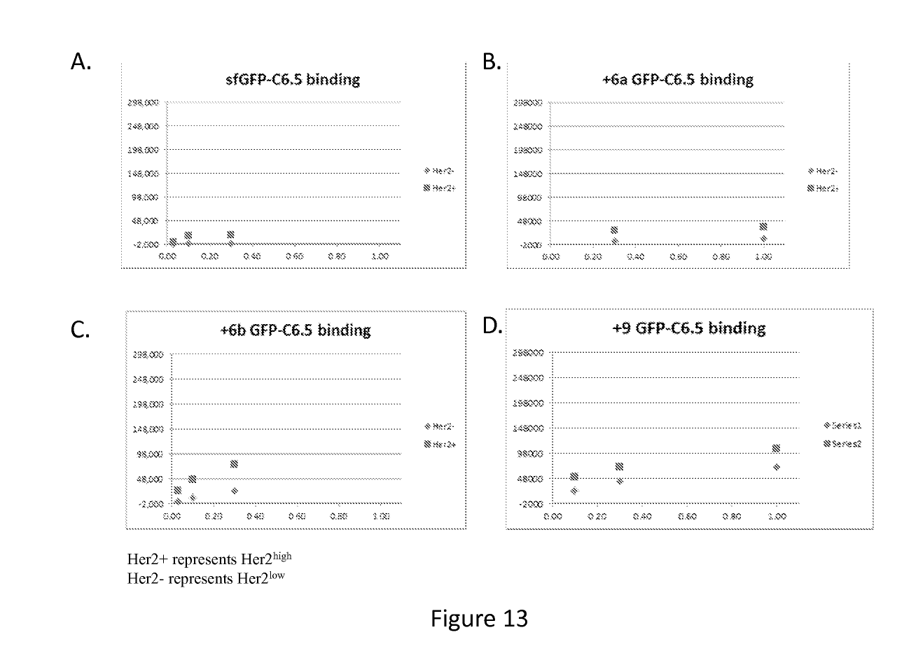

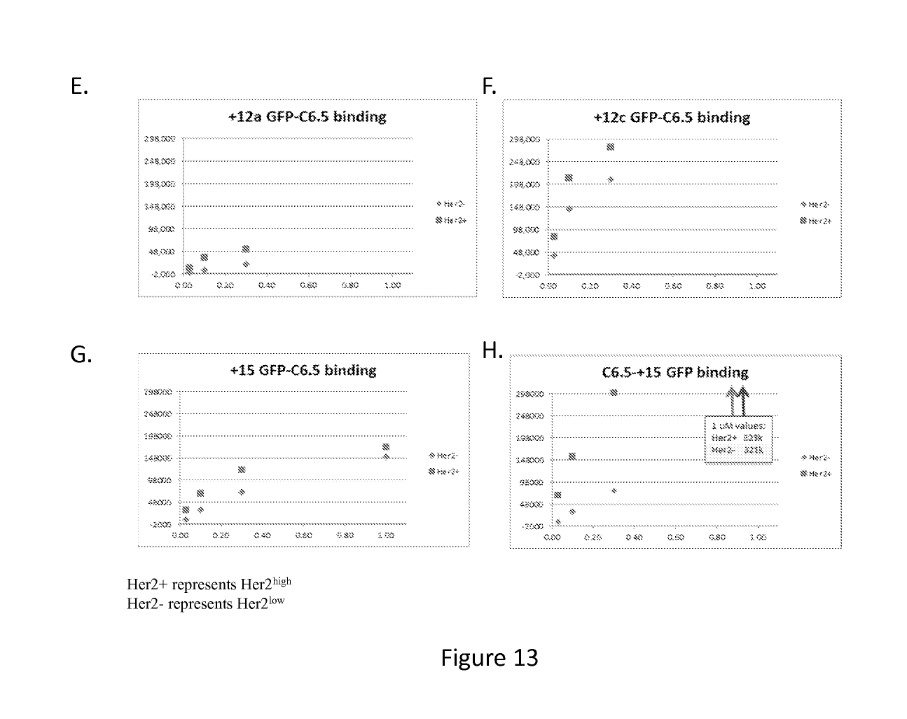

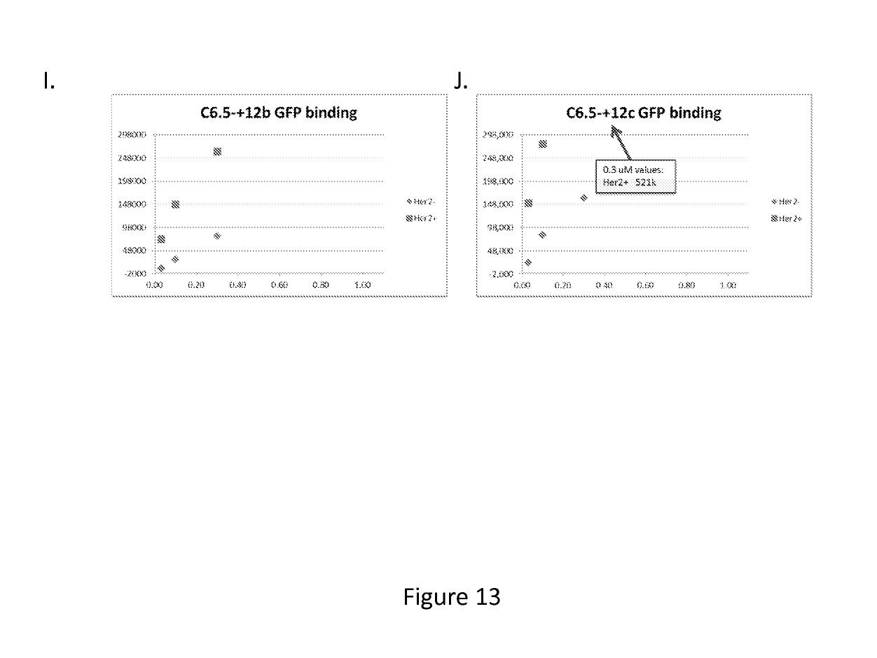

The above data were also plotted in FIGS. 13A-13H to show the median fluorescence value minus background fluorescence of untreated cells (background adjusted fluorescence) (Y-axis) as a function of concentration (X-axis) for each of the tested proteins in this Example. Cellular uptake of the proteins was measured by GFP fluorescence. Her2 expression level was measured by using a Her2 antibody conjugated with allophycocyanin (APC). Gating was applied to the flow cytometry data to identify Her2¹′ versus Her2^{high} populations. The two concentration profiles represent the background adjusted fluorescence for the two cell populations present in the wells, i.e., the Her2^{high} cells (AU565) and the Her2^{Low} cells (MDA-MB-468). The Her2^{low} profiles (diamond) are indicative of the profile of charged GFP alone. The Her2^{high} profiles (square) are indicative of the profile of the charged GFP in combination with the target-binding region—C6.5 scFv. The data of sfGFP-C6.5 on the Her2^{high} cells reflects the profile of the target-binding region (C6.5) by itself.

The above data also show the following:
The binding profile of sfGFP-C6.5 appears to be reflective of the IC50 value of C6.5—indicating no increase in cell internalization using this negatively charged GFP moiety (e.g., a moiety that is not a CPM).
+6b GFP-C6.5 expected binding curve is mostly maintained and substantial difference between Her2^{low} and Her2^{high} cells was observed.
The differences of binding profiles between +6a GFP-C6.5 and +6c GFP-C6.5 and between +12a GFP-C6.5 and +12c GFP-C6.5 indicate that charge distribution also affects the penetration of the fusion proteins.

The results of this Example indicate that charge may be used to enhance internalization of a target-binding region that binds to its target, e.g., a cell-surface receptor, in a concentration-dependent manner. Moreover, internalization is a function of targeting moiety/target interactions, as our results different depending on the level of expression of the target on the cells used. Similarly, internalization will also be a function of the K_{D} of the target-binding region for the target.

The above results also suggest that, to maintain specificity of internalization (e.g., internalization into cells that express the cell surface target recognized by the target-binding region), there is a balance. Too much charge on the CPM region may cause non-specific association with the cell surface and decrease the extent to which protein entity internalization is targeted (e.g., overwhelm the contribution of the target-binding region). The above results also suggest that the binding site accessibility of the target-binding region for its target, e.g., cell-surface receptor, may affect the amount of charge needed.

### Example 6

### Time Course Studies in Mixed Cell Populations Show that Fusion Proteins Comprising a Target-Binding Region and a CPM Retain Cell-Surface Receptor Specific Binding and have Enhanced Internalization

Materials and methods: 100,000 of each of AU565 (Her2^{high}) and MDA-MB-468 (Her2^{Low}) cells were plated in each well of 12-well plate in growth media overnight. The media were replaced with serum free media containing 0.1 μM of protein listed below and incubated for 10 minutes, 30 minutes or 4 hours. Cells were washed 3×PBS, trypsinized, stained with Her2 Antibody-APC for 0.5 hours, washed with PBS and then analyzed by flow cytometry with detection of GFP. The following proteins were tested in this Example:
sfGFP-(S₄G)₆-C6.5-His₆
sfGFP-His₆
+9GFP-(S₄G)₆-C6.5-His₆
+9GFP-His₆
+15GFPc-(S₄G)₆-C6.5-His₆
+15GFPc-His₆

Results are provided in Table 10, which shows the fold increase of cellular uptake in Her2^{high} vs. Her2^{Low} cells for the tested proteins.

TABLE 10

| | | | Her2− | | | Her2+ | | |
|---|---|---|---|---|---|---|---|---|
| | | | GFP | GFP-C6.5 | C6.5-GFP | GFP | GFP-C6.5 | C6.5-GFP |
| Untreated | | | 4134 | | | 6,081 | | |
| sfGFP | 10 | min | 3,887 | 4,081 | | 5,952 | 8,423 | |
| | 30 | min | 4,025 | 3,986 | | 6,024 | 11,836 | |
| | 4 | h | 4,067 | 4,704 | | 5,924 | 43,779 | |
| +9GFP | 10 | min | 7,775 | 5,151 | | 8,762 | 11,943 | |
| | 30 | min | 10,075 | 6,360 | | 9,953 | 18,165 | |
| | 4 | h | 34,081 | 36,830 | | 23,005 | 68,727 | |
| +15GFP | 10 | min | 5,728 | 10,665 | 13,517 | 7,465 | 18,606 | 37,044 |
| | 30 | min | 8,107 | 22,262 | 17,194 | 9,724 | 35,981 | 61,007 |
| | 4 | h | 16,708 | 144,923 | 96,599 | 14,417 | 148,261 | 184,844 |

The results of this Example indicate that charge can be used to enhance internalization of a target-binding region that binds to its target, e.g., a cell-surface receptor. The level of cellular uptake increases over time. Too much charge or too long incubation time may overwhelm the interaction between the target-binding region and its target. The binding affinity of the target-binding region to its target receptor affects the amount of charge needed. Applying charge to the target-binding region may provide additional advantages, such as preferential binding to a specific cell population if time of treatment is limited (such as in vivo).

### Example 7

### A Cytotoxic Agent—Bleomycin is Administered with a Protein Entity Comprising a Target-Binding Region and a CPM for Enhancing Cell Death

Bleomycin is an antineoplastic agent that has been used in the treatment of cancer for several decades. Bleomycin has been shown to have enhanced activity if an endosomal escape agent is used in combination with bleomycin (Bioconjug Chem. 1997 November-December; 8(6):781-4, Listeriolysin O potentiates immunotoxin and bleomycin cytotoxicity).

Materials and Methods: A series of fusion proteins with various charges comprising C6.5 scFv fused to a series of charged GFPs (for example, the charged GFPs produced in Example 1) are administered to cells simultaneously with bleomycin. Bleomycin is administered in trans or is conjugated to the scFv-charged GFP fusion series. Bleomycin is conjugated to the protein using a heterobifunctional linker such as succinimidyl-4-[N-maleimidomethyl]cyclohexane-1-carboxylate (SMCC) wherein a free amine of a bleomycin species is conjugated to the linker via NHS ester group, and an accessible cysteine on the protein is used to conjugate to the maleimide group on the linker. Alternatively, bleomycin is conjugated by dimethyladipimidate treatment (1980) Biochem. J. 185, 787-790. Cell viability of cell lines expressing Her2 receptor and having low Her2 receptor expression are monitored over time at various concentrations of the tested proteins. Cell lines expressing Her2 receptor that can be used in this Example include AU565 breast cancer cells, SKOV-3 ovarian cancer cells, and H2987 human lung adenocarcinoma cells. Cell viability is assessed by MTS assay.

Results: Under the same conditions, administration of bleomycin together with C6.5-charged GFP fusion proteins kill more cells than administration of bleomycin alone.

The results of this Example indicate that a protein entity comprising a target-binding region and a CPM enhances cell death when administered with a cytotoxic agent (either in trans or conjugated). Furthermore, co-administration of a protein entity comprising a target-binding region and a CPM, and a cytotoxic agent (in trans with or conjugated to the protein entity) enhances cell death better than using the cytotoxic agent alone. Such cytotoxic agent is internalized into cells in a receptor-mediated process.

### Example 8

### A Cytotoxic Agent—Maytansinoid DM1 is Administered with a Protein Entity Comprising a Target-Binding Region and a CPM for Enhancing Cell Death

Materials and Methods: A series of fusion proteins with various charges comprising C6.5 scFv fused to a series of charged GFPs (for example, the charged GFPs produced in Example 1) are co-administered simultaneously with Herceptin antibody conjugated to maytansinoid DM1 (known as Trastuzumab emtansine or T-DM1). Cell viability of cell lines expressing Her2 receptor and having low Her2 receptor expression are monitored over time at various concentrations of the tested proteins and compared to that of suitable controls. For example, suitable controls include measuring cell viability following culture with the same fusion proteins in the absence of T-DM1, or following culture with T-DM1 alone. Cell lines expressing Her2 that can be used in this Example include AU565 breast cancer cells, SKOV-3 ovarian cancer cells, and H2987 human lung adenocarcinoma cells. Cell viability is assessed by MTS assay.

Results: Under the same conditions, administration of T-DM1 with C6.5—charged GFP fusion proteins kill more cells than administration of maytansinoid DM1 or its analog alone. Administration of the protein entity alone does not negatively impact cell viability.

### Sequences

(+2)GFPa-His6
MGSASKGEELFTGVVPILVELDGDVNGHKFSVRGEGEGDATNGKLTLKFICTTGKLP
VPWPTLVTTLTYGVQCFSRYPDHMKQHDFFKSAMPEGYVQERTISFKDDGTYKTRA
EVKFEGDTLVNRIELKGIDFKEDGNILGHKLEYNFNSHNVYITADKRKNGIKANFKIR
HNVKDGSVQLADHYQQNTPIGRGPVLLPRNHYLSTRSALSKDPKEKRDHMVLLEFV
TAAGITHGMDELYKGHGHHHHHH
(+2)GFPb-His6
MGSASKGERLFTGVVPILVELDGDVNGHKFSVRGKGKGDATRGKLTLKFICTTGKLP
VPWPTLVTTLTYGVQCFSRYPKHMKQHDFFKSAMPEGYVQERTISFKDDGTYKTRA
EVKFEGDTLVNRIELKGIDFKEDGNILGHKLEYNFNSHNVYITADKQKNGIKANFKIR
HNVEDGSVQLADHYQQNTPIGDGPVLLPDNHYLSTQSALSKDPNEKRDHMVLLEFV
TAAGITHGMDELYKGHGHHHHHH
(+6)GFPa-His6
MGSASKGERLFTGVVPILVELDGDVNGHKFSVRGEGEGDATRGKLTLKFICTTGKLP
VPWPTLVTTLTYGVQCFSRYPKHMKRHDFFKSAMPEGYVQERTISFKKDGTYKTRA
EVKFEGRTLVNRIELKGRDFKEKGNILGHKLEYNFNSHNVYITADKQKNGIKANFKI
RHNVEDGSVQLADHYQQNTPIGDGPVLLPDNHYLSTQSALSKDPNEKRDHMVLLEF
VTAAGITHGMDELYKGHGHHHHHH
(+6)GFPb-His6
MGSASKGERLFRGKVPILVELKGDVNGHKFSVRGKGKGDATRGKLTLKFICTTGKLP
VPWPTLVTTLTYGVQCFSRYPKHMKQHDFFKSAMPEGYVQERTISFKDDGTYKTRA
EVKFEGDTLVNRIELKGIDFKEDGNILGHKLEYNFNSHNVYITADKQKNGIKANFKIR
HNVEDGSVQLADHYQQNTPIGDGPVLLPDNHYLSTQSALSKDPNEKRDHMVLLEFV
TAAGITHGMDELYKGHGHHHHHH
(+9)GFP-His6
MGSASKGEELFTGVVPILVELDGDVNGHKFSVRGEGEGDATNGKLTLKFICTTGKLP
VPWPTLVTTLTYGVQCFSRYPDHMKRHDFFKSAMPKGYVQERTISFKKDGKYKTRA
EVKFEGRTLVNRIKLKGRDFKEKGNILGHKLRYNFNSHKVYITADKQKNGIKANFKI
RHNVEDGSVQLADHYQQNTPIGDGPVLLPDNHYLSTQSALSKDPNEKRDHMVLLEF
VTAAGITHGMDELYKGHGHHHHHH
(+12)GFPa-His6
MGSASKGERLFTGVVPILVELDGDVNGHKFSVRGEGEGDATRGKLTLKFICTTGKLP
VPWPTLVTTLTYGVQCFSRYPKHMKRHDFFKSAMPKGYVQERTISFKKDGTYKTRA
EVKFEGRTLVNRIKLKGRDFKEKGNILGHKLRYNFNSHNVYITADKQKNGIKANFKI
RHNVEDGSVQLADHYQQNTPIGDGPVLLPDNHYLSTQSALSKDPNEKRDHMVLLEF
VTAAGITHGMDELYKGHGHHHHHH
(+12)GFPb-His6
MGSASKGERLFTGVVPILVELDGDVNGHKFSVRGEGEGDATRGKLTLKFICTTGKLP
VPWPTLVTTLTYGVQCFSRYPKHMKQHDFFKSAMPEGYVQERTISFKDDGTYKTRA
EVKFEGDTLVNRIELKGIDFKEDGNILGHKLEYNFNSHNVYITADKRKNGIKAKFKIR
HNVKDGSVQLADHYQQNTPIGRGPVLLPRNHYLSTRSKLSKDPKEKRDHMVLLEFV
TAAGIKHGRDERYKGHGHHHHHH
(+12)GFPc-His6
MGSASKGEELFTGVVPILVELDGDVNGHKFSVRGEGEGDATNGKLTLKFICTTGKLP
VPWPTLVTTLTYGVQCFSRYPDHMKQHDFFKSAMPEGYVQERTISFKDDGTYKTRA
EVKFEGDTLVNRIELKGIDFKEDGNILGHKLEYNFNSHNVYITADKRKNGIKAKFKIR
HNVKDGSVQLAKHYQQNTPIGRGPVLLPRKHYLSTRSKLSKDPKEKRDHMVLLEFV
TAAGIKHGRKERYKGHGHHHHHH
(+15)GFP-His6
MGSASKGERLFTGVVPILVELDGDVNGHKFSVRGEGEGDATRGKLTLKFICTTGKLP
VPWPTLVTTLTYGVQCFSRYPKHMKRHDFFKSAMPEGYVQERTISFKKDGTYKTRA
EVKFEGRTLVNRIELKGRDFKEKGNILGHKLEYNFNSHNVYITADKRKNGIKANFKIR
HNVKDGSVQLADHYQQNTPIGRGPVLLPRNHYLSTRSALSKDPKEKRDHMVLLEFV
TAAGITHGMDELYKGHGHHHHHH
sfGFP-His6
MGSASKGEELFTGVVPILVELDGDVNGHKFSVRGEGEGDATNGKLTLKFICTTGKLP
VPWPTLVTTLTYGVQCFSRYPDHMKQHDFFKSAMPEGYVQERTISFKDDGTYKTRA
EVKFEGDTLVNRIELKGIDFKEDGNILGHKLEYNFNSHNVYITADKQKNGIKANFKIR
HNVEDGSVQLADHYQQNTPIGDGPVLLPDNHYLSTQSALSKDPNEKRDHMVLLEFV
TAAGITHGMDELYKGHGHHHHHH
His6-C6.5
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHG
C6.5-His6
MGSQVQLLQSGAELKKPGESLKISCKGSGYSFTSYWIAWVRQMPGKGLEYMGLIYP
GDSDTKYSPSFQGQVTISVDKSVSTAYLQWSSLKPSDSAVYFCARHDVGYCSSSNCA
KWPEYFQHWGQGTLVTVSSGGGGSGGGGSGGGGSQSVLTQPPSVSAAPGQKVTISC
SGSSSNIGNNYVSWYQQLPGTAPKLLIYGHTNRPAGVPDRFSGSKSGTSASLAISGFR
SEDEADYYCAAWDDSLSGWVFGGGTKLTVLGGHGHHHHHH
sfGFP-(S₄G)₆-C6.5-His6
MGSASKGEELFTGVVPILVELDGDVNGHKFSVRGEGEGDATNGKLTLKFICTTGKLP
VPWPTLVTTLTYGVQCFSRYPDHMKQHDFFKSAMPEGYVQERTISFKDDGTYKTRA
EVKFEGDTLVNRIELKGIDFKEDGNILGHKLEYNFNSHNVYITADKQKNGIKANFKIR
HNVEDGSVQLADHYQQNTPIGDGPVLLPDNHYLSTQSALSKDPNEKRDHMVLLEFV
TAAGITHGMDELYKGHGSSSSGSSSSGSSSSGSSSSGSSSSGSSSSGSQVQLLQSGAEL
KKPGESLKISCKGSGYSFTSYWIAWVRQMPGKGLEYMGLIYPGDSDTKYSPSFQGQV
TISVDKSVSTAYLQWSSLKPSDSAVYFCARHDVGYCSSSNCAKWPEYFQHWGQGTL
VTVSSGGGGSGGGGSGGGGSQSVLTQPPSVSAAPGQKVTISCSGSSSNIGNNYVSWY
QQLPGTAPKLLIYGHTNRPAGVPDRFSGSKSGTSASLAISGFRSEDEADYYCAAWDD
SLSGWVFGGGTKLTVLGGHGHHHHHH
(+15)GFP-(S₄G)₆-C6.5-His6
MGSASKGERLFTGVVPILVELDGDVNGHKFSVRGEGEGDATRGKLTLKFICTTGKLP
VPWPTLVTTLTYGVQCFSRYPKHMKRHDFFKSAMPEGYVQERTISFKKDGTYKTRA
EVKFEGRTLVNRIELKGRDFKEKGNILGHKLEYNFNSHNVYITADKRKNGIKANFKIR
HNVKDGSVQLADHYQQNTPIGRGPVLLPRNHYLSTRSALSKDPKEKRDHMVLLEFV
TAAGITHGMDELYKGHGSSSSGSSSSGSSSSGSSSSGSSSSGSSSSGSQVQLLQSGAEL
KKPGESLKISCKGSGYSFTSYWIAWVRQMPGKGLEYMGLIYPGDSDTKYSPSFQGQV
TISVDKSVSTAYLQWSSLKPSDSAVYFCARHDVGYCSSSNCAKWPEYFQHWGQGTL
VTVSSGGGGSGGGGSGGGGSQSVLTQPPSVSAAPGQKVTISCSGSSSNIGNNYVSWY
QQLPGTAPKLLIYGHTNRPAGVPDRFSGSKSGTSASLAISGFRSEDEADYYCAAWDD
SLSGWVFGGGTKLTVLGGHGHHHHHH
C6.5-(S₄G)₆-sfGFP-His6
MGSQVQLLQSGAELKKPGESLKISCKGSGYSFTSYWIAWVRQMPGKGLEYMGLIYP
GDSDTKYSPSFQGQVTISVDKSVSTAYLQWSSLKPSDSAVYFCARHDVGYCSSSNCA
KWPEYFQHWGQGTLVTVSSGGGGSGGGGSGGGGSQSVLTQPPSVSAAPGQKVTISC
SGSSSNIGNNYVSWYQQLPGTAPKLLIYGHTNRPAGVPDRFSGSKSGTSASLAISGFR
SEDEADYYCAAWDDSLSGWVFGGGTKLTVLGGHGSSSSGSSSSGSSSSGSSSSGSSSS
GSSSSGSASKGEELFTGVVPILVELDGDVNGHKFSVRGEGEGDATNGKLTLKFICTTG
KLPVPWPTLVTTLTYGVQCFSRYPDHMKQHDFFKSAMPEGYVQERTISFKDDGTYK
TRAEVKFEGDTLVNRIELKGIDFKEDGNILGHKLEYNFNSHNVYITADKQKNGIKAN
FKIRHNVEDGSVQLADHYQQNTPIGDGPVLLPDNHYLSTQSALSKDPNEKRDHMVL
LEFVTAAGITHGMDELYKGHGHHHHHH
C6.5-(S₄G)₆-(+15)GFP-His6
MGSQVQLLQSGAELKKPGESLKISCKGSGYSFTSYWIAWVRQMPGKGLEYMGLIYP
GDSDTKYSPSFQGQVTISVDKSVSTAYLQWSSLKPSDSAVYFCARHDVGYCSSSNCA
KWPEYFQHWGQGTLVTVSSGGGGSGGGGSGGGGSQSVLTQPPSVSAAPGQKVTISC
SGSSSNIGNNYVSWYQQLPGTAPKLLIYGHTNRPAGVPDRFSGSKSGTSASLAISGFR
SEDEADYYCAAWDDSLSGWVFGGGTKLTVLGGHGSSSSGSSSSGSSSSGSSSSGSSSS
GSSSSGSASKGERLFTGVVPILVELDGDVNGHKFSVRGEGEGDATRGKLTLKFICTTG
KLPVPWPTLVTTLTYGVQCFSRYPKHMKRHDFFKSAMPEGYVQERTISFKKDGTYK
TRAEVKFEGRTLVNRIELKGRDFKEKGNILGHKLEYNFNSHNVYITADKRKNGIKAN
FKIRHNVKDGSVQLADHYQQNTPIGRGPVLLPRNHYLSTRSALSKDPKEKRDHMVL
LEFVTAAGITHGMDELYKGHGHHHHHH
(+2)GFPa-(S₄G)₆-C6.5_scFv-His6
MGSASKGEELFTGVVPILVELDGDVNGHKFSVRGEGEGDATNGKLTLKFICTTGKLP
VPWPTLVTTLTYGVQCFSRYPDHMKQHDFFKSAMPEGYVQERTISFKDDGTYKTRA
EVKFEGDTLVNRIELKGIDFKEDGNILGHKLEYNFNSHNVYITADKRKNGIKANFKIR
HNVKDGSVQLADHYQQNTPIGRGPVLLPRNHYLSTRSALSKDPKEKRDHMVLLEFV
TAAGITHGMDELYKGHGSSSSGSSSSGSSSSGSSSSGSSSSGSSSSGSQVQLLQSGAEL
KKPGESLKISCKGSGYSFTSYWIAWVRQMPGKGLEYMGLIYPGDSDTKYSPSFQGQV
TISVDKSVSTAYLQWSSLKPSDSAVYFCARHDVGYCSSSNCAKWPEYFQHWGQGTL
VTVSSGGGGSGGGGSGGGGSQSVLTQPPSVSAAPGQKVTISCSGSSSNIGNNYVSWY
QQLPGTAPKLLIYGHTNRPAGVPDRFSGSKSGTSASLAISGFRSEDEADYYCAAWDD
SLSGWVFGGGTKLTVLGGHGHHHHHH
(+2)GFPb-(S₄G)₆-C6.5_scFv-His6
MGSASKGERLFTGVVPILVELDGDVNGHKFSVRGKGKGDATRGKLTLKFICTTGKLP
VPWPTLVTTLTYGVQCFSRYPKHMKQHDFFKSAMPEGYVQERTISFKDDGTYKTRA
EVKFEGDTLVNRIELKGIDFKEDGNILGHKLEYNFNSHNVYITADKQKNGIKANFKIR
HNVEDGSVQLADHYQQNTPIGDGPVLLPDNHYLSTQSALSKDPNEKRDHMVLLEFV
TAAGITHGMDELYKGHGSSSSGSSSSGSSSSGSSSSGSSSSGSSSSGSQVQLLQSGAEL
KKPGESLKISCKGSGYSFTSYWIAWVRQMPGKGLEYMGLIYPGDSDTKYSPSFQGQV
TISVDKSVSTAYLQWSSLKPSDSAVYFCARHDVGYCSSSNCAKWPEYFQHWGQGTL
VTVSSGGGGSGGGGSGGGGSQSVLTQPPSVSAAPGQKVTISCSGSSSNIGNNYVSWY
QQLPGTAPKLLIYGHTNRPAGVPDRFSGSKSGTSASLAISGFRSEDEADYYCAAWDD
SLSGWVFGGGTKLTVLGGHGHHHHHH
(+6)GFPa-(S₄G)₆-C6.5_scFv-His6
MGSASKGERLFTGVVPILVELDGDVNGHKFSVRGEGEGDATRGKLTLKFICTTGKLP
VPWPTLVTTLTYGVQCFSRYPKHMKRHDFFKSAMPEGYVQERTISFKKDGTYKTRA
EVKFEGRTLVNRIELKGRDFKEKGNILGHKLEYNFNSHNVYITADKQKNGIKANFKI
RHNVEDGSVQLADHYQQNTPIGDGPVLLPDNHYLSTQSALSKDPNEKRDHMVLLEF
VTAAGITHGMDELYKGHGSSSSGSSSSGSSSSGSSSSGSSSSGSSSSGSQVQLLQSGAE
LKKPGESLKISCKGSGYSFTSYWIAWVRQMPGKGLEYMGLIYPGDSDTKYSPSFQGQ
VTISVDKSVSTAYLQWSSLKPSDSAVYFCARHDVGYCSSSNCAKWPEYFQHWGQGT
LVTVSSGGGGSGGGGSGGGGSQSVLTQPPSVSAAPGQKVTISCSGSSSNIGNNYVSW
YQQLPGTAPKLLIYGHTNRPAGVPDRFSGSKSGTSASLAISGFRSEDEADYYCAAWD
DSLSGWVFGGGTKLTVLGGHGHHHHHH
(+6)GFPb-(S₄G)₆-C6.5-His6
MGSASKGERLFRGKVPILVELKGDVNGHKFSVRGKGKGDATRGKLTLKFICTTGKLP
VPWPTLVTTLTYGVQCFSRYPKHMKQHDFFKSAMPEGYVQERTISFKDDGTYKTRA
EVKFEGDTLVNRIELKGIDFKEDGNILGHKLEYNFNSHNVYITADKQKNGIKANFKIR
HNVEDGSVQLADHYQQNTPIGDGPVLLPDNHYLSTQSALSKDPNEKRDHMVLLEFV
TAAGITHGMDELYKGHGSSSSGSSSSGSSSSGSSSSGSSSSGSSSSGSQVQLLQSGAEL
KKPGESLKISCKGSGYSFTSYWIAWVRQMPGKGLEYMGLIYPGDSDTKYSPSFQGQV
TISVDKSVSTAYLQWSSLKPSDSAVYFCARHDVGYCSSSNCAKWPEYFQHWGQGTL
VTVSSGGGGSGGGGSGGGGSQSVLTQPPSVSAAPGQKVTISCSGSSSNIGNNYVSWY
QQLPGTAPKLLIYGHTNRPAGVPDRFSGSKSGTSASLAISGFRSEDEADYYCAAWDD
SLSGWVFGGGTKLTVLGGHGHHHHHH
(+9)GFP-(S₄G)₆-C6.5-His6
MGSASKGEELFTGVVPILVELDGDVNGHKFSVRGEGEGDATNGKLTLKFICTTGKLP
VPWPTLVTTLTYGVQCFSRYPDHMKRHDFFKSAMPKGYVQERTISFKKDGKYKTRA
EVKFEGRTLVNRIKLKGRDFKEKGNILGHKLRYNFNSHKVYITADKQKNGIKANFKI
RHNVEDGSVQLADHYQQNTPIGDGPVLLPDNHYLSTQSALSKDPNEKRDHMVLLEF
VTAAGITHGMDELYKGHGSSSSGSSSSGSSSSGSSSSGSSSSGSSSSGSQVQLLQSGAE
LKKPGESLKISCKGSGYSFTSYWIAWVRQMPGKGLEYMGLIYPGDSDTKYSPSFQGQ
VTISVDKSVSTAYLQWSSLKPSDSAVYFCARHDVGYCSSSNCAKWPEYFQHWGQGT
LVTVSSGGGGSGGGGSGGGGSQSVLTQPPSVSAAPGQKVTISCSGSSSNIGNNYVSW
YQQLPGTAPKLLIYGHTNRPAGVPDRFSGSKSGTSASLAISGFRSEDEADYYCAAWD
DSLSGWVFGGGTKLTVLGGHGHHHHHH
(+12)GFPa-(S₄G)₆-C6.5-His6
MGSASKGERLFTGVVPILVELDGDVNGHKFSVRGEGEGDATRGKLTLKFICTTGKLP
VPWPTLVTTLTYGVQCFSRYPKHMKRHDFFKSAMPKGYVQERTISFKKDGTYKTRA
EVKFEGRTLVNRIKLKGRDFKEKGNILGHKLRYNFNSHNVYITADKQKNGIKANFKI
RHNVEDGSVQLADHYQQNTPIGDGPVLLPDNHYLSTQSALSKDPNEKRDHMVLLEF
VTAAGITHGMDELYKGHGSSSSGSSSSGSSSSGSSSSGSSSSGSSSSGSQVQLLQSGAE
LKKPGESLKISCKGSGYSFTSYWIAWVRQMPGKGLEYMGLIYPGDSDTKYSPSFQGQ
VTISVDKSVSTAYLQWSSLKPSDSAVYFCARHDVGYCSSSNCAKWPEYFQHWGQGT
LVTVSSGGGGSGGGGSGGGGSQSVLTQPPSVSAAPGQKVTISCSGSSSNIGNNYVSW
YQQLPGTAPKLLIYGHTNRPAGVPDRFSGSKSGTSASLAISGFRSEDEADYYCAAWD
DSLSGWVFGGGTKLTVLGGHGHHHHHH
(+12)GFPb-(S₄G)₆-C6.5-His6
MGSASKGERLFTGVVPILVELDGDVNGHKFSVRGEGEGDATRGKLTLKFICTTGKLP
VPWPTLVTTLTYGVQCFSRYPKHMKQHDFFKSAMPEGYVQERTISFKDDGTYKTRA
EVKFEGDTLVNRIELKGIDFKEDGNILGHKLEYNFNSHNVYITADKRKNGIKAKFKIR
HNVKDGSVQLADHYQQNTPIGRGPVLLPRNHYLSTRSKLSKDPKEKRDHMVLLEFV
TAAGIKHGRDERYKGHGSSSSGSSSSGSSSSGSSSSGSSSSGSSSSGSQVQLLQSGAEL
KKPGESLKISCKGSGYSFTSYWIAWVRQMPGKGLEYMGLIYPGDSDTKYSPSFQGQV
TISVDKSVSTAYLQWSSLKPSDSAVYFCARHDVGYCSSSNCAKWPEYFQHWGQGTL
VTVSSGGGGSGGGGSGGGGSQSVLTQPPSVSAAPGQKVTISCSGSSSNIGNNYVSWY
QQLPGTAPKLLIYGHTNRPAGVPDRFSGSKSGTSASLAISGFRSEDEADYYCAAWDD
SLSGWVFGGGTKLTVLGGHGHHHHHH
(+12)GFPc-(S₄G)₆-C6.5-His6
MGSASKGEELFTGVVPILVELDGDVNGHKFSVRGEGEGDATNGKLTLKFICTTGKLP
VPWPTLVTTLTYGVQCFSRYPDHMKQHDFFKSAMPEGYVQERTISFKDDGTYKTRA
EVKFEGDTLVNRIELKGIDFKEDGNILGHKLEYNFNSHNVYITADKRKNGIKAKFKIR
HNVKDGSVQLAKHYQQNTPIGRGPVLLPRKHYLSTRSKLSKDPKEKRDHMVLLEFV
TAAGIKHGRKERYKGHGSSSSGSSSSGSSSSGSSSSGSSSSGSSSSGSQVQLLQSGAEL
KKPGESLKISCKGSGYSFTSYWIAWVRQMPGKGLEYMGLIYPGDSDTKYSPSFQGQV
TISVDKSVSTAYLQWSSLKPSDSAVYFCARHDVGYCSSSNCAKWPEYFQHWGQGTL
VTVSSGGGGSGGGGSGGGGSQSVLTQPPSVSAAPGQKVTISCSGSSSNIGNNYVSWY
QQLPGTAPKLLIYGHTNRPAGVPDRFSGSKSGTSASLAISGFRSEDEADYYCAAWDD
SLSGWVFGGGTKLTVLGGHGHHHHHH
His6-C6.5-(S₄G)₆-(+6)GFPa
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKRHDFFKSAMPEGYVQERTISFK
KDGTYKTRAEVKFEGRTLVNRIELKGRDFKEKGNILGHKLEYNFNSHNVYITADKQ
KNGIKANFKIRHNVEDGSVQLADHYQQNTPIGDGPVLLPDNHYLSTQSALSKDPNEK
RDHMVLLEFVTAAGITHGMDELYKGHGDSK
His6-C6.5-(S₄G)₆-(+6)GFPb
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFRGKVPILVELKGDVNGHKFSVRGKGKGDATRGKLTL
KFICTTGKLPVPWPTLVTTLTYGVQCFSRYPKHMKQHDFFKSAMPEGYVQERTISFK
DDGTYKTRAEVKFEGDTLVNRIELKGIDFKEDGNILGHKLEYNFNSHNVYITADKQK
NGIKANFKIRHNVEDGSVQLADHYQQNTPIGDGPVLLPDNHYLSTQSALSKDPNEKR
DHMVLLEFVTAAGITHGMDELYKGHGDSK
His6-C6.5-(S₄G)₆-(+9)GFP
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGEELFTGVVPILVELDGDVNGHKFSVRGEGEGDATNGKLTL
KFICTTGKLPVPWPTLVTTLTYGVQCFSRYPDHMKRHDFFKSAMPKGYVQERTISFK
KDGKYKTRAEVKFEGRTLVNRIKLKGRDFKEKGNILGHKLRYNFNSHKVYITADKQ
KNGIKANFKIRHNVEDGSVQLADHYQQNTPIGDGPVLLPDNHYLSTQSALSKDPNEK
RDHMVLLEFVTAAGITHGMDELYKGHGDSK
His6-C6.5-(S₄G)₆-(+12)GFPa
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKRHDFFKSAMPKGYVQERTISFK
KDGTYKTRAEVKFEGRTLVNRIKLKGRDFKEKGNILGHKLRYNFNSHNVYITADKQ
KNGIKANFKIRHNVEDGSVQLADHYQQNTPIGDGPVLLPDNHYLSTQSALSKDPNEK
RDHMVLLEFVTAAGITHGMDELYKGHGDSK
His6-C6.5-(S₄G)₆-(+12)GFPb
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKQHDFFKSAMPEGYVQERTISFK
DDGTYKTRAEVKFEGDTLVNRIELKGIDFKEDGNILGHKLEYNFNSHNVYITADKRK
NGIKAKFKIRHNVKDGSVQLADHYQQNTPIGRGPVLLPRNHYLSTRSKLSKDPKEKR
DHMVLLEFVTAAGIKHGRDERYKGHGDSK
His6-C6.5-(S₄G)₆-(+12)GFPc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGEELFTGVVPILVELDGDVNGHKFSVRGEGEGDATNGKLTL
KFICTTGKLPVPWPTLVTTLTYGVQCFSRYPDHMKQHDFFKSAMPEGYVQERTISFK
DDGTYKTRAEVKFEGDTLVNRIELKGIDFKEDGNILGHKLEYNFNSHNVYITADKRK
NGIKAKFKIRHNVKDGSVQLAKHYQQNTPIGRGPVLLPRKHYLSTRSKLSKDPKEKR
DHMVLLEFVTAAGIKHGRKERYKGHGDSK
His6-C6.5-(S₄G)₆-(+15)GFP
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKRHDFFKSAMPEGYVQERTISFK
KDGTYKTRAEVKFEGRTLVNRIELKGRDFKEKGNILGHKLEYNFNSHNVYITADKRK
NGIKANFKIRHNVKDGSVQLADHYQQNTPIGRGPVLLPRNHYLSTRSALSKDPKEKR
DHMVLLEFVTAAGITHGMDELYKGHGDSK
His6-C6.5-(S₄G)₆-sfGFP
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGEELFTGVVPILVELDGDVNGHKFSVRGEGEGDATNGKLTL
KFICTTGKLPVPWPTLVTTLTYGVQCFSRYPDHMKQHDFFKSAMPEGYVQERTISFK
DDGTYKTRAEVKFEGDTLVNRIELKGIDFKEDGNILGHKLEYNFNSHNVYITADKQK
NGIKANFKIRHNVEDGSVQLADHYQQNTPIGDGPVLLPDNHYLSTQSALSKDPNEKR
DHMVLLEFVTAAGITHGMDELYKGHGDSK
His6-C6.5-(S₄G)₆-(+6)GFPa-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKRHDFFKSAMPEGYVQERTISFK
KDGTYKTRAEVKFEGRTLVNRIELKGRDFKEKGNILGHKLEYNFNSHNVYITADKQ
KNGIKANFKIRHNVEDGSVQLADHYQQNTPIGDGPVLLPDNHYLSTQSALSKDPNEK
RDHMVLLEFVTAAGITHGMDELYKGHGEQKLISEEDL
His6-C6.5-(S₄G)₆-(+6)GFPb-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFRGKVPILVELKGDVNGHKFSVRGKGKGDATRGKLTL
KFICTTGKLPVPWPTLVTTLTYGVQCFSRYPKHMKQHDFFKSAMPEGYVQERTISFK
DDGTYKTRAEVKFEGDTLVNRIELKGIDFKEDGNILGHKLEYNFNSHNVYITADKQK
NGIKANFKIRHNVEDGSVQLADHYQQNTPIGDGPVLLPDNHYLSTQSALSKDPNEKR
DHMVLLEFVTAAGITHGMDELYKGHGEQKLISEEDL
His6-C6.5-(S₄G)₆-(+9)GFP-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGEELFTGVVPILVELDGDVNGHKFSVRGEGEGDATNGKLTL
KFICTTGKLPVPWPTLVTTLTYGVQCFSRYPDHMKRHDFFKSAMPKGYVQERTISFK
KDGKYKTRAEVKFEGRTLVNRIKLKGRDFKEKGNILGHKLRYNFNSHKVYITADKQ
KNGIKANFKIRHNVEDGSVQLADHYQQNTPIGDGPVLLPDNHYLSTQSALSKDPNEK
RDHMVLLEFVTAAGITHGMDELYKGHGEQKLISEEDL
His6-C6.5-(S₄G)₆-(+12)GFPa-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKRHDFFKSAMPKGYVQERTISFK
KDGTYKTRAEVKFEGRTLVNRIKLKGRDFKEKGNILGHKLRYNFNSHNVYITADKQ
KNGIKANFKIRHNVEDGSVQLADHYQQNTPIGDGPVLLPDNHYLSTQSALSKDPNEK
RDHMVLLEFVTAAGITHGMDELYKGHGEQKLISEEDL
His6-C6.5-(S₄G)₆-(+12)GFPb-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKQHDFFKSAMPEGYVQERTISFK
DDGTYKTRAEVKFEGDTLVNRIELKGIDFKEDGNILGHKLEYNFNSHNVYITADKRK
NGIKAKFKIRHNVKDGSVQLADHYQQNTPIGRGPVLLPRNHYLSTRSKLSKDPKEKR
DHMVLLEFVTAAGIKHGRDERYKGHGEQKLISEEDL
His6-C6.5-(S₄G)₆-(+12)GFPc-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGEELFTGVVPILVELDGDVNGHKFSVRGEGEGDATNGKLTL
KFICTTGKLPVPWPTLVTTLTYGVQCFSRYPDHMKQHDFFKSAMPEGYVQERTISFK
DDGTYKTRAEVKFEGDTLVNRIELKGIDFKEDGNILGHKLEYNFNSHNVYITADKRK
NGIKAKFKIRHNVKDGSVQLAKHYQQNTPIGRGPVLLPRKHYLSTRSKLSKDPKEKR
DHMVLLEFVTAAGIKHGRKERYKGHGEQKLISEEDL
His6-C6.5-(S₄G)₆-(+15)GFP-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGERLFTGVVPILVELDGDVNGHKFSVRGEGEGDATRGKLTL
KFICTTGKLPVPWPTLVTTLTYGVQCFSRYPKHMKRHDFFKSAMPEGYVQERTISFK
KDGTYKTRAEVKFEGRTLVNRIELKGRDFKEKGNILGHKLEYNFNSHNVYITADKRK
NGIKANFKIRHNVKDGSVQLADHYQQNTPIGRGPVLLPRNHYLSTRSALSKDPKEKR
DHMVLLEFVTAAGITHGMDELYKGHGEQKLISEEDL
His6-C6.5-(S₄G)₆-sfGFP-Myc
MHHHHHHGSQVQLLQSGAELKKPGESLKISCKGSGYSFTSYWIAWVRQMPGKGLE
YMGLIYPGDSDTKYSPSFQGQVTISVDKSVSTAYLQWSSLKPSDSAVYFCARHDVGY
CSSSNCAKWPEYFQHWGQGTLVTVSSGGGGSGGGGSGGGGSQSVLTQPPSVSAAPG
QKVTISCSGSSSNIGNNYVSWYQQLPGTAPKLLIYGHTNRPAGVPDRFSGSKSGTSAS
LAISGFRSEDEADYYCAAWDDSLSGWVFGGGTKLTVLGGHGSSSSGSSSSGSSSSGSS
SSGSSSSGSSSSGSASKGEELFTGVVPILVELDGDVNGHKFSVRGEGEGDATNGKLTL
KFICTTGKLPVPWPTLVTTLTYGVQCFSRYPDHMKQHDFFKSAMPEGYVQERTISFK
DDGTYKTRAEVKFEGDTLVNRIELKGIDFKEDGNILGHKLEYNFNSHNVYITADKQK
NGIKANFKIRHNVEDGSVQLADHYQQNTPIGDGPVLLPDNHYLSTQSALSKDPNEKR
DHMVLLEFVTAAGITHGMDELYKGHGEQKLISEEDL
Myc-(+36)GFP-His6
MEQKLISEEDLGSASKGERLFRGKVPILVELKGDVNGHKFSVRGKGKGDATRGKLTL
KFICTTGKLPVPWPTLVTTLTYGVQCFSRYPKHMKRHDFFKSAMPKGYVQERTISFK
KDGKYKTRAEVKFEGRTLVNRIKLKGRDFKEKGNILGHKLRYNFNSHKVYITADKR
KNGIKAKFKIRHNVKDGSVQLADHYQQNTPIGRGPVLLPRNHYLSTRSKLSKDPKEK
RDHMVLLEFVTAAGIKHGRDERYKGHGHHHHHH
(+36)GFP-His6
MGSASKGERLFRGKVPILVELKGDVNGHKFSVRGKGKGDATRGKLTLKFICTTGKLP
VPWPTLVTTLTYGVQCFSRYPKHMKRHDFFKSAMPKGYVQERTISFKKDGKYKTRA
EVKFEGRTLVNRIKLKGRDFKEKGNILGHKLRYNFNSHKVYITADKRKNGIKAKFKI
RHNVKDGSVQLADHYQQNTPIGRGPVLLPRNHYLSTRSKLSKDPKEKRDHMVLLEF
VTAAGIKHGRDERYKGHGHHHHHH

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A protein entity comprising:
a target binding region that binds a cell surface target with a dissociation constant (K_{D}) of greater than 0.01 nM or with an avidity of greater than 0.001 nM, or with a K_{D} of less than 1 μM or with an avidity of less than 1 μM, and
a charged protein moiety (CPM) that enhances penetration into cells;
wherein the CPM a) has tertiary structure and a molecular weight of at least 4 kDa and has surface positive charge and a net theoretical charge of less than +20; or b) has tertiary structure and a molecular weight of at least 4 kDa and has surface positive charge, a net positive charge of at least +5, and a charge per molecular weight ration of less than 0.75;
wherein the cell surface target is distinct from that bound by the CPM;
and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone.

2-4. (canceled)

5. The protein entity of claim 1, wherein a primary spacer region (SR) a) interconnects the target binding region and the CPM; or b) forms a fusion protein with at least one unit of the target binding region and at least one unit of the CPM.

6-7. (canceled)

8. The protein entity of claim 5, wherein the protein entity further comprises a cargo region connected to at least one of the CPM, the primary SR, or the target binding region.

9. The protein entity of claim 8, wherein the cargo region is selected from a peptide, a protein, or a small molecule.

10. (canceled)

11. The protein entity of claim 5, wherein the primary SR comprises all or a portion of an immunoglobulin (Ig) comprising at least one of a C_{H}1 domain, a hinge region, a C_{H}2 domain, and a C_{H}3 domain.

12. The protein entity of claim 5, wherein the primary SR comprises an immunoglobulin (Ig) C_{H}1 domain that is genetically fused to a hinge region.

13. The protein entity of claim 12, wherein the primary SR further comprises a C_{H}2 domain of an immunoglobulin to interconnect a target binding region to a C-terminal C_{H}3 dimerization domain of an immunoglobulin.

14. The protein entity of claim 12, wherein the CPM comprises a C_{H}3 domain of an immunoglobulin (Ig).

15. The protein entity of claim 14, wherein
the C_{H}3 domain is a charge-engineered variant comprising least 3, at least 4, at least 5, at least 6, at least 7, or at least 8 amino acid substitutions to increase surface positive charge, theoretical net charge, and/or charge per molecular weight ratio.

16-21. (canceled)

22. The protein entity of claim 1, wherein the target binding region is a target-specific Fv region, comprising a light chain variable (V_{L}) domain mated with a heavy chain variable (V_{H}) domain, together forming an antibody binding site that binds the cell surface target with suitable specificity and affinity.

23. The protein entity of claim 22, wherein the target binding region is a target-specific single chain Fv (scFv), comprising a light chain variable (V_{L}) domain fused via a linker of at least 12 residues with a heavy chain variable (V_{H}) domain, together forming an antibody binding site with suitable specificity and affinity.

24-26. (canceled)

27. The protein entity of claim 14, wherein the protein entity comprises an immunoglobulin (Ig) C_{H}3 domain which has been altered to increase its surface positive charge and/or net positive charge to enhance penetration into cells.

28-34. (canceled)

35. The protein entity of claim 27, wherein, altering of the amino acid sequence comprises introducing at least 3, at least 4, at least 5, at least 6, at least 7, or at least 8 amino acid substitutions, independently, into one or, if present, both C_{H}3 domains to increase surface positive charge, net positive charge, and/or charge per molecular weight ratio of the CPM.

36. (canceled)

37. (canceled)

38. The protein entity of claim 1, wherein the target binding region comprises an antibody fragment, and wherein the antibody fragment is a single-chain antibody (scFv), an F(ab′)2 fragment, an Fab fragment, or an Fd fragment.

39-48. (canceled)

49. The protein entity of claim 1, wherein the penetration of the protein entity into cells that express the cell surface target is increased relative to that of the target binding region alone.

50. The protein entity of claim 1, wherein the targeting specificity of the protein entity is increased relative to that of the CPM alone.

51-58. (canceled)

59. The protein entity of claim 1, wherein the CPM is a variant having at least two amino acid substitutions, additions, or deletions relative to a starting protein, and wherein the CPM has a greater net theoretical charge than the starting protein by at least +2.

60. (canceled)

61. The protein entity of claim 59, wherein the CPM is a variant having at least three, at least four, at least five, at least six, at least seven, at least 8, at least 9, or at least 10 amino acid substitutions relative to a starting protein.

62. (canceled)

63. The protein entity of claim 59, wherein the CPM has a greater net theoretical charge than the starting protein by at least +3, at least +4, at least +5, at least +6, at least +7, at least +8, at least +9, at least +10, at least +12, at least +14, at least +16, or at least +18.

64. (canceled)

65. The protein entity of claim 5, wherein the primary SR comprises a flexible peptide or polypeptide linker.

66. The protein entity of claim 65, wherein the flexible peptide or polypeptide linker comprises a plurality of glycine and serine residues.

67-76. (canceled)

77. The protein entity of claim 5, wherein the SR comprises:
(S₄G)₂-[Cys-(S₄G]₄-(S₄G)₂

78-84. (canceled)

85. A fusion protein comprising:
a target binding portion that binds a cell surface target with a dissociation constant (K_{D}) of greater than 0.01 nM or with an avidity of greater than 0.001 nM, or with a K_{D} of less than 1 μM or with an avidity of less than 1 μM, and
a CPM that enhances penetration into cells;
wherein the CPM a) is a polypeptide having tertiary structure and a molecular weight of at least 4 kDa and has surface positive charge and a net theoretical charge of less than +20; or b) is a polypeptide having tertiary structure, a molecular weight of at least 4 kDa and a theoretical net charge of at least +5 and has surface positive charge and a charge per molecular weight ratio of less than 0.75;
wherein the cell surface target is distinct from that bound by the CPM;
and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone.

86. (canceled)

87. A fusion protein comprising:
a first polypeptide portion comprising a target binding region that binds a cell surface target with a dissociation constant (K_{D}) of less than 1 μM or with an avidity of less than 1 μM, and
a second polypeptide portion comprising a CPM that enhances penetration into cells;
wherein the CPM a) is a polypeptide having tertiary structure and a molecular weight of at least 4 kDa and has surface positive charge and a net theoretical charge of less than +20; or b) is a polypeptide having tertiary structure and a molecular weight of at least 4 kDa and a theoretical net charge of at least +5, wherein the CPM has surface positive charge and a charge per molecular weight ratio of less than 0.75;
wherein the cell surface target is distinct from that bound by the CPM;
and wherein the protein entity binds the cell surface target with sufficient affinity or avidity to effect penetration of the protein entity into cells that express the cell surface target, wherein penetration of the protein entity into the cells is increased relative to that of at least one of the target binding region alone or the CPM alone.

88-103. (canceled)

104. A nucleic acid comprising a nucleotide sequence encoding the fusion protein of claim 85.

105. A vector comprising the nucleic acid of claim 104.

106. A host cell comprising the vector of claim 105.

107. A method of making a fusion protein, comprising
(i) providing the host cell of claim 106 in culture media and culturing the host cell under suitable condition for expression of protein therefrom; and
(ii) expressing the fusion protein.

108. (canceled)

109. (canceled)

110. A method of delivering a target binding region or a cargo region into cells, comprising
providing the protein entity of claim 1 or the fusion protein of claim 85, wherein said protein entity comprises the target binding region, or wherein said protein entity further comprises a cargo region for delivery into a cell that expresses the cell surface target, and
administering said protein entity or said fusion protein to a subject in need thereof to deliver the protein entity into cells to deliver the target binding region or the cargo region.

111. (canceled)

112. A method of enhancing penetration of a target binding region or of a cargo region into cells, comprising
providing the protein entity of claim 1 or the fusion protein of claim 85, wherein said protein entity comprises the target binding region, or wherein said protein entity further comprises a cargo region for delivery into a cell that expresses the cell surface target, and
contacting cells with said protein entity or said fusion protein or administering said protein entity or said fusion protein to a subject.

113-117. (canceled)

118. A method of enhancing penetration of a co-administered agents into cells, comprising
providing the protein entity of claim 1 or the fusion protein of claim 85,
administering said protein entity or said fusion protein to a subject in need thereof, and
administering said agent to said subject, wherein the agent is administered at the same time, or, within the half-life of the protein entity or the agents, prior to or following administration of the protein entity or fusion protein.

119-129. (canceled)

130. The protein entity of claim 1 or the fusion protein of claim 85, wherein the target binding region is a scFv and the CPM is selected from Table [3].


SEQUENCE LISTING (52 sequences)

SEQ ID NO: 1 - protein, 250 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKGHGHHHHHH

SEQ ID NO: 2 - protein, 250 aa, Artificial Sequence
  Description of Artificial Sequence Synthetic polypeptide
MGSASKGERLFTGVVPILVELDGDVNGHKFSVRGKGKGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGHHHHHH

SEQ ID NO: 3 - protein, 250 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGHHHHHH

SEQ ID NO: 4 - protein, 250 aa, Artificial Sequence
  Description of Artificial Sequence Synthetic polypeptide
MGSASKGERLFRGKVPILVELKGDVNGHKFSVRGKGKGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGHHHHHH

SEQ ID NO: 5 - protein, 250 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKRHDFFKSAMPKGYVQERTISFKKDGKYKTRAEVKFEGRTLVNRIKLKGRDFKEKGNILGHKLRYNFNSHKVYITADKQKNGIKANFKIRHNVEDGSVQLADHYQQNTPIGDGPVLLPDNHYLSTQSALSKDPNEKRDHMVLLEFVTAAGITHGMDELYKGHGHHHHHH

SEQ ID NO: 6 - protein, 250 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKRHDFFKSAMPKGYVQERTISFKKDGTYKTRAEVKFEGRTLVNRIKLKGRDFKEKGNILGHKLRYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGHHHHHH

SEQ ID NO: 7 - protein, 250 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLADHYQQNTPIGRGPVLLPRNHYLSTRSKLSKDPKEKRDHMVLLEFVTAAGIKHGRDERYKGHGHHHHHH

SEQ ID NO: 8 - protein, 250 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLAKHYQQNTPIGRGPVLLPRKHYLSTRSKLSKDPKEKRDHMVLLEFVTAAGIKHGRKERYKGHGHHHHHH

SEQ ID NO: 9 - protein, 250 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKGHGHHHHHH

SEQ ID NO: 10 - protein, 250 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGHHHHHH

SEQ ID NO: 11 - protein, 267 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHG

SEQ ID NO: 12 - protein, 267 aa, Artificial Sequence
  Description of Artificial Sequence Synthetic polypeptide
MGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 13 - protein, 539 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 14 - protein, 539 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 15 - protein, 539 aa, Artificial Sequence
  Description of Artificial Sequence Synthetic polypeptide
MGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGHHHHHH

SEQ ID NO: 16 - protein, 539 aa, Artificial Sequence
  Description of Artificial Sequence Synthetic polypeptide
MGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKGHGHHHHHH

SEQ ID NO: 17 - protein, 539 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 18 - protein, 539 aa, Artificial Sequence
  Description of Artificial Sequence Synthetic polypeptide
MGSASKGERLFTGVVPILVELDGDVNGHKFSVRGKGKGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 19 - protein, 539 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 20 - protein, 539 aa, Artificial Sequence
  Description of Artificial Sequence Synthetic polypeptide
MGSASKGERLFRGKVPILVELKGDVNGHKFSVRGKGKGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 21 - protein, 539 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKRHDFFKSAMPKGYVQERTISFKKDGKYKTRAEVKFEGRTLVNRIKLKGRDFKEKGNILGHKLRYNFNSHKVYITADKQKNGIKANFKIRHNVEDGSVQLADHYQQNTPIGDGPVLLPDNHYLSTQSALSKDPNEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 22 - protein, 539 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKRHDFFKSAMPKGYVQERTISFKKDGTYKTRAEVKFEGRTLVNRIKLKGRDFKEKGNILGHKLRYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 23 - protein, 539 aa, Artificial Sequence
  Description of Artificial Sequence Synthetic polypeptide
MGSASKGERLFTGVVPILVELDGDVNGHKFSVRGEGEGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLADHYQQNTPIGRGPVLLPRNHYLSTRSKLSKDPKEKRDHMVLLEFVTAAGIKHGRDERYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 24 - protein, 539 aa, Artificial Sequence
  Description of Artificial Sequence Synthetic polypeptide
MGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLAKHYQQNTPIGRGPVLLPRKHYLSTRSKLSKDPKEKRDHMVLLEFVTAAGIKHGRKERYKGHGSSSSGSSSSGSSSSGSSSSGSSSSGSSSSGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGHHHHHH

SEQ ID NO: 25 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGDSK

SEQ ID NO: 26 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFRGKVPILVELKGDVNGHKFSVRGKGKGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGDSK

SEQ ID NO: 27 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGEELFTGVVPILVELDGDVNGHKFSVRGEGEGDATNGKLTLKFICTTGKLPVPWPTLVTTLTYGVQCFSRYPDHMKRHDFFKSAMPKGYVQERTISFKKDGKYKTRAEVKFEGRTLVNRIKLKGRDFKEKGNILGHKLRYNFNSHKVYITADKQKNGIKANFKIRHNVEDGSVQLADHYQQNTPIGDGPVLLPDNHYLSTQSALSKDPNEKRDHMVLLEFVTAAGITHGMDELYKGHGDSK

SEQ ID NO: 28 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKRHDFFKSAMPKGYVQERTISFKKDGTYKTRAEVKFEGRTLVNRIKLKGRDFKEKGNILGHKLRYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGDSK

SEQ ID NO: 29 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLADHYQQNTPIGRGPVLLPRNHYLSTRSKLSKDPKEKRDHMVLLEFVTAAGIKHGRDERYKGHGDSK

SEQ ID NO: 30 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLAKHYQQNTPIGRGPVLLPRKHYLSTRSKLSKDPKEKRDHMVLLEFVTAAGIKHGRKERYKGHGDSK

SEQ ID NO: 31 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKGHGDSK

SEQ ID NO: 32 - protein, 542 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGDSK

SEQ ID NO: 33 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGEQKLISEEDL

SEQ ID NO: 34 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFRGKVPILVELKGDVNGHKFSVRGKGKGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGEQKLISEEDL

SEQ ID NO: 35 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGEELFTGVVPILVELDGDVNGHKFSVRGEGEGDATNGKLTLKFICTTGKLPVPWPTLVTTLTYGVQCFSRYPDHMKRHDFFKSAMPKGYVQERTISFKKDGKYKTRAEVKFEGRTLVNRIKLKGRDFKEKGNILGHKLRYNFNSHKVYITADKQKNGIKANFKIRHNVEDGSVQLADHYQQNTPIGDGPVLLPDNHYLSTQSALSKDPNEKRDHMVLLEFVTAAGITHGMDELYKGHGEQKLISEEDL

SEQ ID NO: 36 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKRHDFFKSAMPKGYVQERTISFKKDGTYKTRAEVKFEGRTLVNRIKLKGRDFKEKGNILGHKLRYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGEQKLISEEDL

SEQ ID NO: 37 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLADHYQQNTPIGRGPVLLPRNHYLSTRSKLSKDPKEKRDHMVLLEFVTAAGIKHGRDERYKGHGEQKLISEEDL

SEQ ID NO: 38 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKRKNGIKAKFKIRHNVKDGSVQLAKHYQQNTPIGRGPVLLPRKHYLSTRSKLSKDPKEKRDHMVLLEFVTAAGIKHGRKERYKGHGEQKLISEEDL

SEQ ID NO: 39 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKGHGEQKLISEEDL

SEQ ID NO: 40 - protein, 549 aa, Artificial Sequence
  Description of Artificial Sequence Synthetic polypeptide
MHHHHHHGSQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGHGSSSSGSSSSGSSSSGSSSSGSSSSGSSSSGSASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGHGEQKLISEEDL

SEQ ID NO: 41 - protein, 260 aa, Artificial Sequence
  Description of Artificial Sequence Synthetic polypeptide
MEQKLISEEDLGSASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKGHGHHHHHH

SEQ ID NO: 42 - protein, 250 aa, Artificial Sequence
  Description of Artificial Sequence Synthetic polypeptide
MGSASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKGHGHHHHHH

SEQ ID NO: 43 - protein, 6 aa, Artificial Sequence
  Description of Artificial Sequence Synthetic 6xHis tag
HHHHHH

SEQ ID NO: 44 - protein, 44 aa, Artificial Sequence
  Description of Artificial Sequence Synthetic polypeptide
SSSSGSSSSGCSSSSGCSSSSGCSSSSGCSSSSGSSSSGSSSSG

SEQ ID NO: 45 - protein, 6 aa, Artificial Sequence
  Description of Artificial Sequence Synthetic peptide
XAGVFX

SEQ ID NO: 46 - protein, 6 aa, Artificial Sequence
  Description of Artificial Sequence Synthetic peptide
XGFLGX

SEQ ID NO: 47 - protein, 4 aa, Artificial Sequence
  Description of Artificial Sequence Synthetic peptide
XFKX

SEQ ID NO: 48 - protein, 4 aa, Artificial Sequence
  Description of Artificial Sequence Synthetic peptide
XALX

SEQ ID NO: 49 - protein, 6 aa, Artificial Sequence
  Description of Artificial Sequence Synthetic peptide
XALALX

SEQ ID NO: 50 - protein, 7 aa, Artificial Sequence
  Description of Artificial Sequence Synthetic peptide
XALALAX

SEQ ID NO: 51 - protein, 8 aa, Artificial Sequence
  Description of Artificial Sequence Synthetic peptide
WSHPQFEK

SEQ ID NO: 52 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD